SEQUENCE LISTING (85 sequences)

SEQ ID NO: 1 - protein, 121 aa, synthetic construct
QVHLQQSATELARPGASVKMSCKASGYTFTSYTMHWVKQRPGQGLEWIGYIDPGSGYSEFNHKFKDKSTLTADKSSSTAYMQLISLTSEDSAVYYCARRQDNSGYDSFDYWGQGTTLTVSS

SEQ ID NO: 2 - protein, 121 aa, synthetic construct
QVQLQQPGSELMRPGASVKLSCKASGYTFTSYWMHWVKQRHGQGLEWIGNIYPGSGRTIYDEKFKSKATLTVDTSSSTAYMHLSSLTSEDSAVFYCTRSHYGSSFDAMDYWGQGTSVTVSS

SEQ ID NO: 3 - protein, 121 aa, synthetic construct
KVQLQQSGAGLVKPGASVKLSCKTSGYTFSDYIINWVKQRSGQGLEWIGWFYPGSGSINYNEKFKDKATLTADKSSSTVYMDLSRLTSEDSAVYFCARREDRGGYDAFDYWGQGTTLTVSS

SEQ ID NO: 4 - protein, 118 aa, synthetic construct
EVQLQQSGPELEKPGASVKMSCKASGYSFTDYNMNWVKQSNGKSLEWIGIIDPYYGATTYNQRFKGKATLTVDKSSSTAYMQLKSLTSEDSAVYYCARFGFYGAMDYWGQGISVTVSS

SEQ ID NO: 5 - protein, 121 aa, synthetic construct
KVQLQQSGAGLVKPGASVKLSCKASGYTFSDYIINWVKQRSGQGLEWIGWFYPGSGSINYNEKFKDKATLTADKSSSTVYMDLSRLTSEDSAVYFCARREDRGGYDAFDYWGQGTTLTVSS

SEQ ID NO: 6 - protein, 120 aa, synthetic construct
EVKLVESGGGLVQPGGSLKLSCAASGFTFSSYTMSWVRQTPEKRLEWVAYISNGGGTTYYPDTVKGRFTISRDNAKNTLYLQMSSLKSEDTAVYYCARHRPYDYDVLDYWGQGTTLTVSS

SEQ ID NO: 7 - protein, 107 aa, synthetic construct
DIQMTQSPASLSVSVGETVTITCRTSENIYSNLAWYQQKQGKSPQLLVYAATNLADGVSSRFSGSGSGIQYSLKINSLQSEDFGSYYCQHFWGIPWTFGGGTKLEIK

SEQ ID NO: 8 - protein, 107 aa, synthetic construct
DIQVNQSPSSLSASLGDTITITCHASQNIYVWLNWYQQKPGNIPKLLIYKASNLHTGVPSRFSGSGSGTGFTLTISSLQPEDIATYYCQQGQSYPWTFGGGTKLEIK

SEQ ID NO: 9 - protein, 107 aa, synthetic construct
DIQMTQSPASLSVSVGETVTITCRASENIYSNLAWYQQKQGKSPQLLVYAATKLADGVPSRFSGSGSGTQYSLRINSLQSEDFGSYYCQHFWGTPWTFGGGTKLEIK

SEQ ID NO: 10 - protein, 112 aa, synthetic construct
DVVVTQTPLSLPVSFGDQVSISCRSSQSLANSFGNTYLSWYLHKPGQSPQLLIYGISNRFSGVPDRFSGSGSGTDFTLKISTIKPEDLGVYYCLQGTHQPFTFGSGTTLELK

SEQ ID NO: 11 - protein, 107 aa, synthetic construct
DIQMTQSPASLSVSVGETVTITCRTSENIYSNLAWYQQKQGKSPQLLVYAATKLADGVPSRFSGSGSGTQYSLRINSLQPEDFGSYYCQHFWGTPWTFGGGTKLEIK

SEQ ID NO: 12 - protein, 107 aa, synthetic construct
DIVMTQSQKFMSTSVGDRVSITCKASQIVGTDVAWYQQKPGQSPKILIFSASNRYTGVPDRFTGSGSGTDFTLTISNMQSEDLADYFCHQYSRYPYTFGGGTKLEIK

SEQ ID NO: 13 - protein, 5 aa, synthetic construct
SYTMH

SEQ ID NO: 14 - protein, 5 aa, synthetic construct
SYWMH

SEQ ID NO: 15 - protein, 5 aa, synthetic construct
DYIIN

SEQ ID NO: 16 - protein, 5 aa, synthetic construct
DYNMN

SEQ ID NO: 17 - protein, 5 aa, synthetic construct
SYTMS

SEQ ID NO: 18 - protein, 17 aa, synthetic construct
YIDPGSGYSEFNHKFKD

SEQ ID NO: 19 - protein, 17 aa, synthetic construct
NIYPGSGRTIYDEKFKS

SEQ ID NO: 20 - protein, 17 aa, synthetic construct
WFYPGSGSINYNEKFKD

SEQ ID NO: 21 - protein, 17 aa, synthetic construct
IIDPYYGATTYNQRFKG

SEQ ID NO: 22 - protein, 17 aa, synthetic construct
YISNGGGTTYYPDTVKG

SEQ ID NO: 23 - protein, 12 aa, synthetic construct
RQDNSGYDSFDY

SEQ ID NO: 24 - protein, 12 aa, synthetic construct
SHYGSSFDAMDY

SEQ ID NO: 25 - protein, 12 aa, synthetic construct
REDRGGYDAFDY

SEQ ID NO: 26 - protein, 9 aa, synthetic construct
FGFYGAMDY

SEQ ID NO: 27 - protein, 11 aa, synthetic construct
HRPYDYDVLDY

SEQ ID NO: 28 - protein, 11 aa, synthetic construct
RTSENIYSNLA

SEQ ID NO: 29 - protein, 11 aa, synthetic construct
HASQNIYVWLN

SEQ ID NO: 30 - protein, 11 aa, synthetic construct
RASENIYSNLA

SEQ ID NO: 31 - protein, 16 aa, synthetic construct
RSSQSLANSFGNTYLS

SEQ ID NO: 32 - protein, 11 aa, synthetic construct
KASQIVGTDVA

SEQ ID NO: 33 - protein, 4 aa, synthetic construct
CDRL

SEQ ID NO: 34 - protein, 7 aa, synthetic construct
KASNLHT

SEQ ID NO: 35 - protein, 7 aa, synthetic construct
AATKLAD

SEQ ID NO: 36 - protein, 7 aa, synthetic construct
GISNRFS

SEQ ID NO: 37 - protein, 7 aa, synthetic construct
SASNRYT

SEQ ID NO: 38 - protein, 9 aa, synthetic construct
QHFWGIPWT

SEQ ID NO: 39 - protein, 9 aa, synthetic construct
QQGQSYPWT

SEQ ID NO: 40 - protein, 9 aa, synthetic construct
QHFWGTPWT

SEQ ID NO: 41 - protein, 9 aa, synthetic construct
LQGTHQPFT

SEQ ID NO: 42 - protein, 9 aa, synthetic construct
HQYSRYPYT

SEQ ID NO: 43 - DNA, 363 bp, synthetic construct
  source 1..363: mol_type = other DNA
caggtccacctgcagcagtctgcaactgaactggcaagacctggggcctcagtgaagatgtcctgcaaggcttctggctacacctttactagctacacgatgcactgggtaaaacagaggcctggacagggtctggaatggattggatacattgatcctggcagtggatattctgagttcaatcacaagttcaaggacaagagcacattgactgcagacaaatcctccagcacagcctacatgcaactgatcagcctgacatctgaggactctgcggtctattactgtgcaagaaggcaggacaactcgggctacgactcctttgactactggggccaaggcactactctcacagtctcctca

SEQ ID NO: 44 - DNA, 363 bp, synthetic construct
  source 1..363: mol_type = other DNA
caggtccaactgcagcaacctgggtctgagctgatgaggcctggagcttcagtgaagctgtcctgcaaggcttctggctacacattcaccagctactggatgcactgggtgaagcagaggcatggacaaggccttgagtggattggaaatatttatcctggtagtggtagaactatctacgatgagaagttcaagagcaaggccacactgactgtagacacatcctccagcacagcctacatgcacctcagcagcctgacatctgaggactctgcggtcttttactgtacaagatcccactacggtagtagcttcgatgctatggactactggggtcaaggaacctcagtcaccgtctcctca

SEQ ID NO: 45 - DNA, 363 bp, synthetic construct
  source 1..363: mol_type = other DNA
aaggtccagctgcagcagtctggagctgggctggtgaaacccggggcatcagtgaagctgtcctgcaagacttctggctacaccttcagtgactatattataaattgggtaaagcagaggtctggacagggtcttgagtggattgggtggttttaccctggaagtggtagtataaattacaatgagaaattcaaggacaaggccacattgactgcggacaaatcctccagcacggtctatatggaccttagtagattgacgtctgaagactctgcggtctatttctgtgcaagacgcgaagatcggggaggttacgacgcctttgactactggggccaaggcaccactctcacagtctcctca

SEQ ID NO: 46 - DNA, 354 bp, synthetic construct
  source 1..354: mol_type = other DNA
gaggtccagctgcagcagtctggacctgagctggagaagcctggcgcttcagtgaagatgtcctgcaaggcttctggttactcattcactgactacaacatgaactgggtgaagcagagcaatggaaagagccttgagtggattggaattattgatccttactatggtgctactacctacaaccagaggttcaagggcaaggccacattgactgtagacaaatcctccagcacagcctacatgcagctcaagagcctgacatctgaggactctgcagtctattactgtgcaagatttggtttctacggggctatggactactggggtcaaggaatttcagtcaccgtctcctca

SEQ ID NO: 47 - DNA, 354 bp, synthetic construct; identical to SEQ ID NO: 46
  source 1..354: mol_type = other DNA

SEQ ID NO: 48 - DNA, 363 bp, synthetic construct; identical to SEQ ID NO: 45
  source 1..363: mol_type = other DNA

SEQ ID NO: 49 - DNA, 363 bp, synthetic construct
  source 1..363: mol_type = other DNA
aaggtccagctgcagcagtctggagctgggctggtgaaacccggggcatcagtgaagctgtcctgcaaggcttctggctacaccttcagtgactatattataaattgggtaaagcagaggtctggacagggtcttgagtggattgggtggttttaccctggaagtggtagtataaattacaatgagaaattcaaggacaaggccacattgactgcggacaaatcctccagcacagtctatatggaccttagtagattgacatctgaagactctgcggtctatttctgtgcaagacgcgaagatcggggaggttacgacgcctttgactactggggccaaggcaccactctcacagtctcctca

SEQ ID NO: 50 - DNA, 360 bp, synthetic construct
  source 1..360: mol_type = other DNA
gaagtgaagctggtggagtctgggggaggtttagtgcagcctggagggtccctgaaactctcctgtgcagcctctggattcactttcagtagctataccatgtcttgggttcgccagactccagagaagaggctggagtgggtcgcatacattagtaatggtggtggtactacctactatccagacactgtaaagggccgattcaccatctccagagacaatgccaagaacaccctgtacctgcaaatgagcagtctgaagtctgaggacacggccgtgtattactgtgcaaggcataggccctatgattacgacgtccttgactactggggccaaggcaccactctcacagtctcctca

SEQ ID NO: 51 - DNA, 321 bp, synthetic construct
  source 1..321: mol_type = other DNA
gacatccagatgactcagtctccagcctccctatctgtatctgtgggagaaactgtcaccatcacatgtcgaacaagtgagaatatttacagtaatttagcatggtatcagcagaaacagggaaaatctcctcagctcctggtctatgctgcaacaaacttagcagatggtgtgtcatcaaggttcagtggcagtggatcaggcatacagtattccctcaagatcaacagcctgcagtctgaagattttgggagttattactgtcaacatttttggggtattccgtggacgttcggaggaggcaccaagctggaaatcaaa

SEQ ID NO: 52 - DNA, 321 bp, synthetic construct
  source 1..321: mol_type = other DNA
gacatccaggtgaaccagtctccatccagtctgtctgcatcccttggagacacaattaccatcacttgccatgccagtcagaacatttatgtttggttaaactggtaccagcagaaaccaggaaatattcctaaactattgatctataaggcttccaacttgcacacaggcgtcccatcaaggtttagtggcagtggatctgggacaggtttcacattaaccatcagcagcctgcagcctgaagacattgccacttactactgtcaacagggtcaaagttatccgtggacgttcggtggaggcaccaagctggaaatcaaa

SEQ ID NO: 53 - DNA, 321 bp, synthetic construct
  source 1..321: mol_type = other DNA
gacatccagatgactcagtctccagcctccctatctgtttctgtgggagaaactgtcaccatcacatgtcgagcaagtgagaatatttacagtaatttagcatggtatcagcagaaacagggaaaatctcctcagctcctggtctatgctgcaacaaaattagcagatggtgtgccatcaaggttcagtggcagtggatcaggcacacagtattccctcaggatcaacagcctgcagtctgaagattttgggagttattactgtcaacatttttggggtactccgtggacgttcggtggaggcaccaagctggaaatcaaa

SEQ ID NO: 54 - DNA, 321 bp, synthetic construct; identical to SEQ ID NO: 53
  source 1..321: mol_type = other DNA

SEQ ID NO: 55 - DNA, 336 bp, synthetic construct
  source 1..336: mol_type = other DNA
gatgttgtggtgactcaaactccactctccctgcctgtcagctttggagatcaagtttctatctcttgcaggtctagtcagagtcttgcaaacagttttgggaacacctatttgtcttggtacctgcacaagcctggccagtctccacagctcctcatctatgggatttccaacagattttctggggtgccagacaggttcagtggcagtggttcagggacagatttcacactcaagatcagcacaataaagcctgaggacttgggagtgtattactgcttacaaggtacacatcaaccattcacgttcggctcggggacaacgttggaactaaaa

SEQ ID NO: 56 - DNA, 321 bp, synthetic construct; identical to SEQ ID NO: 53
  source 1..321: mol_type = other DNA

SEQ ID NO: 57 - DNA, 321 bp, synthetic construct
  source 1..321: mol_type = other DNA
gacatccagatgactcagtctccagcctccctatctgtatctgtgggagaaactgtcaccatcacatgtcgaacaagtgagaatatttacagtaatttagcatggtatcagcagaaacagggaaaatctcctcagctcctggtctatgctgcaacaaagttagcagatggtgtgccatcaaggttcagtggcagtggatcaggcacacagtattccctcaggatcaacagcctgcagcctgaagattttgggagttattactgtcaacatttttggggtactccgtggacgttcggtggaggcaccaagctggaaatcaaa

SEQ ID NO: 58 - DNA, 321 bp, synthetic construct
  source 1..321: mol_type = other DNA
gacattgtgatgacccagtctcaaaaattcatgtccacatcagtcggagacagggtcagcatcacctgtaaggccagtcagattgtgggtactgatgtagcctggtatcaacagaaaccaggacagtctcctaaaatattgattttttcggcatccaatcggtacactggagtccctgatcgcttcacaggcagtggatctgggacagatttcactctcaccatcagcaatatgcagtctgaagacctggcagattatttctgccatcaatatagcaggtatccgtacacgttcggaggggggaccaagctggaaataaaa

SEQ ID NO: 59 - DNA, 57 bp, synthetic construct
  source 1..57: mol_type = other DNA
atggaaaggcactggatctttctcttcctgttgtcagtaactgcaggtgtccactcc

SEQ ID NO: 60 - DNA, 57 bp, synthetic construct
  source 1..57: mol_type = other DNA
atgggatggagctgtatcatcctcttcttggtagcaacagctacaggtgtccactcc

SEQ ID NO: 61 - DNA, 57 bp, synthetic construct
  source 1..57: mol_type = other DNA
atggaatggtgctgggtctttctcttcctcctgtcagtaactgcaggtgtccactcc

SEQ ID NO: 62 - DNA, 57 bp, synthetic construct
  source 1..57: mol_type = other DNA
atgggatggacctggatctttattttaatcctgtcagtaactacaggtgtccactct

SEQ ID NO: 63 - DNA, 57 bp, synthetic construct
  source 1..57: mol_type = other DNA
atgaatttcgggctcagcttgattttccttgtccttgttttaaaaggtgtcctgtgt

SEQ ID NO: 64 - DNA, 60 bp, synthetic construct
  source 1..60: mol_type = other DNA
atgagtgtgcccactcaggtcctggggttgctgctgctgtggcttacagatgccagatgt

SEQ ID NO: 65 - DNA, 60 bp, synthetic construct
  source 1..60: mol_type = other DNA
atgagggtccttgctgagctcctggggctgctgctgttctgctttttaggtgtgagatgt

SEQ ID NO: 66 - DNA, 60 bp, synthetic construct
  source 1..60: mol_type = other DNA
atgaatttgcctgttcatctcttggtgcttctgttgttctggattcccgcttccagaggt

SEQ ID NO: 67 - DNA, 60 bp, synthetic construct
  source 1..60: mol_type = other DNA
atggagtctcatactcaggcctttgtattcgcgtttctctggttgtctggtgttgatgga

SEQ ID NO: 68-73 - skipped (6 entries)

SEQ ID NO: 74 - protein, 102 aa, Homo sapiens
NKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPP

SEQ ID NO: 75 - protein, 42 aa, Homo sapiens
KHLCPSPLFPGPSKPFWVLVVVGGVLACYSLLVTVAFIIFWV

SEQ ID NO: 76 - protein, 38 aa, synthetic construct
RSKRSRLLHSDYMNMTPRRPGPTRKHYQPYAPPRDFAA

SEQ ID NO: 77 - protein, 105 aa, synthetic construct
NKILVKQSPMLVAYDNAVNLSWKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 78 - protein, 37 aa, synthetic construct
NKILVKQSPMLVAYDNAVNLSYNEKSNGTIIHVKGEE

SEQ ID NO: 79 - protein, 62 aa, synthetic construct
NKILVKQSPMLVAYDNAVNLSYNEKSNGTIIHVKGKHLCPSPLFPGPSKPYAPPRDFAAYRS

SEQ ID NO: 80 - protein, 202 aa, synthetic construct
NKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 81 - protein, 134 aa, synthetic construct
NKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKP

SEQ ID NO: 82 - protein, 27 aa, synthetic construct
FWVLVVVGGVLACYSLLVTVAFIIFWV

SEQ ID NO: 83 - protein, 41 aa, synthetic construct
RSKRSRLLHSDYMNMTPRRPGPTRKHYQPYAPPRDFAAYRS

SEQ ID NO: 84 - protein, 18 aa, synthetic construct
MLRLLLALNLFPSIQVTG

SEQ ID NO: 85 - protein, 217 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2025/0052767 A1
(43) Pub. Date: Feb. 13, 2025

(54) CD28 BINDING ANTIBODIES AND ANTIGEN BINDING FRAGMENTS THEREOF

(71) Applicant: BioLegend, Inc., San Diego, CA (US)

(72) Inventors: Cheng-Rui LI, San Diego, CA (US); Vanda LOPES, San Diego, CA (US); Takatoku OIDA, Tokyo (JP)

(21) Appl. No.: 18/718,326

(22) PCT Filed: Dec. 12, 2022
(86) PCT No.: PCT/US2022/081335

§ 371 Date: Jun. 10, 2024

Related U.S. Application Data
(60) Provisional application No. 63/425,574, filed on Nov. 15, 2022.
(60) Provisional application No. 63/289,060, filed on Dec. 13, 2021.

(51) Int. Cl. G01N 33/68; C07K 16/28

(52) U.S. Cl. CPC G01N 33/6872; C07K 16/2818; C07K 2317/24; G01N 2333/70521

(57) ABSTRACT

Compositions and methods comprising CD28 antibodies and antigen binding fragments thereof are provided.

### CROSS-REFERENCE

This application is a 35 U.S.C. 371 national phase application of International Patent Cooperation Treaty (PCT) Application No. PCT/US2022/081335, filed on Dec. 12, 2022, entitled CD28 BINDING ANTIBODIES AND ANTIGEN BINDING FRAGMENTS THEREOF, naming Cheng-Rui L I et al. as inventors, which claims the benefit of U.S. Provisional Application No. 63/289,060 filed on Dec. 13, 2021, and U.S. Provisional Application No. 63/425,574 filed on Nov. 15, 2022, each of which is incorporated in its entirety herein by reference.

### FIELD OF THE INVENTION

The present disclosure relates, in some aspects, to antibodies or antigen-binding fragments thereof that bind CD28 or a portion thereof, as well as methods, systems and kits for detection of CD28. In certain aspects, the present disclosure relates to antibodies or antigen binding fragments thereof for use in determining levels of CD28 in a sample containing or suspected of containing CD28. In some aspects, the present disclosure relates to antibodies or antigen-binding fragments thereof for use in diagnosing or treating an individual with or suspected of having a disease or disorder associated with CD28.

### BACKGROUND

The T-cell specific surface glycoprotein CD28, also known as CD28, is a member of the immunoglobulin superfamily which acts as a ligand for CD80 and CD86. The function of CD28 is associated with T cell activation, the induction of cell proliferation, cytokine production and promotion of T-cell survival. However, currently available anti-CD28 antibodies and associated methods are limited in their range of both in vitro and in vivo applications. Thus, there remains a need for reagents, devices and methods of detecting CD28, and diagnosing and/or treating CD28 related diseases or disorders. Provided herein are embodiments that meet such needs.

### BRIEF SUMMARY

Provided herein are antibodies, including antigen-binding fragments thereof, that bind all or a portion thereof of CD28, compositions containing such antibodies or antigen-binding fragments thereof, combinations of such antibodies or antigen-binding fragments thereof and methods of use. In particular embodiments, the antibodies or antigen-binding fragments thereof are used in methods of detecting the presence of CD28 through imaging, including molecular, medical and diagnostic imaging.

Provided herein are antibodies or antigen-binding fragments thereof, including those that specifically bind to a CD28, such as a human CD28, wherein the antibodies or antigen-binding fragments contain particular complementarity determining regions (CDRs), including heavy chain CDRs (i.e., CDRH1, CDRH2, and/or CDRH3) and light chain CDRs (i.e., CDRL1, CDRL2, and/or CDRL3), such as any described herein. In some embodiments, the antibody or antigen-binding fragment thereof includes a heavy chain variable domain and a light chain variable domain, such as any described herein.

In some embodiments, the antibody or antigen binding fragment thereof that binds CD28 or a portion thereof is isolated and includes a) an immunoglobulin heavy chain variable domain comprising (i) a heavy chain complementary determining region 1 (CDRH1) comprising the sequence X₁YX₃X₄X₅ (SEQ ID NO: 68), wherein X₁ is S or D; X₃ is T, W, I or N; X₄ is M or I and X₅ is H, N or S; (ii) a heavy chain complementary determining region 2 (CDRH2) comprising the sequence X₁X₂X₃X₄X₅X₆GX₈X₉X₁₀X₁₁X₁₂X₁₃X₁₄X₁₅KX₁₇ (SEQ ID NO: 69), wherein X₁ is Y, N, W or I; X₂ is I or F; X₃ is D, Y or S; X₄ is P or N, X₅ is G or Y; X₆ is S, Y or G; X₅ is Y, R, S, A or T; X₉ is S, T or I; X₁₀ is E, I, N, T or Y; X₁₁ is F or Y; X₁₂ is N, D or P; X₁₃ is H, E, Q or D; X₁₄ is K, R or T; X₁₅ is F or V; and X₁₇ is D, S or G; and (iii) a heavy chain complementary determining region 3 (CDRH3) comprising the sequence X₁X₂X₃X₄X₅X₆X₇X₈X₉X₁₀DY (SEQ ID NO: 70), wherein X₁ is R, S, F or H; X₂ is Q, H, E, G or R; X₃ is D, Y, For P; X₄ is N, G, R, Y or T; X₅ is S, G or D; X₆ is G, S, or no amino acid; X₇ is Y, F, or no amino acid; X₈ is D or no amino acid; X₉ is S, A or V; and X 10 is F, M or L; and
b) an immunoglobulin light chain variable domain comprising (i) a light chain complementary determining region 1 (CDRL1) comprising the sequence X₁X₂SX₄X₅X₆X₇X₈X₉X₁₀X₁₁X₁₂X₁₃X₁₄X₁₅X₁₆ (SEQ ID NO: 71), wherein X₁ is R, H or K; X₂ is T, A or S; X₄ is E or Q; X₅ is N, S or I; X₆ is I, L or V; X₇ is Y, A or G; X₈ is S, V, N or T; X₉ is N, W, S or D; X₁₀ is L, For V; X₁₁ is A, N or G; X₁₂ is N or no amino acid; X₁₃ is T or no amino acid; X₁₄ is Y or no amino acid; X₁₅ is L or no amino acid; and X₁₆ is S or no amino acid; (ii) a light chain complementary determining region 2 (CDRL2) comprising the sequence X₁X₂X₃X₄X₅X₆X₇ (SEQ ID NO: 72), wherein X₁ is A, K, G or S; X₂ is A or I; X₃ is T or S; X₄ is N or K; X₅ is L or R; X₆ is A, H, For Y; and X₇ is D, T or S; and (iii) a light chain complementary determining region 3 (CDRL3) comprising the sequence X₁X₂X₃X₄X₅X₆PX₈T (SEQ ID NO: 73), wherein X₁ is Q, L or H; X₂ is H or Q; X₃ is F, G or Y; X₄ is W, Q, T or S; X₅ is G, S, H or R; X₆ is I, Y, Tor Q; and X₈ is W, For Y.

In some embodiments, the immunoglobulin heavy chain variable domain contains a CDRH1 with the sequence of amino acids set forth in SEQ ID NO: 13, 14, 15, 16 or 17, or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:13, 14, 15, 16 or 17; a CDRH2 with the sequence of amino acids set forth in SEQ ID NO: 18, 19, 20, 21 or 22, or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 18, 19, 20, 21 or 22; and a CDRH3 with the sequence of amino acids set forth in SEQ ID NO: 23, 24, 25, 26 or 27, or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 23, 24, 25, 26 or 27.

In some embodiments, the immunoglobulin light chain variable domain contains a CDRL1 with the sequence of amino acids set forth in SEQ ID NO: 28, 29, 30, 31 or 32, or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 28, 29, 30, 31 or 32; a CDRL2 with the sequence of amino acids set forth in SEQ ID NO: 33, 34, 35, 36 or 37, or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 33, 34, 35, 36 or 37; and a CDRL3 with the sequence of amino acids set forth in SEQ ID NO: 38, 39, 40, 41 or 42, or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 38, 39, 40, 41 or 42.

In some of any embodiments, the CDRH1 contains the sequence of amino acids set forth in SEQ ID NO: 13, 14, 15, 16 or 17, or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 13, 14, 15, 16 or 17; the CDRH2 contains a sequence of amino acids set forth in SEQ ID NO: 18, 19, 20, 21 or 22, or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 18, 19, 20, 21 or 22; the CDRH3 contains a sequence of amino acids set forth in SEQ ID NO: 23, 24, 25, 26 or 27, or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:23, 24, 25, 26 or 27; the CDRL1 contains a sequence of amino acids set forth in SEQ ID NO: 28, 29, 30, 31 or 32, or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 28, 29, 30, 31 or 32; the CDRL2 contains a sequence of amino acids set forth in SEQ ID NO: 33, 34, 35, 36 or 37, or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 33, 34, 35, 36 or 37; the CDRL3 contains a sequence of amino acids set forth in SEQ ID NO: 38, 39, 40, 41 or 42, or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 38, 39, 40, 41 or 42.

In some embodiments, the immunoglobulin heavy chain variable domain contains the amino acid sequence set forth in any of SEQ ID NOS: 1, 2, 3, 4, 5 or 6, or a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity to any of SEQ ID NOS: 1, 3 or 4.

In some embodiments, the immunoglobulin light chain variable domain contains the amino acid sequence set forth in any of SEQ ID NOS: 7, 8, 9, 10, 11 or 12, or a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity to any of SEQ ID NOS: 7, 8, 9, 10, 11 or 12.

In some of any embodiments, the immunoglobulin heavy chain variable domain contains the amino acid sequence set forth in any of SEQ ID NOS: 1, 2, 3, 4, 5 or 6 or a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity to any of SEQ ID NOS: 1, 2, 3, 4, 5 or 6, and the immunoglobulin light chain variable domain contains the amino acid sequence set forth in any of SEQ ID NOS: 7, 8, 9, 10, 11 or 12, or a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity to any of SEQ ID NOS: 7, 8, 9, 10, 11 or 12.

In some of any of the embodiments herein, the antibody or antigen-binding fragment thereof includes an Fc polypeptide having at least 90% (e.g., 90%, 91%, 92%, 93%, 94%, 95%, 96%, 97%, 98%, 99%, or 100%) identity to a sequence of SEQ ID NO:85.

In some of any of the embodiments herein, the antibody or antigen-binding fragment thereof is isolated. In some of any of the embodiments herein, the antibody is a humanized antibody, a chimeric antibody or a human antibody. In some of any of the embodiments herein, the antibody is a murine antibody.

In some of any of the embodiments herein, the antibody is an antigen-binding fragment thereof. In a further embodiment, the antigen-binding fragment thereof is a Fab, Fab′-SH, Fv, scFv, or (Fab′)2 fragment.

In some of any of the embodiments herein, the antibody is a full length or intact antibody. In some embodiments, the antibody or antigen binding fragment thereof further contains an oligonucleotide. In some embodiments, the oligonucleotide contains a sample barcode sequence. In some embodiments, the oligonucleotide further contains a binding site for a primer and an anchor.

In some of any of the embodiments herein, the antibody further comprises a heavy chain constant domain and/or a light chain constant domain. In a further embodiment, the heavy chain and/or light constant domain is murine or human. In some further embodiments, the heavy chain constant domain is IgG1, IgG2a, IgG2b or IgM.

In some of any of the embodiments herein, the antibody is attached to a label. In some embodiments, the antibody or antigen binding fragment thereof is conjugated to a detectable marker or label. In some embodiments, detectable marker or label is conjugated directly to the antigen or antigen binding fragment thereof. In some embodiments, the detectable marker or label is conjugated to the oligonucleotide. In a further embodiment, the label is a fluorescent dye, a fluorescent protein, a radioisotope, a chromophores, a metal ion, gold particles, silver particles, magnetic particles, a polypeptides, an enzyme, streptavidin, biotin, a luminescent compound, or an oligonucleotide.

In another aspect, the disclosure provides an isolated antibody that specifically binds to, wherein the isolated antibody competes binding to the with an antibody described herein.

In some embodiments of the antibodies described herein, the antibody is a monoclonal antibody. In certain embodiments, the antibody is a humanized antibody. In certain embodiments, the antibody comprises one or more human framework regions.

In some aspects, provided herein is a combination of antibodies or antigen-binding fragments thereof, wherein the combination comprises two or more anti-CD28 antibodies or antigen-binding fragments described herein. In some embodiments, the two or more antibodies or antigen-binding fragments comprise one or more first antibody or antigen-binding fragment thereof that binds to a first epitope or region within CD28; and one or more second antibody or antigen-binding fragment thereof that binds to a second epitope or region within CD28. In some further embodiments, the one or more first antibody or antigen-binding fragments thereof, and the one or more second antibody or antigen-binding fragments thereof bind to a non-overlapping epitope or region of CD28 (e.g., human CD28) and/or do not compete for binding to CD28. Further, in some embodiments, the antibody is conjugated to a detectable marker or label. In some embodiments, the at least one of the antibodies or antigen-binding fragments of the combination of two or more anti-CD28 antibodies or antigen-binding fragments described herein, optionally the one or more first antibody or antigen-binding fragment thereof or the one or more second antibody or antigen-binding fragment thereof, is conjugated to a label. In some embodiments, the at least one of the antibodies or antigen-binding fragments, optionally the one or more first antibody or antigen-binding fragment thereof or the one or more second antibody or antigen-binding fragment thereof, is attached or immobilized to a solid support. In some embodiments, the one or more first or second antibody or antigen-binding fragment is attached or immobilized to a solid support and the other of the one or more first or second antibody or antigen-binding fragment is conjugated to a label. In some embodiments, the label is a fluorescent dye, a fluorescent protein, a radioisotope, a chromophore, a metal ion, gold particles, silver particles, magnetic particles, a polypeptide, an enzyme, streptavidin, biotin, a luminescent compound, or an oligonucleotide. In some embodiments, the solid support is a bead, a column, an array, an assay plate, a microwell, a stick, a filter, or a strip. In certain embodiments, the antibody is non-diffusively immobilized on a solid support. In a further embodiment, the device is a rapid detection device or a rapid diagnostic device.

In some embodiments, any of the antibodies or antigen binding fragments thereof bind to all or a portion of CD28 on a sample. In some embodiments, the binding of any of the antibodies or antigen binding fragments thereof to the CD28 allows for detection of CD28 in a sample. In some embodiments, the detection can be on the surface of a cell or intracellular. In some embodiments, the binding induces activation of the cell in a sample (e.g. a T cell). In some embodiments, the activation includes activation of a T cell by a first signal induced via a TCR and a second signal induced by binding of the CD28 to B7-1 (also known as CD80) or B7-2 (also known as CD86). In some embodiments, the activation includes activation of a T cell that does not comprise a first signal induced via a TCR. In some embodiments, the binding enhances production of cytokines or initiates differentiation of the cell into a memory phenotype.

In another embodiments, the present disclosure provides a diagnostic antibody or antigen binding fragment thereof which contains any of the features described herein. In another aspect, the disclosure features an isolated nucleic acid encoding an isolated antibody described herein. In another aspect, the disclosure features an isolated nucleic acid wherein the immunoglobulin heavy chain variable domain contains the sequence of nucleotides set forth in any of SEQ ID NOs: 43-50. In some embodiments, provided herein is an isolated nucleic acid wherein the immunoglobulin light chain variable domain contains the sequence of nucleotides set forth in any of SEQ ID NOs: 51-58. The disclosure also provides an expression vector comprising the nucleic acid described herein. Further, the disclosure also provides an isolated host cell comprising the expression vector described herein.

In another aspect, the disclosure provides a pharmaceutical composition comprising an isolated antibody described herein and a pharmaceutically acceptable carrier.

In another aspect, the disclosure also provides a diagnostic reagent comprising an isolated antibody described herein.

In another aspect, the disclosure also provides a kit comprising an isolated antibody described herein or the diagnostic reagent described herein.

In yet another aspect, the disclosure provides a method of detecting CD28, comprising contacting a sample known or suspected to contain with an antibody described herein. In some embodiments, provided herein are methods of treating or preventing a disease or disorder. In another embodiment, provided herein are methods of diagnosing a disease or disorder. In some embodiments, the provided disclosure can be used in a method of associating the presence or abundance of CD28 with a location of interest of a tissue sample. In some of any embodiments, the antibody or antigen binding fragment thereof can be used to generate a nucleic acid molecule containing all or a portion of the sequence of an attached oligonucleotide. In some embodiments, the provided disclosure can be used to construct a protein library.

In some of any embodiments of the methods provided herein, the antibody or antigen binding portion thereof can be detected by sequencing. In some of any embodiments of the methods provided herein, the antibody or antigen binding portion thereof can be detected by immunofluorescence. In some of any embodiments of the methods provided herein, the antibody or antigen binding portion thereof can be detected by flow cytometry. Certain embodiments are described further in the following description, examples, claims, and drawings.

### BRIEF DESCRIPTION OF THE DRAWINGS

The drawings illustrate certain embodiments of the technology and are not limiting. For clarity and ease of illustration, the drawings are not made to scale and, in some instances, various aspects may be shown exaggerated or enlarged to facilitate an understanding of particular embodiments.

FIGS. 1A-1B show the amino acid sequences of the variable domains of the immunoglobulin heavy (SEQ ID NOS: 1-6) and light (SEQ ID NOS: 7-12) chains of the eight different anti-CD28 antibodies (AB1-8) provided herein. The CDR regions of each of the heavy and light chains are shown in bold and underline. In each of the alignments, three characters (“*”, “:”, and “.”) are used: “*” indicates positions that have a single, fully conserved residue; “:” indicates that one of the following “strong” residue groups is fully conserved: STA; NEQK; NHQK; NDEQ; QHRK; MILV; MILF; HY; and FYW; and “.” indicates that one of the following “weaker” residue groups is fully conserved: CSA; ATV; SAG; STNK; STPA; SGND; SNDEQK; NDEQHK; NEQHRK; FVLIM; and HFY. These are all the positively scoring residue groups that occur in the Gonnet Pam250 matrix. The “strong” and “weak” residue groups are defined as “strong” score>0.5 and “weak” score≤0.5, respectively.

FIGS. 2A-2I depict surface staining of fresh human peripheral blood cells with exemplary anti-CD28 antibodies AB1-AB8 or commercially available antibody control (CA control), analyzed by flow cytometry.

FIGS. 3A-3I depict histogram overlays for fixed (top) and fresh (bottom) human peripheral blood cells labeled with control or exemplary AB1-AB8 anti-CD28 antibodies, analyzed by flow cytometry.

FIGS. 4A-4J depict rates of cell division for purified and isolated T cells incubated with exemplary AB1-AB8 anti-CD28 antibodies in the presence of anti-CD3 (UCHT1) co-stimulation, assessed by flow cytometry.

FIGS. 5A and 5B depict growth and viability of isolated T cells incubated with exemplary AB1-AB8 anti-CD28 antibodies in the presence of anti-CD3 (OKT3) co-stimulation, assessed using ViCell Blu system.

FIGS. 6A-6J depict rates of cell division for purified and isolated T cells incubated with exemplary AB1-AB8 anti-CD28 antibodies in the absence of anti-CD3 co-stimulation, assessed by flow cytometry.

FIGS. 7A-7J depict histogram overlays for Ba/F3 cells were transfected with human CD28 pre-incubated with exemplary anti-CD28 antibodies AB1-AB8 (top) or without pre-incubation with anti-CD28 antibodies (bottom), and biotinylated recombinant CD86+SAV APC, assessed by flow cytometry.

FIGS. 8A-8C depict the ability of exemplary anti-CD28 antibody AB2 to induce T cell activation markers IL-2 (FIG. 8A) and 110 (FIG. 8B), or to support FOXP3+CD4+ T cell growth (FIG. 8C) in the absence of anti-CD3 co-stimulation.

### DETAILED DESCRIPTION

Provided herein are antibodies or antigen binding fragments thereof that bind CD28, nucleic acids encoding such antibodies and antigen-binding fragments thereof, and cells, such as recombinant cells for expressing and production of these antibodies and antigen-binding fragments that can bind to under physiological and/or in vitro conditions. Also provided are methods of producing and using the antibodies and antigen-binding fragments such as in methods for detecting CD28 in a sample from an individual, including methods for laboratory/research purposes (e.g., flow cytometry, ELISA, and/or Western blot), and/or for the use and treatment and/or prevention of various diseases or disorders through the delivery of pharmaceutical or other compositions that contain such antibodies or antigen-binding fragments thereof.

All references cited herein, including patent applications, patent publications, and scientific literature and databases, are herein incorporated by reference in their entirety for all purposes to the same extent as if each individual reference were specifically and individually indicated to be incorporated by reference.

For clarity of disclosure, and not by way of limitation, the detailed description is divided into the subsections that follow. The section headings used herein are for organizational purposes only and are not to be construed as limiting the subject matter described.

### I. Definitions

Unless defined otherwise, all terms of art, notations and other technical and scientific terminology used herein are intended to have the same meaning as is commonly understood by one of ordinary skill in the art to which the subject matter pertains. In some cases, terms with commonly understood meanings are defined herein for clarity and/or for ready reference, and the inclusion of such definitions herein should not necessarily be construed to represent a substantial difference over what is generally understood in the art. It is to be understood that the disclosure provided herein is not limited to particular compositions or biological systems. It is also understood that the terminology used herein is for the purpose of describing particular embodiments only, and is not to be construed as limiting.

The term “antibody” as used herein includes antigen binding fragments thereof that retain binding specificity. For example, there are a number of well characterized antigen binding fragments. Thus, for example, pepsin digests an antibody C-terminal to the disulfide linkages in the hinge region to produce F(ab)′2, a dimer of Fab which itself is a light chain joined to V_{H}-C_{H}I by a disulfide bond. The F(ab)′2 may be reduced under mild conditions to break the disulfide linkage in the hinge region thereby converting the (Fab′)2 dimer into an Fab′ monomer. The Fab′ monomer is essentially an Fab with part of the hinge region (see, Fundamental Immunology, W. E. Paul, ed., Raven Press, N.Y. (1993), for a more detailed description of other antigen binding fragments). While various antigen binding fragments are defined in terms of the digestion of an intact antibody, one of skill will appreciate that fragments can be synthesized de novo either chemically or by utilizing recombinant DNA methodology. Thus, the term antibody, as used herein also includes antigen binding fragments either produced by the modification of whole antibodies or synthesized using recombinant DNA methodologies.

An antibody as described herein can consist of one or more polypeptides substantially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. In some embodiments, the antibody is IgG (e.g., IgG1, IgG2, IgG3, IgG4), IgM, IgA, IgD, or IgE.

A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

In an antibody, substitution variants have at least one amino acid residue removed and a different residue inserted in its place. The sites of greatest interest for substitutional mutagenesis include the hypervariable regions, but framework alterations are also contemplated. Examples of conservative substitutions are described above.

Substantial modifications in the biological properties of the antibody are accomplished by selecting substitutions that differ significantly in their effect on maintaining (a) the structure of the polypeptide backbone in the area of the substitution, for example, as a β-sheet or helical conformation, (b) the charge or hydrophobicity of the molecule at the target site, or (c) the bulk of the side chain. Naturally occurring residues are divided into groups based on common side-chain properties:
(1) Non-polar: Norleucine, Met, Ala, Val, Leu, Ile;
(2) Polar without charge: Cys, Ser, Thr, Asn, Gln;
(3) Acidic (negatively charged): Asp, Glu;
(4) Basic (positively charged): Lys, Arg;
(5) Residues that influence chain orientation: Gly, Pro; and
(6) Aromatic: Trp, Tyr, Phe, His.
   Non-conservative substitutions are made by exchanging a member of one of these classes for another class.

One type of substitution that can be made is to change one or more cysteines in the antibody, which may be chemically reactive, to another residue, such as, without limitation, alanine or serine. For example, there can be a substitution of a non-canonical cysteine. The substitution can be made in a CDR or framework region of a variable domain or in the constant region of an antibody. In some embodiments, the cysteine is canonical (e.g., involved in disulfide bond formation). Any cysteine residue not involved in maintaining the proper conformation of the antibody also may be substituted, generally with serine, to improve the oxidative stability of the molecule and prevent aberrant cross-linking. Conversely, cysteine bond(s) may be added to the antibody to improve its stability, particularly where the antibody is an antigen binding fragment such as an Fv fragment.

Antibodies include V_{H}-V_{L} dimers, including single chain antibodies (antibodies that exist as a single polypeptide chain), such as single chain Fv antibodies (sFv or scFv) in which a variable heavy and a variable light domains are joined together (directly or through a peptide linker) to form a continuous polypeptide. The single chain Fv antibody is a covalently linked V_{H}-V_{L} which may be expressed from a nucleic acid including V_{H}- and V_{L}-encoding sequences either joined directly or joined by a peptide-encoding linker (e.g., Huston, et al. Proc. Nat. Acad. Sci. USA, 85:5879-5883, 1988). While the V_{H} and V_{L} are connected to each as a single polypeptide chain, the V_{H} and V_{L} domains associate non-covalently. Alternatively, the antibody can be another fragment. Other fragments can also be generated, e.g., using recombinant techniques, as soluble proteins or as fragments obtained from display methods. Antibodies can also include diantibodies and miniantibodies. Antibodies of the disclosure also include heavy chain dimers, such as antibodies from camelids. In some embodiments an antibody is dimeric. In other embodiments, the antibody may be in a monomeric form that has an active isotype. In some embodiments the antibody is in a multivalent form, e.g., a trivalent or tetravalent form.

An “antibody fragment” or “antigen binging fragment thereof” comprises a portion of an intact antibody, the antigen binding and/or the variable region of the intact antibody. Antibody fragments or antigen binding fragments thereof, include but are not limited to Fab fragments, Fab′ fragments, F(ab′)₂ fragments, Fv fragments, disulfide-linked Fvs (dsFv), Fd fragments, Fd′ fragments; diabodies; linear antibodies (see U.S. Pat. No. 5,641,870, Example 2; Zapata et al, Protein Eng. 8 (10): 1057-1062 [1995]); single-chain antibody molecules, including single-chain Fvs (scFv) or single-chain Fabs (scFab); antigen-binding fragments of any of the above and multispecific antibodies from antibody fragments.

“Fv” is composed of one heavy- and one light-chain variable region domain linked by non-covalent association. From the folding of these two domains emanate six complementarity determining regions (CDR) (3 in each from the heavy and light chain) that contribute the amino acid residues for antigen binding and confer antigen binding specificity to the antibody. However, even a single variable domain (or half of an Fv comprising only three CDRs specific for an antigen) has the ability to recognize and bind antigen, although, in some cases, at a lower affinity than the entire binding site.

“dsFv” refers to an Fv with an engineered intermolecular disulfide bond, which stabilizes the V_{H}-V_{L} pair.

An “Fd fragment” is a fragment of an antibody containing a variable domain (V_{H}) and one constant region domain (C_{H}I) of an antibody heavy chain.

A “Fab fragment” is an antibody fragment that results from digestion of a full-length immunoglobulin with papain, or a fragment having the same structure that is produced synthetically, e.g., by recombinant methods. A Fab fragment contains a light chain (containing a V_{L} and C_{L}) and another chain containing a variable domain of a heavy chain (V_{H}) and one constant region domain of the heavy chain (C_{H}I).

A “F(ab′)2 fragment” is an antibody fragment that results from digestion of an immunoglobulin with pepsin at pH 4.0-4.5, or a fragment having the same structure that is produced synthetically, e.g., by recombinant methods. The F(ab′)₂ fragment essentially contains two Fab fragments where each heavy chain portion contains an additional few amino acids, including cysteine residues that form disulfide linkages joining the two fragments.

A “Fab′ fragment” is a fragment containing one half (one heavy chain and one light chain) of the F(ab′)₂ fragment.

An “Fd′ fragment” is a fragment of an antibody containing one heavy chain portion of a F(ab′)₂ fragment.

An “Fv′ fragment” is a fragment containing only the V_{H} and V_{L} domains of an antibody molecule.

An “scFv fragment” refers to an antibody fragment that contains a variable light chain (V_{L}) and variable heavy chain (V_{H}), covalently connected by a polypeptide linker in any order. The linker is of a length such that the two variable domains are bridged without substantial interference. Exemplary linkers are (Gly-Ser)ₙ residues with some Glu or Lys residues dispersed throughout to increase solubility.

“Diabodies” are dimeric scFv; diabodies typically have shorter peptide linkers than scFvs, and preferentially dimerize.

As used herein, the terms “variable region” and “variable domain” refer to the portions of the light and heavy chains of an antibody that include amino acid sequences of complementary determining regions (CDRs, e.g., CDRH1, CDRH2, HCR3, CDRL1, CDRL2, and CDRL3) and framework regions (FRs). The variable domain for the heavy and light chains is commonly designated V_{H} and V_{L}, respectively. The variable domain is included on Fab, F(ab′)₂, Fv and scFv antigen binding fragments described herein, and is involved in specific antigen recognition.

As used herein, “complementarity-determining region (CDR)” refers to the three hypervariable regions in each chain that interrupt the four framework regions established by the light and heavy chain variable regions. The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR 1 from the variable domain of the light chain of the antibody in which it is found.

The sequences of the framework regions of different light or heavy chains are relatively conserved within a species. The framework region of an antibody, that is the combined framework regions of the constituent light and heavy chains, serves to position and align the CDRs in three-dimensional space.

The amino acid sequences of the CDRs and framework regions can be determined using various well-known definitions in the art, e.g., Kabat, North method (see, e.g., North et al., J Mol Biol. 406 (2): 228-256, 2011), Chothia, international ImMunoGeneTics database (IMGT), and AbM (see, e.g., Johnson et al., supra; Chothia & Lesk, 1987, Canonical structures for the hypervariable regions of immunoglobulins. J. Mol. Biol. 196, 901-917; Chothia C. et al., 1989, Conformations of immunoglobulin hypervariable regions. Nature 342, 877-883; Chothia C. et al., 1992, structural repertoire of the human V_{H} segments J. Mol. Biol. 227, 799-817; Al-Lazikani et al., J. Mol. Biol 1997, 273 (4)). Definitions of antigen combining sites are also described in the following: Ruiz et al., IMGT, the international ImMunoGeneTics database. Nucleic Acids Res., 28, 219-221 (2000); and Lefranc, M. -P. IMGT, the international ImMunoGeneTics database. Nucleic Acids Res. January 1; 29 (1): 207-9 (2001); MacCallum et al, Antibody-antigen interactions: Contact analysis and binding site topography, J. Mol. Biol., 262 (5), 732-745 (1996); and Martin et al, Proc. Natl Acad. Sci. USA, 86, 9268-9272 (1989); Martin, et al, Methods Enzymol., 203, 121-153, (1991); Pedersen et al, Immunomethods, 1, 126, (1992); and Rees et al, In Sternberg M. J. E. (ed.), Protein Structure Prediction. Oxford University Press, Oxford, 141-172 1996).

As used herein, “chimeric antibody” refers to an immunoglobulin molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region, or portion thereof, having a different or altered antigen specificity; or with corresponding sequences from another species or from another antibody class or subclass.

As used herein, “humanized antibody” refers to an immunoglobulin molecule in CDRs from a donor antibody are grafted onto human framework sequences. Humanized antibodies may also comprise residues of donor origin in the framework sequences. The humanized antibody can also comprise at least a portion of a human immunoglobulin constant region. Humanized antibodies may also comprise residues which are found neither in the recipient antibody nor in the imported CDR or framework sequences. Humanization can be performed using methods known in the art (e.g., Jones et al., Nature 321:522-525; 1986; Riechmann et al., Nature 332:323-327, 1988; Verhoeyen et al., Science 239:1534-1536, 1988); Presta, Curr. Op. Struct. Biol. 2:593-596, 1992; U.S. Pat. No. 4,816,567), including techniques such as “superhumanizing” antibodies (Tan et al., J. Immunol. 169:1119, 2002) and “resurfacing” (e.g., Staelens et al., Mol. Immunol. 43:1243, 2006; and Roguska et al., Proc. Natl. Acad. Sci USA 91:969, 1994).

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The terms “antigen,” “immunogen,” “antibody target,” “target analyte,” and like terms are used herein to refer to a molecule, compound, or complex that is recognized by an antibody, i.e., can be specifically bound by the antibody. The term can refer to any molecule that can be specifically recognized by an antibody, e.g., a polypeptide, polynucleotide, carbohydrate, lipid, chemical moiety, or combinations thereof (e.g., phosphorylated or glycosylated polypeptides, etc.). One of skill will understand that the term does not indicate that the molecule is immunogenic in every context, but simply indicates that it can be targeted by an antibody.

Antibodies bind to an “epitope” on an antigen. The epitope is the localized site on the antigen that is recognized and bound by the antibody. Epitopes can include a few amino acids or portions of a few amino acids, e.g., 5 or 6, or more, e.g., 20 or more amino acids, or portions of those amino acids. In some cases, the epitope includes non-protein components, e.g., from a carbohydrate, nucleic acid, or lipid. In some cases, the epitope is a three-dimensional moiety. Thus, for example, where the target is a protein, the epitope can be comprised of consecutive amino acids, or amino acids from different parts of the protein that are brought into proximity by protein folding (e.g., a discontinuous epitope). The same is true for other types of target molecules that form three-dimensional structures. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed (1996).

A “label” or a “detectable moiety” is a diagnostic agent or component detectable by spectroscopic, radiological, photochemical, biochemical, immunochemical, chemical, or other physical means. Exemplary labels include radiolabels (e.g., ¹¹¹In, ^{99m}Tc, ¹³¹I, ⁶⁷Ga) and other FDA-approved imaging agents. Additional labels include ³²P, fluorescent dyes, electron-dense reagents, enzymes, biotin, digoxigenin, or haptens and proteins or other entities which can be made detectable, e.g., by incorporating a radiolabel into the targeting agent. Any method known in the art for conjugating a nucleic acid or nanocarrier to the label may be employed, e.g., using methods described in Hermanson, Bioconjugate Techniques 1996, Academic Press, Inc., San Diego.

A “labeled” or “tagged” antibody or agent is one that is bound, either covalently, through a linker or a chemical bond, or noncovalently, through ionic, van der Waals, electrostatic, or hydrogen bonds to a label such that the presence of the antibody or agent may be detected by detecting the presence of the label bound to the antibody or agent.

Techniques for conjugating detectable and therapeutic agents to antibodies are well known (see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery” in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review” in Monoclonal Antibodies '84: Biological And Clinical Applications, Pinchera et al. (eds.), pp. 475-506 (1985); and Thorpe et al., “The Preparation And Cytotoxic Properties Of Antibody-Toxin Conjugates”, Immunol. Rev., 62:119-58 (1982)).

The terms “specific for,” “specifically binds,” and like terms refer to a molecule (e.g., antibody or antigen binding fragment) that binds to a target with at least 2-fold greater affinity than non-target compounds, e.g., at least any of 4-fold, 5-fold, 6-fold, 7-fold, 8-fold, 9-fold, 10-fold, 20-fold, 25-fold, 50-fold, or 100-fold greater affinity. For example, an antibody that specifically binds a target (e.g., CD28) will typically bind the target with at least a 2-fold greater affinity than a non-target. Specificity can be determined using standard methods, e.g., solid-phase ELISA immunoassays (see, e.g., Harlow & Lane, Using Antibodies, A Laboratory Manual (1998) for a description of immunoassay formats and conditions that can be used to determine specific immunoreactivity).

The term “binds” with respect to an antibody target (e.g., antigen, analyte, immune complex), typically indicates that an antibody binds a majority of the antibody targets in a pure population (assuming appropriate molar ratios). For example, an antibody that binds a given antibody target typically binds to at least ⅔ of the antibody targets in a solution (e.g., at least any of 75, 80, 85, 90, 91, 92, 93, 94, 95, 96, 97, 98, 99, or 100%). One of skill will recognize that some variability will arise depending on the method and/or threshold of determining binding.

A “control” sample or value refers to a sample that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). A control can also represent an average value or a range gathered from a number of tests or results. One of skill in the art will recognize that controls can be designed for assessment of any number of parameters. For example, a control can be devised to compare therapeutic benefit based on pharmacological data (e.g., half-life) or therapeutic measures (e.g., comparison of benefit and/or side effects). Controls can be designed for in vitro applications. One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST 2.0 sequence comparison algorithms with default parameters described below, or by manual alignment and visual inspection (see, e.g., NCBI web site ncbi.nlm.nih.gov/BLAST/or the like). Such sequences are then said to be “substantially identical.” As described below, the preferred algorithms can account for gaps and the like. Preferably, identity exists over a region that is at least about 25 amino acids or nucleotides in length, or more preferably over a region that is 50-100 or more amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art.

An algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 are used, with the parameters described herein, to determine percent sequence identity for the nucleic acids and proteins of the disclosure. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

The term “nucleic acid” refers to deoxyribonucleotides or ribonucleotides and polymers thereof in either single- or double-stranded form, and complements thereof. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)).

The terms “polypeptide,” “peptide” and “protein” are used interchangeably herein to refer to a polymer of amino acid residues. The terms encompass to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The term “compete”, as used herein with regard to an antibody, means that a first antibody, or an antigen-binding portion thereof, competes for binding with a second antibody, or an antigen-binding portion thereof, where binding of the first antibody with its cognate epitope is detectably decreased in the presence of the second antibody compared to the binding of the first antibody in the absence of the second antibody. The alternative, where the binding of the second antibody to its epitope is also detectably decreased in the presence of the first antibody, can, but need not be the case. That is, a first antibody can inhibit the binding of a second antibody to its epitope without that second antibody inhibiting the binding of the first antibody to its respective epitope. However, where each antibody detectably inhibits the binding of the other antibody with its cognate epitope or ligand, whether to the same, greater, or lesser extent, the antibodies are said to “cross-compete” with each other for binding of their respective epitope(s). Both competing and cross-competing antibodies are encompassed by the present disclosure. Regardless of the mechanism by which such competition or cross-competition occurs (e.g., steric hindrance, conformational change, or binding to a common epitope, or portion thereof, and the like), the skilled artisan would appreciate, based upon the teachings provided herein, that such competing and/or cross-competing antibodies are encompassed and can be useful for the methods disclosed herein.

Numerous types of competitive binding assays are known, for example: solid phase direct or indirect radioimmunoassay (RIA), solid phase direct or indirect enzyme immunoassay (EIA), sandwich competition assay (see Stahli et al., Methods in Enzymology 9:242-253 (1983)); solid phase direct biotin-avidin EIA (see Kirkland et al., J. Immunol. 137:3614-3619 (1986)); solid phase direct labeled assay, solid phase direct labeled sandwich assay (see Harlow and Lane, Antibodies, A Laboratory Manual, Cold Spring Harbor Press (1988)); solid phase direct label RIA using I-125 label (see Morel et al., Molec. Immunol. 25 (1): 7-15 (1988)); solid phase direct biotin-avidin EIA (Cheung et al., Virology 176:546-552 (1990)); and direct labeled RIA (Moldenhauer et al., Scand. J. Immunol. 32:77-82 (1990)). Typically, such an assay involves the use of purified antigen bound to a solid surface or cells bearing either of these, an un-labelled test immunoglobulin and a labeled reference immunoglobulin. Competitive inhibition is measured by determining the amount of label bound to the solid surface or cells in the presence of the test immunoglobulin. Usually the test immunoglobulin is present in excess. Antibodies identified by competition assay (competing antibodies) include antibodies binding to the same epitope as the reference antibody and antibodies binding to an adjacent epitope sufficiently proximal to the epitope bound by the reference antibody for steric hindrance to occur. Usually, when a competing antibody is present in excess, it will inhibit specific binding of a reference antibody to a common antigen by at least 50 or 75%.

The term “CD28” as used herein refers to human CD28 proteins, isoforms or variants thereof, including naturally occurring variants of human CD28, such as splice variants or allelic variants. The amino acid sequence of an exemplary human CD28 is shown in SEQ ID NO: 74. The amino acid sequence of another exemplary human CD28 is shown in SEQ ID NO: 75. The amino acid sequence of yet another exemplary human CD28 is shown in SEQ ID NO: 76. The amino acid sequence of an exemplary human CD28 is shown in SEQ ID NO: 77. The amino acid sequence of another exemplary human CD28 is shown in SEQ ID NO: 78. The amino acid sequence of yet another exemplary human CD28 is shown in SEQ ID NO: 79. The amino acid sequence of yet another exemplary human CD28 is shown in SEQ ID NO: 80. In some embodiments, human CD28 can refer to a variant, such as an allelic variant or splice variant, that exhibits at least or at least about 85%, 86%, 87%, 88%, 89%, 90%, 91%, 92%, 93%, 94%, 95%, 96%, 97%, 98%, or 99% sequence identity to any of SEQ ID NOs: 74-80. In some embodiments, it is understood that the provided antibodies or antigen-binding fragments may exhibit cross-reactive binding to another mammalian CD28 protein, such as murine CD28, or a primate CD28.

Human CD28 isoform 1 amino acid sequence
(SEQ ID NO: 74; UniProt P10747-1)
NKILVKQSPMLVAYDNAVNLSCKYSYNLFSREFRASLHKGLD
SAVEVCVVYGNYSQQLQVYSKTGFNCDGKLGNESVTFYLQNLYVNQTDI
YFCKIEVMYPPPYLDNEKSNGTIIHVKGKHLCPSPLFPGPSKPFWVLVV
VGGVLACYSLLVTVAFIIFWVRSKRSRLLHSDYMNMTPRRPGPTRKHYQ
PYAPPRDFAAYRS
Human CD28 isoform 2 amino acid sequence
(SEQ ID NO: 75; UniProt P10747-2)
KHLCPSPLFPGPSKPFWVLVVVGGVLACYSLLVTVAFIIFWVRSKRSRL
LHSDYMNMTPRRPGPTRKHYQPYAPPRDFAA
Human CD28 isoform 3 amino acid sequence
(SEQ ID NO: 76; UniProt P10747-3)
NKILVKQSPMLVAYDNAVNLSYNEKSNGTIIHVKGKHLCPSP
LFPGPSKPFWVLVVVGGVLACYSLLVTVAFIIFWVRSKRSRLLHSDYMN
MTPRRPGPTRKHYQPYAPPRDFAAYRS
Human CD28 isoform 4 amino acid sequence
(SEQ ID NO: 77; UniProt P10747-4)
NKILVKQSPMLVAYDNAVNLSWKHLCPSPLFPGPSKPFWVLVVVGGVLA
CYSLLVTVAFIIFWVRSKRSRLLHSDYMNMTPRRPGPTRKHYQPYAPPR
DFAAYRS
Human CD28 isoform 5 amino acid sequence
(SEQ ID NO: 78; UniProt P10747-5)
NKILVKQSPMLVAYDNAVNLSYNEKSNGTIIHVKGEE
Human CD28 isoform 6 amino acid sequence
(SEQ ID NO: 79; UniProt P10747-6)
NKILVKQSPMLVAYDNAVNLSYNEKSNGTIIHVKGKHLCPSPLFPGPSK
PYAPPRDFAAYRS
Human CD28 isoform 7 amino acid sequence
(SEQ ID NO: 80; UniProt P10747-7)
NKILVKQSPMLVAYDNAVNLSCKYSYNLFSREFRASLHKGLDSAVEVCV
VYGNYSQQLQVYSKTGFNCDGKLGNESVTFYLQNLYVNQTDIYFCKIEV
MYPPPYLDNEKSNGTIIHVKGKHLCPSPLFPGPSKPFWVLVVVGGVLAC
YSLLVTVAFIIFWVRSKRSRLLHSDYMNMTPRRPGPTRKHYQPYAPPRD
FAAYRS

In some embodiments, any of the exemplary human CD28 amino acid sequences further comprises an extracellular domain. In some embodiments, the extracellular domain comprises the sequence set forth in the SEQ ID NO: 81. In some embodiments, any of the exemplary human CD28 amino acid sequences further comprises a transmembrane domain. In some embodiments, the transmembrane domain comprises the sequence set forth in the SEQ ID NO: 82. In some embodiments, any of the exemplary human CD28 amino acid sequences further comprises a cytoplasmic domain. In some embodiments, the cytoplasmic domain comprises the sequence set forth in the SEQ ID NO: 83. In some embodiments, any of the exemplary human CD28 amino acid sequences further comprises a signal sequence. In some embodiments, the signal sequence comprises the sequence MLRLLLALNLFPSIQVTG, set forth in the SEQ ID NO: 84. In some embodiments, all or a portion thereof of CD28 refers to the extracellular domain of CD28. In some embodiments, all or a portion thereof of CD28 refers to the transmembrane domain of CD28. In some embodiments, all or a portion thereof of CD28 refers to the cytoplasmic domain of CD28.

By “solid support” is meant a non-aqueous matrix to which an antibody according to the provided disclosure can adhere or attach. For example, solid supports include, but are not limited to, a microtiter plate, a membrane (e.g., nitrocellulose), a bead, a dipstick, a thin-layer chromatographic plate, or other solid medium.

As used herein, an “individual” or a “subject” is a mammal. A “mammal” for purposes of treatment includes humans, domestic and farm animals, and zoo, sports, or pet animals, such as dogs, horses, rabbits, cattle, pigs, hamsters, gerbils, mice, ferrets, rats, cats, etc. In some embodiments, the individual or subject is human.

### II. Antibodies that Bind CD28

Provided herein are antibodies, including antigen binding fragments thereof, that specifically bind to CD28. In some embodiments, the provided antibodies include monoclonal antibodies and antigen-binding fragments thereof that bind CD28 that provide superior target specificity, signal-to-noise ratios, and the like as compared to other reported antibodies. Also provided herein are methods for producing anti-CD28 antibodies, and methods for detecting and using such antibodies.

CD28 is the cluster of differentiation nomenclature for the T cell specific surface glycoprotein. CD28 is a member of the immunoglobulin superfamily (IgSF) and is expressed on the surface of cells as a 90 kDa disulfide linked homodimer (Yun et al. Encyclopedia of Immunology; 1998). Although seven isoforms exist, isoform 1 is considered the canonical sequence. CD28 is involved in the T cell activation process, which involves two types of signals, a first signal induced via the T cell receptor (TCR) upon binding to antigenic peptide major histocompatibility complex (pMHC) and a second signal induced via costimulatory receptors, the prototype of which is CD28. CD28 binds to B7-1 (also known as CD80) or B7-2 (also known as CD86). Co-stimulation through CD28 is critically required for naive T cells to achieve clonal expansion, cytokine production and for protection against apoptosis and anergy (Xia et al. Front. Immunol. 2018). In addition to its costimulatory role, CD28 ligation can enhance the production of various cytokines, such as IL-2, IL-4 and IL-10 in conjunction with TCR/CD3 ligation and CD40L co-stimulation, and plays a role in prolonging an activated T cell's fate and initiating its differentiation into the memory phenotype (Chen. Et al. Nature. 2018. 15:734-736).

Although the classical T cell activation process involves the two types of co-stimulatory signals described above, certain monoclonal antibodies have been identified and termed “superagonists” for their ability to activate T regulatory (TREG) cells in the absence of co-stimulation of the TCR by an antigen-presenting cell. (Brown et al. Diseases. 2018).

In some embodiments, any of the antibodies of antigen binding fragments thereof provided herein bind all or a portion of CD28. In some embodiments, any of the antibodies or antigen binding fragments thereof is a CD28 antibody or antigen binding fragment thereof. In some embodiments, the antibody or antigen binding fragment thereof is isolated (e.g., separated from a component of its natural environment (e.g., an animal, a biological sample)). In some embodiments, the anti-antibody is a humanized antibody, or an antigen binding fragment thereof. In some embodiments, the antibody is a derivative of a humanized antibody that binds. In some embodiments, the antibody binds under laboratory conditions (e.g., binds in vitro, binds in a flow cytometry assay, binds in an ELISA). In some embodiments, the antibody binds under physiological conditions (e.g., binds in a cell in a subject).

Generally, the antibodies provided herein comprise at least one immunoglobulin heavy chain variable domain and at least one immunoglobulin light chain variable domain. In some embodiments, an antibody described herein comprises two immunoglobulin heavy chain variable domains and two immunoglobulin light chain variable domains. Typically, each immunoglobulin heavy chain variable domain of the antibody comprises first, second, and third heavy chain complementarity determining regions (CDRs; CDRH1, CDRH2, and CDRH3), and each immunoglobulin light chain variable domain of the antibody comprises first, second, and third light chain CDRs (CDRL1, CDRL2, and CDRL3).

In some embodiments, the antibodies are antigen binding fragments such as Fab, F(ab′)₂, Fv or scFv. The antigen binding fragments can be generated using any means known in the art including, chemical digestion (e.g., papain or pepsin) and recombinant methods. Methods for isolating and preparing recombinant nucleic acids are known to those skilled in the art (see, Sambrook et al., Molecular Cloning. A Laboratory Manual (2d ed. 1989); Ausubel et al., Current Protocols in Molecular Biology (1995)). The antibodies can be expressed in a variety of host cells, including E. coli, other bacterial hosts, yeast, and various higher eukaryotic cells such as the COS, CHO, and HeLa cells lines and myeloma cell lines.

The disclosure provides an isolated antibody or antigen binding fragment thereof that specifically binds to CD28, wherein the isolated antibody comprises: a) an immunoglobulin heavy chain variable domain comprising: (i) a heavy chain complementary determining region 1 (CDRH1) comprising the sequence X₁YX₃X₄X₅ (SEQ ID NO: 68), wherein X₁ is S or D; X₃ is T, W, I or N; X₄ is M or I and X₅ is H, N or S; (ii) a heavy chain complementary determining region 2 (CDRH2) comprising the sequence X₁X₂X₃X₄X₅X₆GX₈X₉X₁₀X₁₁X₁₂X₁₃X₁₄X₁₅KX₁₇ (SEQ ID NO: 69), wherein X₁ is Y, N, W or I; X₂ is I or F; X₃ is D, Y or S; X₄ is P or N, X₅ is G or Y; X₆ is S, Y or G; X₈ is Y, R, S, A or T; X₉ is S, T or I; X₁₀ is E, I, N, T or Y; X₁₁ is F or Y; X₁₂ is N, D or P; X₁₃ is H, E, Q or D; X₁₄ is K, R or T; X₁₅ is F or V; and X₁₇ is D, S or G; and (iii) a heavy chain complementary determining region 3 (CDRH3) comprising the sequence X₁X₂X₃X₄X₅X₆X₇X₈X₉X₁₀DY (SEQ ID NO: 70), wherein X₁ is R, S, F or H; X₂ is Q, H, E, G or R; X₃ is D, Y, F or P; X₄ is N, G, R, Y or T; X₅ is S, G or D; X₆ is G, S, or no amino acid; X₇ is Y, F, or no amino acid; X₈ is D or no amino acid; X₉ is S, A or V; and X₁₀ is F, M or L; and b) an immunoglobulin light chain variable domain comprising: (i) a light chain complementary determining region 1 (CDRL1) comprising the sequence X₁X₂SX₄X₅X₆X₇X₈X₉X₁₀X₁₁X₁₂X₁₃X₁₄X₁₅X₁₆ (SEQ ID NO: 71), wherein X₁ is R, H or K; X₂ is T, A or S; X₄ is E or Q; X₅ is N, S or I; X₆ is I, L or V; X₇ is Y, A or G; X₈ is S, V, N or T; X₉ is N, W, S or D; X₁₀ is L, For V; X₁₁ is A, N or G; X₁₂ is N or no amino acid; X₁₃ is T or no amino acid; X₁₄ is Y or no amino acid; X₁₅ is L or no amino acid; and X₁₆ is S or no amino acid; (ii) a light chain complementary determining region 2 (CDRL2) comprising the sequence X₁X₂X₃X₄X₅X₆X₇ (SEQ ID NO: 72), wherein X₁ is A, K, G or S; X₂ is A or I; X₃ is T or S; X₄ is Nor K; X₅ is L or R; X₆ is A, H, F or Y; and X₇ is D, T or S; and (iii) a light chain complementary determining region 3 (CDRL3) comprising the sequence X₁X₂X₃X₄X₅X₆PX₈T (SEQ ID NO: 73), wherein X₁ is Q, L or H; X₂ is H or Q; X₃ is F, G or Y; X₄ is W, Q, T or S; X₅ is G, S, H or R; X₆ is I, Y, T or Q; and X₈ is W, For Y.

In some embodiments, antibodies of the disclosure can comprise sequences of a heavy chain complementary determining region 1 (CDRH1), an CDRH2, an CDRH3, a light chain complementary determining region 1 (CDRL1), a CDRL2, a CDRL3. Exemplary CDR amino acid sequences and associated SEQ ID NOs are set forth in Table 1, and exemplary amino acid sequences for heavy chain variable domains (V_{H}), and light chain variable domains (V_{L}) and associated SEQ ID NOs are set forth in Table 2.

TABLE 1
Amino acid sequences of CDRs from exemplary anti-CD28
antibodies shown according to Kabat numbering.

| Exemplary | CDR-H1 | CDR-H2 | CDR-H3 |
|---|---|---|---|
| Antibody | (SEQ ID NO) | (SEQ ID NO) | (SEQ ID NO) |
| AB1 | SEQ ID NO: 13 | SEQ ID NO: 18 | SEQ ID NO: 23 |
| AB2 | SEQ ID NO: 14 | SEQ ID NO: 19 | SEQ ID NO: 24 |
| AB3 | SEQ ID NO: 15 | SEQ ID NO: 20 | SEQ ID NO: 25 |
| AB4 | SEQ ID NO: 16 | SEQ ID NO: 21 | SEQ ID NO: 26 |
| AB5 | SEQ ID NO: 16 | SEQ ID NO: 21 | SEQ ID NO: 26 |
| AB6 | SEQ ID NO: 15 | SEQ ID NO: 20 | SEQ ID NO: 25 |
| AB7 | SEQ ID NO: 15 | SEQ ID NO: 20 | SEQ ID NO: 25 |
| AB8 | SEQ ID NO: 17 | SEQ ID NO: 22 | SEQ ID NO: 27 |
| Antibody | CDR-L1 | CDR-L2 | CDR-L3 |
| Name | (SEQ ID NO) | (SEQ ID NO) | (SEQ ID NO) |
| AB1 | SEQ ID NO: 28 | SEQ ID NO: 33 | SEQ ID NO: 38 |
| AB2 | SEQ ID NO: 29 | SEQ ID NO: 34 | SEQ ID NO: 39 |
| AB3 | SEQ ID NO: 30 | SEQ ID NO: 35 | SEQ ID NO: 40 |
| AB4 | SEQ ID NO: 30 | SEQ ID NO: 35 | SEQ ID NO: 40 |
| AB5 | SEQ ID NO: 31 | SEQ ID NO: 36 | SEQ ID NO: 41 |
| AB6 | SEQ ID NO: 30 | SEQ ID NO: 35 | SEQ ID NO: 40 |
| AB7 | SEQ ID NO: 28 | SEQ ID NO: 35 | SEQ ID NO: 40 |
| AB8 | SEQ ID NO: 32 | SEQ ID NO: 37 | SEQ ID NO: 42 |

TABLE 2
Amino acid sequences of Variable Heavy (VHs)
and variable light (VL) domains from
exemplary anti-CD28 antibodies.

| | Variable Heavy | Variable Light |
|---|---|---|
| Exemplary | Domain | Domain |
| Antibody | (SEQ ID NO) | (SEQ ID NO) |
| AB1 | SEQ ID NO: 1 | SEQ ID NO: 7 |
| AB2 | SEQ ID NO: 2 | SEQ ID NO: 8 |
| AB3 | SEQ ID NO: 3 | SEQ ID NO: 9 |
| AB4 | SEQ ID NO: 4 | SEQ ID NO: 9 |
| AB5 | SEQ ID NO: 4 | SEQ ID NO: 10 |
| AB6 | SEQ ID NO: 3 | SEQ ID NO: 9 |
| AB7 | SEQ ID NO: 5 | SEQ ID NO: 11 |
| AB8 | SEQ ID NO: 6 | SEQ ID NO: 12 |

In some embodiments, any of the antibody or antigen binding fragments thereof of the disclosure comprises: (1) an CDRH1 having a sequence of any one of SEQ ID NOS: 13, 14, 15, 16 or 17 or a variant thereof that has a sequence having one amino acid substitution relative to a sequence of any one of SEQ ID NOS: 13, 14, 15, 16 or 17; (2) an CDRH2 having a sequence of any one of SEQ ID NOS: 18, 19, 20, 21 or 22 or a variant thereof that has a sequence having one, two, or three amino acid substitutions relative to a sequence of any one of SEQ ID NOS: 18, 19, 20, 21 or 22; (3) an CDRH3 having a sequence of any one of SEQ ID NOS: 23, 24, 25, 26 or 27, or a variant thereof that has a sequence having one or two amino acid substitutions relative to a sequence of any one of SEQ ID NOS: 23, 24, 25, 26 or 27; (4) a CDRL1 having a sequence of any one of SEQ ID NOS: 28, 29, 30, 31 or 32 or a variant thereof that has a sequence having one or two amino acid substitutions relative to a sequence of any one of SEQ ID NOS: 28, 29, 30, 31 or 32; (5) a CDRL2 having a sequence of any one of SEQ ID NOS: 33, 34, 35, 36 or 37 or a variant thereof that has a sequence having one substitution relative to a sequence of any one of SEQ ID NOS: 33, 34, 35, 36 or 37; and (6) a CDRL3 having a sequence of any one of SEQ ID NOS: 38, 39, 40, 41 or 42, or a variant thereof that has a sequence having one or two amino acid substitutions relative to the sequence of SEQ ID NO: 38, 39, 40, 41 or 42.

In some embodiments, the antibody or antigen binding fragment thereof of any of the embodiments disclosed herein contains an immunoglobulin heavy chain variable domain with the amino acid sequence set forth in any of SEQ ID NOS: 1, 2, 3, 4, 5 or 6, or a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity to any of SEQ ID NOS: 1, 2, 3, 4, 5 or 6. In some of any embodiments, the immunoglobulin heavy chain variable domain comprises the amino acid sequence set forth in any of SEQ ID NOS: 1, 2, 3, 4, 5 or 6.

In some embodiments, the antibody or antigen binding fragment thereof of any of the embodiments disclosed herein contains an immunoglobulin light chain variable domain comprises the amino acid sequence set forth in any of SEQ ID NOS: 7, 8, 9, 10, 11 or 12, or a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity to any of SEQ ID NOS: 7, 8, 9, 10, 11 or 12. In some of any embodiments, the immunoglobulin light chain variable domain comprises the amino acid sequence set forth in any of SEQ ID NOS: 7, 8, 9, 10, 11 or 12.

### AB1

In particular embodiments, an antibody or antigen binding fragment thereof of the disclosure can comprise: (1) a CDRH1 having the sequence of SEQ ID NO:13 or a sequence having one amino acid substitution relative to the sequence of SEQ ID NO:13; (2) a CDRH2 having the sequence of SEQ ID NO: 18 or a sequence having one, two or three amino acid substitutions relative to the sequence of SEQ ID NO:18; (3) a CDRH3 having the sequence of SEQ ID NO:23 or a sequence having one or two amino acid substitutions relative to the sequence of SEQ ID NO:23. In some embodiments, an antibody or antigen binding fragment thereof of the disclosure can comprise: (4) a CDRL1 having the sequence of SEQ ID NO:28 or a sequence having one or two amino acid substitutions relative to the sequence of SEQ ID NO: 28; (5) a CDRL2 having the sequence of SEQ ID NO:33 or a sequence having one amino acid substitution relative to the sequence of SEQ ID NO:33; and (6) a CDRL3 having the sequence of SEQ ID NO:38 or a sequence having one or two amino acid substitutions relative to the sequence of SEQ ID NO:38.

In particular embodiments, an antibody or antigen binding fragment thereof of the disclosure can comprise: (1) a CDRH1 having the sequence of SEQ ID NO:13 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 13; (2) a CDRH2 having the sequence of SEQ ID NO: 18 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 18; (3) a CDRH3 having the sequence of SEQ ID NO:23 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:23. In some embodiments, the antibody or antigen binding fragment thereof of the disclosure can comprise: (4) a CDRL1 having the sequence of SEQ ID NO:28 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:28; (5) a CDRL2 having the sequence of SEQ ID NO:33 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:33; and (6) a CDRL3 having the sequence of SEQ ID NO:38 or a sequence of amino acids that exhibits at least 80%, 81%, 82%, 83%, more sequence identity to SEQ ID NO:38. In particular embodiments, an antibody or antigen binding fragment thereof of the disclosure can comprise: (1) an CDRH1 having the sequence of SEQ ID NO:13; (2) an CDRH2 having the sequence of SEQ ID NO:18; (3) an CDRH3 having the sequence of SEQ ID NO:23; (4) a CDRL1 having the sequence of SEQ ID NO:28; (5) a CDRL2 having the sequence of SEQ ID NO:33; and (6) a CDRL3 having the sequence of SEQ ID NO:38.

In some embodiments, the antibody or antigen binding fragment thereof of any of the embodiments disclosed herein can include an immunoglobulin heavy chain variable domain comprising the amino acid sequence set forth in SEQ ID NO: 1 or a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity to SEQ ID NO:1. In some embodiments, the immunoglobulin heavy chain variable domain comprises the amino acid sequence set forth in SEQ ID NO: 1. In some embodiments, the immunoglobulin light chain variable domain comprises the amino acid sequence set forth in SEQ ID NO: 7, or a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity to SEQ ID NO: 7. In some embodiments, the immunoglobulin light chain variable domain comprises the amino acid sequence set forth in SEQ ID NO: 7.

In certain embodiments, the antibody or antigen binding fragment thereof comprises (i) a heavy chain comprising a heavy chain variable domain having the sequence of SEQ ID NO: 1 and a sequence having at least 90% (e.g., 90%, 91%, 92%, 93%, 94%, 95%, 96%, 97%, 98%, 99%, or 100%) identity to the sequence of SEQ ID NO: 1; and (ii) a light chain comprising a light chain variable domain having the sequence of SEQ ID NO:7 and a sequence having at least 90% (e.g., 90%, 91%, 92%, 93%, 94%, 95%, 96%, 97%, 98%, 99%, or 100%) identity to the sequence of SEQ ID NO:7.

In some embodiments, the antibody or antigen binding fragment thereof can comprise (1) a heavy chain variable domain having an CDRH1, an CDRH2, and an CDRH3 of SEQ ID NOS: 13, 18 and 23, respectively, and at least 90% (e.g., 90%, 91%, 92%, 93%, 94%, 95%, 96%, 97%, 98%, 99%, or 100%) identity to the sequence of SEQ ID NO: 1 and (2) a light chain variable domain having a CDRL1, a CDRL2, and a CDRL3 of SEQ ID NOS: 28, 33 and 38, respectively, and at least 90% (e.g., 90%, 91%, 92%, 93%, 94%, 95%, 96%, 97%, 98%, 99%, or 100%) identity to the sequence of SEQ ID NO:7. Such an antibody can be an IgG1, IgG2, IgG3, or IgG4.

### AB2

In particular embodiments, an antibody or antigen binding fragment thereof of the disclosure can comprise: (1) a CDRH1 having the sequence of SEQ ID NO:14 or a sequence having one amino acid substitution relative to the sequence of SEQ ID NO:14; (2) a CDRH2 having the sequence of SEQ ID NO:19 or a sequence having one, two or three amino acid substitutions relative to the sequence of SEQ ID NO:19; (3) a CDRH3 having the sequence of SEQ ID NO:24 or a sequence having one or two amino acid substitutions relative to the sequence of SEQ ID NO:24. In some embodiments, an antibody or antigen binding fragment thereof of the disclosure can comprise: (4) a CDRL1 having the sequence of SEQ ID NO:29 or a sequence having one or two amino acid substitutions relative to the sequence of SEQ ID NO: 29; (5) a CDRL2 having the sequence of SEQ ID NO:34 or a sequence having one amino acid substitution relative to the sequence of SEQ ID NO:34; and (6) a CDRL3 having the sequence of SEQ ID NO:39 or a sequence having one or two amino acid substitutions relative to the sequence of SEQ ID NO:39.

In particular embodiments, an antibody or antigen binding fragment thereof of the disclosure can comprise: (1) a CDRH1 having the sequence of SEQ ID NO:14 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 14; (2) a CDRH2 having the sequence of SEQ ID NO: 19 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 19; (3) a CDRH3 having the sequence of SEQ ID NO:24 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:24. In some embodiments, the antibody or antigen binding fragment thereof of the disclosure can comprise: (4) a CDRL1 having the sequence of SEQ ID NO:29 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:29; (5) a CDRL2 having the sequence of SEQ ID NO:34 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:34; and (6) a CDRL3 having the sequence of SEQ ID NO:39 or a sequence of amino acids that exhibits at least 80%, 81%, 82%, 83%, more sequence identity to SEQ ID NO:39. In particular embodiments, an antibody or antigen binding fragment thereof of the disclosure can comprise: (1) an CDRH1 having the sequence of SEQ ID NO:14; (2) an CDRH2 having the sequence of SEQ ID NO:19; (3) an CDRH3 having the sequence of SEQ ID NO:24; (4) a CDRL1 having the sequence of SEQ ID NO:29; (5) a CDRL2 having the sequence of SEQ ID NO:34; and (6) a CDRL3 having the sequence of SEQ ID NO:39.

In some embodiments, the antibody or antigen binding fragment thereof of any of the embodiments disclosed herein can include an immunoglobulin heavy chain variable domain comprising the amino acid sequence set forth in SEQ ID NO: 2 or a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity to SEQ ID NO:2. In some embodiments, the immunoglobulin heavy chain variable domain comprises the amino acid sequence set forth in SEQ ID NO: 2. In some embodiments, the immunoglobulin light chain variable domain comprises the amino acid sequence set forth in SEQ ID NO: 8, or a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity to SEQ ID NO: 8. In some embodiments, the immunoglobulin light chain variable domain comprises the amino acid sequence set forth in SEQ ID NO: 8.

In certain embodiments, the antibody or antigen binding fragment thereof comprises (i) a heavy chain comprising a heavy chain variable domain having the sequence of SEQ ID NO: 2 and a sequence having at least 90% (e.g., 90%, 91%, 92%, 93%, 94%, 95%, 96%, 97%, 98%, 99%, or 100%) identity to the sequence of SEQ ID NO:2; and (ii) a light chain comprising a light chain variable domain having the sequence of SEQ ID NO:8 and a sequence having at least 90% (e.g., 90%, 91%, 92%, 93%, 94%, 95%, 96%, 97%, 98%, 99%, or 100%) identity to the sequence of SEQ ID NO:8.

In some embodiments, the antibody or antigen binding fragment thereof can comprise (1) a heavy chain variable domain having an CDRH1, an CDRH2, and an CDRH3 of SEQ ID NOS: 14, 19 and 24, respectively, and at least 90% (e.g., 90%, 91%, 92%, 93%, 94%, 95%, 96%, 97%, 98%, 99%, or 100%) identity to the sequence of SEQ ID NO: 2, and (2) a light chain variable domain having a CDRL1, a CDRL2, and a CDRL3 of SEQ ID NOS: 29, 34 and 39, respectively, and at least 90% (e.g., 90%, 91%, 92%, 93%, 94%, 95%, 96%, 97%, 98%, 99%, or 100%) identity to the sequence of SEQ ID NO:8. Such an antibody can be an IgG1, IgG2, IgG3, or IgG4.

### AB3

In particular embodiments, an antibody or antigen binding fragment thereof of the disclosure can comprise: (1) a CDRH1 having the sequence of SEQ ID NO:15 or a sequence having one amino acid substitution relative to the sequence of SEQ ID NO:15; (2) a CDRH2 having the sequence of SEQ ID NO:20 or a sequence having one, two or three amino acid substitutions relative to the sequence of SEQ ID NO:20; (3) a CDRH3 having the sequence of SEQ ID NO:25 or a sequence having one or two amino acid substitutions relative to the sequence of SEQ ID NO:25. In some embodiments, an antibody or antigen binding fragment thereof of the disclosure can comprise: (4) a CDRL1 having the sequence of SEQ ID NO:30 or a sequence having one or two amino acid substitutions relative to the sequence of SEQ ID NO: 30; (5) a CDRL2 having the sequence of SEQ ID NO:35 or a sequence having one amino acid substitution relative to the sequence of SEQ ID NO:35; and (6) a CDRL3 having the sequence of SEQ ID NO:40 or a sequence having one or two amino acid substitutions relative to the sequence of SEQ ID NO:40.

In particular embodiments, an antibody or antigen binding fragment thereof of the disclosure can comprise: (1) a CDRH1 having the sequence of SEQ ID NO:15 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 15; (2) a CDRH2 having the sequence of SEQ ID NO:20 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:20; (3) a CDRH3 having the sequence of SEQ ID NO:25 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:25. In some embodiments, the antibody or antigen binding fragment thereof of the disclosure can comprise: (4) a CDRL1 having the sequence of SEQ ID NO:30 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:30; (5) a CDRL2 having the sequence of SEQ ID NO:35 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:35; and (6) a CDRL3 having the sequence binding fragment thereof of the disclosure can comprise: (1) an CDRH1 having the sequence of SEQ ID NO:15; (2) an CDRH2 having the sequence of SEQ ID NO: 20; (3) an CDRH3 having the sequence of SEQ ID NO:25; (4) a CDRL1 having the sequence of SEQ ID NO:30; (5) a CDRL2 having the sequence of SEQ ID NO:35; and (6) a CDRL3 having the sequence of SEQ ID NO:40.

In some embodiments, the antibody or antigen binding fragment thereof of any of the embodiments disclosed herein can include an immunoglobulin heavy chain variable domain comprising the amino acid sequence set forth in SEQ ID NO: 3 or a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity to SEQ ID NO:3. In some embodiments, the immunoglobulin heavy chain variable domain comprises the amino acid sequence set forth in SEQ ID NO: 3. In some embodiments, the immunoglobulin light chain variable domain comprises the amino acid sequence set forth in SEQ ID NO: 9, or a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity to SEQ ID NO: 9. In some embodiments, the immunoglobulin light chain variable domain comprises the amino acid sequence set forth in SEQ ID NO: 9.

In certain embodiments, the antibody or antigen binding fragment thereof comprises (i) a heavy chain comprising a heavy chain variable domain having the sequence of SEQ ID NO: 3 and a sequence having at least 90% (e.g., 90%, 91%, 92%, 93%, 94%, 95%, 96%, 97%, 98%, 99%, or 100%) identity to the sequence of SEQ ID NO:3; and (ii) a light chain comprising a light chain variable domain having the sequence of SEQ ID NO:9 and a sequence having at least 90% (e.g., 90%, 91%, 92%, 93%, 94%, 95%, 96%, 97%, 98%, 99%, or 100%) identity to the sequence of SEQ ID NO:9.

In some embodiments, the antibody or antigen binding fragment thereof can comprise (1) a heavy chain variable domain having an CDRH1, an CDRH2, and an CDRH3 of SEQ ID NOS: 15, 20 and 25, respectively, and at least 90% (e.g., 90%, 91%, 92%, 93%, 94%, 95%, 96%, 97%, 98%, 99%, or 100%) identity to the sequence of SEQ ID NO: 3, and (2) a light chain variable domain having a CDRL1, a CDRL2, and a CDRL3 of SEQ ID NOS: 30, 35 and 40, respectively, and at least 90% (e.g., 90%, 91%, 92%, 93%, 94%, 95%, 96%, 97%, 98%, 99%, or 100%) identity to the sequence of SEQ ID NO:9. Such an antibody can be an IgG1, IgG2, IgG3, or IgG4.

### AB4

In particular embodiments, an antibody or antigen binding fragment thereof of the disclosure can comprise: (1) a CDRH1 having the sequence of SEQ ID NO:16 or a sequence having one amino acid substitution relative to the sequence of SEQ ID NO:16; (2) a CDRH2 having the sequence of SEQ ID NO:21 or a sequence having one, two or three amino acid substitutions relative to the sequence of SEQ ID NO:21; (3) a CDRH3 having the sequence of SEQ ID NO:26 or a sequence having one or two amino acid substitutions relative to the sequence of SEQ ID NO:26. In some embodiments, an antibody or antigen binding fragment thereof of the disclosure can comprise: (4) a CDRL1 having the sequence of SEQ ID NO:30 or a sequence having one or two amino acid substitutions relative to the sequence of SEQ ID NO: 30; (5) a CDRL2 having the sequence of SEQ ID NO:35 or a sequence having one amino acid substitution relative to the sequence of SEQ ID NO:35; and (6) a CDRL3 having the sequence of SEQ ID NO:40 or a sequence having one or two amino acid substitutions relative to the sequence of SEQ ID NO:40.

In particular embodiments, an antibody or antigen binding fragment thereof of the disclosure can comprise: (1) a CDRH1 having the sequence of SEQ ID NO:16 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 16; (2) a CDRH2 having the sequence of SEQ ID NO:21 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:21; (3) a CDRH3 having the sequence of SEQ ID NO:26 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:26. In some embodiments, the antibody or antigen binding fragment thereof of the disclosure can comprise: (4) a CDRL1 having the sequence of SEQ ID NO:30 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:30; (5) a CDRL2 having the sequence of SEQ ID NO:35 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:35; and (6) a CDRL3 having the sequence binding fragment thereof of the disclosure can comprise: (1) an CDRH1 having the sequence of SEQ ID NO:16; (2) an CDRH2 having the sequence of SEQ ID NO:21; (3) an CDRH3 having the sequence of SEQ ID NO:26; (4) a CDRL1 having the sequence of SEQ ID NO:30; (5) a CDRL2 having the sequence of SEQ ID NO:35; and (6) a CDRL3 having the sequence of SEQ ID NO:40.

In some embodiments, the antibody or antigen binding fragment thereof of any of the embodiments disclosed herein can include an immunoglobulin heavy chain variable domain comprising the amino acid sequence set forth in SEQ ID NO: 4 or a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity to SEQ ID NO:4. In some embodiments, the immunoglobulin heavy chain variable domain comprises the amino acid sequence set forth in SEQ ID NO: 4. In some embodiments, the immunoglobulin light chain variable domain comprises the amino acid sequence set forth in SEQ ID NO: 9, or a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity to SEQ ID NO:9. In some embodiments, the immunoglobulin light chain variable domain comprises the amino acid sequence set forth in SEQ ID NO: 9.

In certain embodiments, the antibody or antigen binding fragment thereof comprises (i) a heavy chain comprising a heavy chain variable domain having the sequence of SEQ ID NO: 4 and a sequence having at least 90% (e.g., 90%, 91%, 92%, 93%, 94%, 95%, 96%, 97%, 98%, 99%, or 100%) identity to the sequence of SEQ ID NO:4; and (ii) a light chain comprising a light chain variable domain having the sequence of SEQ ID NO:9 and a sequence having at least 90% (e.g., 90%, 91%, 92%, 93%, 94%, 95%, 96%, 97%, 98%, 99%, or 100%) identity to the sequence of SEQ ID NO:9.

In some embodiments, the antibody or antigen binding fragment thereof can comprise (1) a heavy chain variable domain having an CDRH1, an CDRH2, and an CDRH3 of SEQ ID NOS: 16, 21 and 26, respectively, and at least 90% (e.g., 90%, 91%, 92%, 93%, 94%, 95%, 96%, 97%, 98%, 99%, or 100%) identity to the sequence of SEQ ID NO: 4, and (2) a light chain variable domain having a CDRL1, a CDRL2, and a CDRL3 of SEQ ID NOS: 30, 35 and 40, respectively, and at least 90% (e.g., 90%, 91%, 92%, 93%, 94%, 95%, 96%, 97%, 98%, 99%, or 100%) identity to the sequence of SEQ ID NO:9. Such an antibody can be an IgG1, IgG2, IgG3, or IgG4.

### AB5

In particular embodiments, an antibody or antigen binding fragment thereof of the disclosure can comprise: (1) a CDRH1 having the sequence of SEQ ID NO:16 or a sequence having one amino acid substitution relative to the sequence of SEQ ID NO:16; (2) a CDRH2 having the sequence of SEQ ID NO:21 or a sequence having one, two or three amino acid substitutions relative to the sequence of SEQ ID NO:21; (3) a CDRH3 having the sequence of SEQ ID NO:26 or a sequence having one or two amino acid substitutions relative to the sequence of SEQ ID NO:26. In some embodiments, an antibody or antigen binding fragment thereof of the disclosure can comprise: (4) a CDRL1 having the sequence of SEQ ID NO:31 or a sequence having one or two amino acid substitutions relative to the sequence of SEQ ID NO: 31; (5) a CDRL2 having the sequence of SEQ ID NO:36 or a sequence having one amino acid substitution relative to the sequence of SEQ ID NO:36; and (6) a CDRL3 having the sequence of SEQ ID NO:41 or a sequence having one or two amino acid substitutions relative to the sequence of SEQ ID NO:41.

In particular embodiments, an antibody or antigen binding fragment thereof of the disclosure can comprise: (1) a CDRH1 having the sequence of SEQ ID NO:16 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 16; (2) a CDRH2 having the sequence of SEQ ID NO:21 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:21; (3) a CDRH3 having the sequence of SEQ ID NO:26 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:26. In some embodiments, the antibody or antigen binding fragment thereof of the disclosure can comprise: (4) a CDRL1 having the sequence of SEQ ID NO:31 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:31; (5) a CDRL2 having the sequence of SEQ ID NO:36 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:36; and (6) a CDRL3 having the sequence of SEQ ID NO:41 or a sequence of amino acids that exhibits at least 80%, 81%, 82%, 83%, more sequence identity to SEQ ID NO:41. In particular embodiments, an antibody or antigen binding fragment thereof of the disclosure can comprise: (1) an CDRH1 having the sequence of SEQ ID NO:16; (2) an CDRH2 having the sequence of SEQ ID NO:21; (3) an CDRH3 having the sequence of SEQ ID NO:26; (4) a CDRL1 having the sequence of SEQ ID NO:31; (5) a CDRL2 having the sequence of SEQ ID NO:36; and (6) a CDRL3 having the sequence of SEQ ID NO:41.

In some embodiments, the antibody or antigen binding fragment thereof of any of the embodiments disclosed herein can include an immunoglobulin heavy chain variable domain comprising the amino acid sequence set forth in SEQ ID NO: 4 or a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity to SEQ ID NO:4. In some embodiments, the immunoglobulin heavy chain variable domain comprises the amino acid sequence set forth in SEQ ID NO: 4. In some embodiments, the immunoglobulin light chain variable domain comprises the amino acid sequence set forth in SEQ ID NO: 10, or a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity to SEQ ID NO:10. In some embodiments, the immunoglobulin light chain variable domain comprises the amino acid sequence set forth in SEQ ID NO: 10.

In certain embodiments, the antibody or antigen binding fragment thereof comprises (i) a heavy chain comprising a heavy chain variable domain having the sequence of SEQ ID NO: 4 and a sequence having at least 90% (e.g., 90%, 91%, 92%, 93%, 94%, 95%, 96%, 97%, 98%, 99%, or 100%) identity to the sequence of SEQ ID NO:4; and (ii) a light chain comprising a light chain variable domain having the sequence of SEQ ID NO: 10 and a sequence having at least 90% (e.g., 90%, 91%, 92%, 93%, 94%, 95%, 96%, 97%, 98%, 99%, or 100%) identity to the sequence of SEQ ID NO:10.

In some embodiments, the antibody or antigen binding fragment thereof can comprise (1) a heavy chain variable domain having an CDRH1, an CDRH2, and an CDRH3 of SEQ ID NOS: 16, 21 and 26, respectively, and at least 90% (e.g., 90%, 91%, 92%, 93%, 94%, 95%, 96%, 97%, 98%, 99%, or 100%) identity to the sequence of SEQ ID NO: 4, and (2) a light chain variable domain having a CDRL1, a CDRL2, and a CDRL3 of SEQ ID NOS: 31, 36 and 41, respectively, and at least 90% (e.g., 90%, 91%, 92%, 93%, 94%, 95%, 96%, 97%, 98%, 99%, or 100%) identity to the sequence of SEQ ID NO: 10. Such an antibody can be an IgG1, IgG2, IgG3, or IgG4.

### AB6

In particular embodiments, an antibody or antigen binding fragment thereof of the disclosure can comprise: (1) a CDRH1 having the sequence of SEQ ID NO:15 or a sequence having one amino acid substitution relative to the sequence of SEQ ID NO:15; (2) a CDRH2 having the sequence of SEQ ID NO:20 or a sequence having one, two or three amino acid substitutions relative to the sequence of SEQ ID NO:20; (3) a CDRH3 having the sequence of SEQ ID NO:25 or a sequence having one or two amino acid substitutions relative to the sequence of SEQ ID NO:25. In some embodiments, an antibody or antigen binding fragment thereof of the disclosure can comprise: (4) a CDRL1 having the sequence of SEQ ID NO:30 or a sequence having one or two amino acid substitutions relative to the sequence of SEQ ID NO: 30; (5) a CDRL2 having the sequence of SEQ ID NO:35 or a sequence having one amino acid substitution relative to the sequence of SEQ ID NO:35; and (6) a CDRL3 having the sequence of SEQ ID NO:40 or a sequence having one or two amino acid substitutions relative to the sequence of SEQ ID NO:40.

In particular embodiments, an antibody or antigen binding fragment thereof of the disclosure can comprise: (1) a CDRH1 having the sequence of SEQ ID NO:15 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 15; (2) a CDRH2 having the sequence of SEQ ID NO:20 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:20; (3) a CDRH3 having the sequence of SEQ ID NO:25 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:25. In some embodiments, the antibody or antigen binding fragment thereof of the disclosure can comprise: (4) a CDRL1 having the sequence of SEQ ID NO:30 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:30; (5) a CDRL2 having the sequence of SEQ ID NO:35 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:35; and (6) a CDRL3 having the sequence binding fragment thereof of the disclosure can comprise: (1) an CDRH1 having the sequence of SEQ ID NO:15; (2) an CDRH2 having the sequence of SEQ ID NO:20; (3) an CDRH3 having the sequence of SEQ ID NO:25; (4) a CDRL1 having the sequence of SEQ ID NO:30; (5) a CDRL2 having the sequence of SEQ ID NO:35; and (6) a CDRL3 having the sequence of SEQ ID NO:40.

In some embodiments, the antibody or antigen binding fragment thereof of any of the embodiments disclosed herein can include an immunoglobulin heavy chain variable domain comprising the amino acid sequence set forth in SEQ ID NO: 3 or a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity to SEQ ID NO:3. In some embodiments, the immunoglobulin heavy chain variable domain comprises the amino acid sequence set forth in SEQ ID NO: 3. In some embodiments, the immunoglobulin light chain variable domain comprises the amino acid sequence set forth in SEQ ID NO: 9, or a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity to SEQ ID NO:9. In some embodiments, the immunoglobulin light chain variable domain comprises the amino acid sequence set forth in SEQ ID NO: 9.

In certain embodiments, the antibody or antigen binding fragment thereof comprises (i) a heavy chain comprising a heavy chain variable domain having the sequence of SEQ ID NO: 3 and a sequence having at least 90% (e.g., 90%, 91%, 92%, 93%, 94%, 95%, 96%, 97%, 98%, 99%, or 100%) identity to the sequence of SEQ ID NO:3; and (ii) a light chain comprising a light chain variable domain having the sequence of SEQ ID NO:9 and a sequence having at least 90% (e.g., 90%, 91%, 92%, 93%, 94%, 95%, 96%, 97%, 98%, 99%, or 100%) identity to the sequence of SEQ ID NO:9.

In some embodiments, the antibody or antigen binding fragment thereof can comprise (1) a heavy chain variable domain having an CDRH1, an CDRH2, and an CDRH3 of SEQ ID NOS: 15, 20 and 25, respectively, and at least 90% (e.g., 90%, 91%, 92%, 93%, 94%, 95%, 96%, 97%, 98%, 99%, or 100%) identity to the sequence of SEQ ID NO: 3, and (2) a light chain variable domain having a CDRL1, a CDRL2, and a CDRL3 of SEQ ID NOS: 30, 35 and 40, respectively, and at least 90% (e.g., 90%, 91%, 92%, 93%, 94%, 95%, 96%, 97%, 98%, 99%, or 100%) identity to the sequence of SEQ ID NO:9. Such an antibody can be an IgG1, IgG2, IgG3, or IgG4.

### AB7

In particular embodiments, an antibody or antigen binding fragment thereof of the disclosure can comprise: (1) a CDRH1 having the sequence of SEQ ID NO:15 or a sequence having one amino acid substitution relative to the sequence of SEQ ID NO:15; (2) a CDRH2 having the sequence of SEQ ID NO:20 or a sequence having one, two or three amino acid substitutions relative to the sequence of SEQ ID NO:20; (3) a CDRH3 having the sequence of SEQ ID NO:25 or a sequence having one or two amino acid substitutions relative to the sequence of SEQ ID NO:25. In some embodiments, an antibody or antigen binding fragment thereof of the disclosure can comprise: (4) a CDRL1 having the sequence of SEQ ID NO:28 or a sequence having one or two amino acid substitutions relative to the sequence of SEQ ID NO: 28; (5) a CDRL2 having the sequence of SEQ ID NO:35 or a sequence having one amino acid substitution relative to the sequence of SEQ ID NO:35; and (6) a CDRL3 having the sequence of SEQ ID NO:40 or a sequence having one or two amino acid substitutions relative to the sequence of SEQ ID NO:40.

In particular embodiments, an antibody or antigen binding fragment thereof of the disclosure can comprise: (1) a CDRH1 having the sequence of SEQ ID NO:15 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 15; (2) a CDRH2 having the sequence of SEQ ID NO:20 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:20; (3) a CDRH3 having the sequence of SEQ ID NO:25 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:25. In some embodiments, the antibody or antigen binding fragment thereof of the disclosure can comprise: (4) a CDRL1 having the sequence of SEQ ID NO:28 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:28; (5) a CDRL2 having the sequence of SEQ ID NO:35 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:35; and (6) a CDRL3 having the sequence binding fragment thereof of the disclosure can comprise: (1) an CDRH1 having the sequence of SEQ ID NO:15; (2) an CDRH2 having the sequence of SEQ ID NO:20; (3) an CDRH3 having the sequence of SEQ ID NO:25; (4) a CDRL1 having the sequence of SEQ ID NO:28; (5) a CDRL2 having the sequence of SEQ ID NO:35; and (6) a CDRL3 having the sequence of SEQ ID NO:40.

In some embodiments, the antibody or antigen binding fragment thereof of any of the embodiments disclosed herein can include an immunoglobulin heavy chain variable domain comprising the amino acid sequence set forth in SEQ ID NO: 5 or a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity to SEQ ID NO:5. In some embodiments, the immunoglobulin heavy chain variable domain comprises the amino acid sequence set forth in SEQ ID NO: 5. In some embodiments, the immunoglobulin light chain variable domain comprises the amino acid sequence set forth in SEQ ID NO: 11, or a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity to SEQ ID NO:11. In some embodiments, the immunoglobulin light chain variable domain comprises the amino acid sequence set forth in SEQ ID NO: 11.

In certain embodiments, the antibody or antigen binding fragment thereof comprises (i) a heavy chain comprising a heavy chain variable domain having the sequence of SEQ ID NO: 5 and a sequence having at least 90% (e.g., 90%, 91%, 92%, 93%, 94%, 95%, 96%, 97%, 98%, 99%, or 100%) identity to the sequence of SEQ ID NO:5; and (ii) a light chain comprising a light chain variable domain having the sequence of SEQ ID NO: 11 and a sequence having at least 90% (e.g., 90%, 91%, 92%, 93%, 94%, 95%, 96%, 97%, 98%, 99%, or 100%) identity to the sequence of SEQ ID NO:11.

In some embodiments, the antibody or antigen binding fragment thereof can comprise (1) a heavy chain variable domain having an CDRH1, an CDRH2, and an CDRH3 of SEQ ID NOS: 15, 20 and 25, respectively, and at least 90% (e.g., 90%, 91%, 92%, 93%, 94%, 95%, 96%, 97%, 98%, 99%, or 100%) identity to the sequence of SEQ ID NO: 5, and (2) a light chain variable domain having a CDRL1, a CDRL2, and a CDRL3 of SEQ ID NOS: 28, 35 and 40, respectively, and at least 90% (e.g., 90%, 91%, 92%, 93%, 94%, 95%, 96%, 97%, 98%, 99%, or 100%) identity to the sequence of SEQ ID NO: 11. Such an antibody can be an IgG1, IgG2, IgG3, or IgG4.

### AB8

In particular embodiments, an antibody or antigen binding fragment thereof of the disclosure can comprise: (1) a CDRH1 having the sequence of SEQ ID NO:17 or a sequence having one amino acid substitution relative to the sequence of SEQ ID NO:17; (2) a CDRH2 having the sequence of SEQ ID NO:22 or a sequence having one, two or three amino acid substitutions relative to the sequence of SEQ ID NO:22; (3) a CDRH3 having the sequence of SEQ ID NO:27 or a sequence having one or two amino acid substitutions relative to the sequence of SEQ ID NO:27. In some embodiments, an antibody or antigen binding fragment thereof of the disclosure can comprise: (4) a CDRL1 having the sequence of SEQ ID NO:32 or a sequence having one or two amino acid substitutions relative to the sequence of SEQ ID NO: 32; (5) a CDRL2 having the sequence of SEQ ID NO:37 or a sequence having one amino acid substitution relative to the sequence of SEQ ID NO:37; and (6) a CDRL3 having the sequence of SEQ ID NO:42 or a sequence having one or two amino acid substitutions relative to the sequence of SEQ ID NO:42.

In particular embodiments, an antibody or antigen binding fragment thereof of the disclosure can comprise: (1) a CDRH1 having the sequence of SEQ ID NO: 17 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 17; (2) a CDRH2 having the sequence of SEQ ID NO:22 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:22; (3) a CDRH3 having the sequence of SEQ ID NO:27 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:27. In some embodiments, the antibody or antigen binding fragment thereof of the disclosure can comprise: (4) a CDRL1 having the sequence of SEQ ID NO:32 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:32; (5) a CDRL2 having the sequence of SEQ ID NO:37 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:37; and (6) a CDRL3 having the sequence of SEQ ID NO:42 or a sequence of amino acids that exhibits at least 80%, 81%, 82%, 83%, more sequence identity to SEQ ID NO:42. In particular embodiments, an antibody or antigen binding fragment thereof of the disclosure can comprise: (1) an CDRH1 having the sequence of SEQ ID NO:17; (2) an CDRH2 having the sequence of SEQ ID NO:22; (3) an CDRH3 having the sequence of SEQ ID NO:27; (4) a CDRL1 having the sequence of SEQ ID NO:32; (5) a CDRL2 having the sequence of SEQ ID NO:37; and (6) a CDRL3 having the sequence of SEQ ID NO:42.

In some embodiments, the antibody or antigen binding fragment thereof of any of the embodiments disclosed herein can include an immunoglobulin heavy chain variable domain comprising the amino acid sequence set forth in SEQ ID NO: 6 or a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity to SEQ ID NO:6. In some embodiments, the immunoglobulin heavy chain variable domain comprises the amino acid sequence set forth in SEQ ID NO: 6. In some embodiments, the immunoglobulin light chain variable domain comprises the amino acid sequence set forth in SEQ ID NO: 12, or a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity to SEQ ID NO:12. In some embodiments, the immunoglobulin light chain variable domain comprises the amino acid sequence set forth in SEQ ID NO: 12.

In certain embodiments, the antibody or antigen binding fragment thereof comprises (i) a heavy chain comprising a heavy chain variable domain having the sequence of SEQ ID NO: 6 and a sequence having at least 90% (e.g., 90%, 91%, 92%, 93%, 94%, 95%, 96%, 97%, 98%, 99%, or 100%) identity to the sequence of SEQ ID NO:6; and (ii) a light chain comprising a light chain variable domain having the sequence of SEQ ID NO: 12 and a sequence having at least 90% (e.g., 90%, 91%, 92%, 93%, 94%, 95%, 96%, 97%, 98%, 99%, or 100%) identity to the sequence of SEQ ID NO:12.

In some embodiments, the antibody or antigen binding fragment thereof can comprise (1) a heavy chain variable domain having an CDRH1, an CDRH2, and an CDRH3 of SEQ ID NOS: 17, 22 and 27, respectively, and at least 90% (e.g., 90%, 91%, 92%, 93%, 94%, 95%, 96%, 97%, 98%, 99%, or 100%) identity to the sequence of SEQ ID NO: 6, and (2) a light chain variable domain having a CDRL1, a CDRL2, and a CDRL3 of SEQ ID NOS: 32, 37 and 42, respectively, and at least 90% (e.g., 90%, 91%, 92%, 93%, 94%, 95%, 96%, 97%, 98%, 99%, or 100%) identity to the sequence of SEQ ID NO:12. Such an antibody can be an IgG1, IgG2, IgG3, or IgG4.

### III. Fc Polypeptide

In some embodiments, any of the antibodies or antigen binding fragments thereof provided herein can comprise a fragment crystallizable region (Fc region), also referred to as an Fc polypeptide herein. An Fc polypeptide is part of each of the two heavy chains in the antibody and can interact with certain cell surface receptors and certain components of the complement system. An Fc polypeptide typically includes the CH2 domain and the CH3 domain, which are immunoglobulin constant region domain polypeptides. In some embodiments, the Fc polypeptide in an antibody described herein can be a wild-type Fc polypeptide, e.g., a human IgG1 Fc polypeptide. In certain embodiments, an antibody described herein can comprise a wild-type Fc polypeptide having the sequence of SEQ ID NO: 85:
APELLGGPSVFLFPPKPKDTLMISRTPEVTCVVVDVSHEDPEVKFNWYVDGVEVHNA KTKPREEQYNSTYRVVSVLTVLHQDWLNGKEYKCKVSNKALPAPIEKTISKAKGQP REPQVYTLPPSRDELTKNQVSLTCLVKGFYPSDIA VEWESNGQPENNYKTTPPVLDS DGSFFLYSKLTVDKSRWQQGNVFSCSVMHEALHNHYTQKSLSLSPGK. In other embodiments, an antibody described herein can comprise a variant of the wild-type Fc polypeptide that has at least 90% (e.g., 90%, 91%, 92%, 93%, 94%, 95%, 96%, 97%, 98%, 99%, or 99.5%) identity to the sequence of a wild-type Fc polypeptide (e.g., SEQ ID NO:85) and at least one amino acid substitution relative to the sequence of a wild-type Fc polypeptide (e.g., SEQ ID NO:85).

In some embodiments, an Fc polypeptide includes one or more modifications (e.g., one or more amino acid substitutions, insertions, or deletions relative to a comparable wild-type Fc region). Antibodies comprising modified Fc polypeptides typically have altered phenotypes relative to antibodies comprising wild-type Fc polypeptides. For example, antibodies comprising modified Fc polypeptides can have altered serum half-life, altered stability, altered susceptibility to cellular enzymes, and/or altered effector function (e.g., as assayed in an NK-dependent or macrophage-dependent assay).

In some embodiments, an Fc polypeptide in an antibody described herein can include amino acid substitutions that modulate effector function. In certain embodiments, an Fc polypeptide in an antibody described herein can include amino acid substitutions that reduce or eliminate effector function. Illustrative Fc polypeptide amino acid substitutions that reduce effector function include, but are not limited to, substitutions in a CH2 domain, e.g., at positions 4 and 5 (position numbering relative to the sequence of SEQ ID NO:85) (see, e.g., Lund et al., J Immunol. 147 (8): 2657-62, 1991). For example, in some embodiments, one or both Fc polypeptides in an antibody described herein can comprise L4A and L5A substitutions.

Additional Fc polypeptide amino acid substitutions that modulate an effector function include, e.g., substitution at position 99 (position numbering relative to the sequence of SEQ ID NO: 85). For example, in some embodiments, one or both Fc polypeptides in an antibody described herein can comprise P99G substitution. In certain embodiments, one or both Fc polypeptides in an antibody described herein can have L4A, L5A, and P99G substitutions.

In some embodiments, an Fc polypeptide includes one or more modifications that alter (relative to a wild-type Fc polypeptide) the Ratio of Affinities of the modified Fc polypeptide to an activating FcγR (such as FcγRIIA or FcγRIIIA) relative to an inhibiting FcγR (such as FcγRIIB):

$$Ratio of Affinities = \frac{Wild - Type to Variant Change in Affinity to Fc γ {R}_{Activating}}{Wild - Type to Variant Change in Affinity to Fc γ {R}_{Inhibiting}} .$$

Where a modified Fc polypeptide has a Ratio of Affinities greater than 1, an antibody herein may have particular use in providing a therapeutic or prophylactic treatment of a disease, disorder, or infection, or the amelioration of a symptom thereof, where an enhanced efficacy of effector cell function (e.g., ADCC) mediated by FcγR is desired, e.g., cancer or infectious disease. Where a modified Fc region has a Ratio of Affinities less than 1, an antibody herein may have particular use in providing a therapeutic or prophylactic treatment of a disease or disorder, or the amelioration of a symptom thereof, where a decreased efficacy of effector cell function mediated by FcγR is desired, e.g., autoimmune or inflammatory disorders. Table 3 lists examples of single, double, triple, quadruple, and quintuple amino acid substitutions in an Fc polypeptide that provide a Ratio of Affinities greater than 1 or less than 1 (see e.g., PCT Publication Nos. WO 04/063351; WO 06/088494; WO 07/024249; WO 06/113665; WO 07/021841; WO 07/106707; WO 2008/140603). Amino acid positions are numbered according to EU numbering scheme.

TABLE 3
Exemplary Fc amino acid substitutions

| Ratio | Single | Double | Triple | Quadruple | Quintuple |
|---|---|---|---|---|---|
| >1 | F243L | F243L & | F243L, P247L & | L234F, F243L, | L235V, |
| | D270E | R292P | N421K | R292P & Y300L | F243L, R292P, |
| | R292G | F243L & | F243L, R292P & | L235I, F243L, | Y300L & |
| | R292P | Y300L | Y300L | R292P & Y300L | P396L |
| | | F243L & | F243L, R292P & | L235Q, F243L, | L235P, F243L, |
| | | P396L | V305I | R292P & Y300L | R292P, Y300L & |
| | | D270E & | F243L, R292P & | F243L, P247L, | P396L |
| | | P396L | P396L | D270E & N421K | F243L, R292P, |
| | | R292P & | F243L, Y300L & | F243L, R255L, | V305I, Y300L & |
| | | Y300L | P396L | D270E & P396L | P396L |
| | | R292P & | P247L, D270E & | F243L, D270E, | |
| | | V305I | N421K | G316D & R416G | |
| | | R292P & | R255L, D270E & | F243L, D270E, | |
| | | P396L | P396L | K392T & P396L | |
| | | Y300L & | D270E, G316D & | F243L, D270E, | |
| | | P396L | R416G | P396L & Q419H | |
| | | P396L & | D270E, K392T & | F243L, R292P, | |
| | | Q419H | P396L | Y300L, & P396L | |
| | | | D270E, P396L & | F243L, R292P, | |
| | | | Q419H | V305I & P396L | |
| | | | V284M, R292L & | P247L, D270E, | |
| | | | K370N | Y300L & N421K | |
| | | | R292P, Y300L & | R255L, D270E, | |
| | | | P396L | R292G & P396L | |
| | | | | R255L, D270E, | |
| | | | | Y300L & P396L | |
| | | | | D270E, G316D, | |
| | | | | P396L & R416G | |
| <1 | Y300L | F243L & | F243L, R292P & | | |
| | P396L | P396L | V305I | | |
| | | P247L & | | | |
| | | N421K | | | |
| | | R255L & | | | |
| | | P396L | | | |
| | | R292P & | | | |
| | | V305I | | | |
| | | K392T & | | | |
| | | P396L | | | |
| | | P396L & | | | |
| | | Q419H | | | |

### IV. Antibodies that Competitively Bind with an Anti-CD28 Antibody

Also provided herein are antibodies that competitively bind, or are capable of competitively binding (e.g., competitor antibodies), with one or more CD28 antibodies described herein. In certain instances, an antibody (i.e., competitor antibody) may be considered to compete for binding to when the competitor binds to the same general region of as an antibody described herein. In certain instances, an antibody (i.e., competitor antibody) may be considered to compete for binding to when the competitor binds to the exact same region of as an antibody described herein (e.g., exact same peptide (linear epitope) or exact same surface amino acids (conformational epitope)). In certain instances, an antibody (i.e., competitor antibody) may be considered capable of competing for binding to when the competitor binds to the same general region of as an antibody described herein (i.e., extracellular region or leucine-rich binding domain) under suitable assay conditions. In certain instances, an antibody (i.e., competitor agent) may be considered capable of competing for binding to when the competitor binds to the exact same region of as an antibody described herein (e.g., exact same peptide (linear epitope) or exact same surface amino acids (conformational epitope)) under suitable assay conditions.

In certain instances, an antibody (i.e., competitor antibody) may be considered to compete for binding to CD28 when the competitor blocks the binding of one or more antibodies described herein to CD28, for example, under suitable assay conditions. Whether a competitor blocks the binding of one or more antibodies described herein to may be determined using a suitable competition assay or blocking assay, such as, for example, a blocking assay as described in herein. A competitor antibody may block binding of one or more antibodies described herein to in a competition or blocking assay by 50% or more (e.g., 55%, 60%, 65%, 70%, 75%, 80%, 85%, 90%, 95% or more, or 100%), and conversely, one or more antibodies described herein may block binding of the competitor antibody to in a competition or blocking assay by about 50% or more (e.g., e.g., 55%, 60%, 65%, 70%, 75%, 80%, 85%, 90%, 95% or more, or 100%).

In certain instances, an antibody (i.e., competitor antibody) may be considered to compete for binding to CD28 when the competitor binds to with a similar affinity as one or more antibodies described herein, for example, under suitable assay conditions. In some embodiments, an antibody (i.e., competitor antibody) is considered to compete for binding to when the competitor binds to with an affinity that is at least about 50% (e.g., 55%, 60%, 65%, 70%, 75%, 80%, 85%, 90%, 95%, or 100%) of the affinity of one or more antibodies described herein.

Also provided herein are antibodies that bind to, or are capable of binding to, the same epitope as one or more antibodies described herein. In particular, provided herein are antibodies that compete with one or more antibodies described herein for binding to the same epitope (e.g., same peptide (linear epitope) or same surface amino acids (conformational epitope)) on CD28. Such antibodies that bind the same epitope may be referred to as epitope competitors.

### V. Polyclonal and Monoclonal Antibodies

Polyclonal antibodies may be raised in animals (vertebrate or invertebrates, including mammals, birds and fish, including cartilaginous fish) by multiple subcutaneous (sc) or intraperitoneal (ip) injections of a relevant antigen and an adjuvant. It may be useful to conjugate the relevant antigen to a protein or other carrier that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl2, or R1N═C═NR, where R and R1 are different alkyl groups. Non-protein carriers (e.g., colloidal gold) also may be used for antibody production.

Animals can be immunized against the antigen, immunogenic conjugates, or derivatives by combining, e.g., 100 μg or 5 μg of the protein or conjugate (for rabbits or mice, respectively) with three volumes of Freund's complete adjuvant and injecting the solution intradermally at multiple sites. One month later the animals are boosted with one-fifth to one-tenth of the original amount of peptide or conjugate in Freund's complete adjuvant by subcutaneous injection at multiple sites. Seven to 14 days later the animals are bled and the serum is assayed for antibody titer. Animals are boosted until the titer plateaus. Often, the animal is boosted with the conjugate of the same antigen, but conjugated to a different protein and/or through a different cross-linking reagent. Conjugates also can be made in recombinant cell culture as protein fusions. Also, aggregating agents such as alum are suitably used to enhance the immune response.

Monoclonal antibodies may be made using a hybridoma, e.g., the hybridoma method first described by Kohler et al., Nature, 256:495 (1975), or may be made by other methods such as recombinant DNA methods (see, e.g., U.S. Pat. No. 4,816,567). In the hybridoma method, a mouse or other appropriate host animal, such as a hamster or macaque monkey, is immunized to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (see, e.g., Goding, Monoclonal Antibodies: Principles and Practice, pp. 59-103 (Academic Press, 1986)).

The hybridoma cells thus prepared are seeded and grown in a suitable culture medium that may contain one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells. Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as SP-2 or X63-Ag8-653 cells available from the American Type Culture Collection, Rockville, Md. USA. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. The binding specificity of monoclonal antibodies produced by hybridoma cells may be determined by immunoprecipitation, by an in vitro binding assay, such as radioimmunoassay (RIA) or enzyme-linked immunoabsorbant assay (ELISA), or by flow cytometric analysis of cells expressing the membrane antigen. The binding affinity of the monoclonal antibody can, for example, be determined by the Scatchard analysis of Munson et al., Anal. Biochem., 107:220 (1980).

After hybridoma cells are identified that produce antibodies of the desired specificity, affinity, and/or activity, the clones may be subcloned by limiting dilution procedures and grown by standard methods (see, e.g., Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal. The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

DNA encoding the monoclonal antibodies can be readily isolated and sequenced using conventional procedures (e.g., by using oligonucleotide probes that are capable of binding specifically to genes encoding the heavy and light chains of the monoclonal antibodies). Alternatively, cDNA may be prepared from mRNA and the cDNA then subjected to DNA sequencing. The hybridoma cells serve as a preferred source of such genomic DNA or RNA for cDNA preparation. Once isolated, the DNA may be placed into expression vectors, which are well known in the art, and which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells.

### VI. Humanization and Amino Acid Variants

General methods for humanization of antibodies are described, for example, in U.S. Pat. Nos. 5,861,155, 6,479,284, 6,407,213, 6,639,055, 6,500,931, 5,530,101, 5,585,089, 5,693,761, 5,693,762, 6,180,370, 5,714,350, 6,350,861, 5,777,085, 5,834,597, 5,882,644, 5,932,448, 6,013,256, 6,129,914, 6,210,671, 6,329,511, 5,225,539, 6,548,640, and 5,624,821. In certain embodiments, it may be desirable to generate amino acid sequence variants of these humanized antibodies, particularly where these improve the binding affinity or other biological properties (e.g., half-life) of the antibody.

In some embodiments, the antibody is a humanized antibody, i.e., an antibody that retains the reactivity of a non-human antibody while being less immunogenic in humans. This can be achieved, for instance, by retaining the non-human CDR regions and replacing the remaining parts of the antibody with their human counterparts. See, e.g., Morrison et al., PNAS USA, 81:6851-6855 (1984); Morrison and Oi, Adv. Immunol., 44:65-92 (1988); Verhoeyen et al., Science, 239:1534-1536 (1988); Padlan, Molec. Immun., 28:489-498 (1991); Padlan, Molec. Immun., 31 (3): 169-217 (1994). Techniques for humanizing antibodies are well known in the art and are described in e.g., U.S. Pat. Nos. 4,816,567; 5,530,101; 5,859,205; 5,585,089; 5,693,761; 5,693,762; 5,777,085; 6,180,370; 6,210,671; and 6,329,511; WO 87/02671; EP Patent Application 0173494; Jones et al. (1986) Nature 321:522; and Verhoyen et al. (1988) Science 239:1534. Humanized antibodies are further described in, e.g., Winter and Milstein (1991) Nature 349:293. For example, polynucleotides comprising a first sequence coding for humanized immunoglobulin framework regions and a second sequence set coding for the desired immunoglobulin complementarity determining regions can be produced synthetically or by combining appropriate cDNA and genomic DNA segments. Human constant region DNA sequences can be isolated in accordance with well-known procedures from a variety of human cells. The CDRs for producing the immunoglobulins of the present disclosure can be similarly derived from monoclonal antibodies capable of specifically binding to CD28.

Amino acid sequence variants of the antibody can be prepared by introducing appropriate nucleotide changes into the antibody DNA, or by peptide synthesis. Such variants include, for example, deletions from, and/or insertions into and/or substitutions of, residues within the amino acid sequences of the antibodies for the examples herein. Any combination of deletion, insertion, and substitution is made to arrive at the final construct, provided that the final construct possesses the desired characteristics. The amino acid changes also may alter post-translational processes of the humanized or variant antibody, such as changing the number or position of glycosylation sites.

One method for identification of certain residues or regions of the antibody that are preferred locations for mutagenesis is called “alanine scanning mutagenesis,” as described by, e.g., Cunningham and Wells, Science, 244:1081-1085 (1989). Here, a residue or group of target residues are identified (e.g., charged residues such as Arg, Asp, His, Lys, and Glu) and replaced by a neutral or negatively charged amino acid (most preferably Ala or poly-Ala) to affect the interaction of the amino acids with antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, alanine scanning or random mutagenesis is conducted at the target codon or region and the expressed antibody variants are screened for the desired activity. Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an N-terminal methionyl residue or the antibody fused to an epitope tag. Other insertional variants include the fusion of an enzyme or a polypeptide that increases the serum half-life of the antibody to the N- or C-terminus of the antibody.

Another type of variant is an amino acid substitution variant. These variants have at least one amino acid residue removed from the antibody molecule and a different residue inserted in its place. The sites of greatest interest for substitutional mutagenesis include the hypervariable regions, but FR alterations are also contemplated. Conservative substitutions are preferred, but more substantial changes may be introduced and the products may be screened. Examples of substitutions are listed below:
Ala (A): Val; Leu; Ile; Val
Arg (R): Lys; Gln; Asn; Lys
Asn (N): Gln; His; Asp, Lys; Gln; Arg
Asp (D): Glu; Asn
Cys (C): Ser; Ala
Gln (Q): Asn; Glu
Glu (E): Asp; Gln
Gly (G): Ala
His (H): Asn; Gln; Lys; Arg
Ile (I): Leu; Val; Met; Ala; Leu; Phe; Norleucine
Leu (L): Norleucine; Ile; Val; Ile; Met; Ala; Phe
Lys (K): Arg; Gln; Asn
Met (M): Leu; Phe; Ile
Phe (F): Leu; Val; Ile; Ala; Tyr
Pro (P): Ala
Ser(S): Thr
Thr (T): Ser
Trp (W): Tyr; Phe
Tyr (Y): Trp; Phe; Thr; Ser
Val (V): Ile; Leu; Met; Phe; Ala; Norleucine

Substantial modifications in the biological properties of an antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:
(1) hydrophobic: Norleucine, Met, Ala, Val, Leu, Ile;
(2) neutral hydrophilic: Cys, Ser, Thr;
(3) acidic: Asp, Glu;
(4) basic: Asn, Gln, His, Lys, Arg;
(5) residues that influence chain orientation: Gly, Pro; and
(6) aromatic: Trp, Tyr, Phe

Non-conservative substitutions will entail exchanging a member of one of the above classes for another class.

Any cysteine residue not involved in maintaining the proper conformation of the antibody also may be substituted, to improve the oxidative stability of the molecule and prevent aberrant crosslinking. Conversely, cysteine bond(s) may be added to the antibody to improve its stability (particularly where the antibody is an antigen binding fragment such as an Fv fragment).

One type of substitutional variant involves substituting one or more hypervariable region residues of a parent antibody. Generally, the resulting variant(s) selected for further development will have improved biological properties relative to the parent antibody from which they are generated. A convenient way for generating such substitutional variants is affinity maturation using phage display. Briefly, several hypervariable region sites (e.g., 6-7 sites) are mutated to generate all possible amino acid substitutions at each site. The antibody variants thus generated can be displayed in the monovalent fashion from filamentous phage particles as fusions to the gene III product of M13 packaged within each particle. The phage-displayed variants are then screened for their biological activity (e.g., binding affinity) as herein disclosed. In order to identify candidate hypervariable region sites for modification, alanine-scanning mutagenesis can be performed to identify hypervariable region residues contributing significantly to antigen binding. Alternatively, or in addition, it may be beneficial to analyze a crystal structure of the antigen-antibody complex to identify contact points between the antibody and antigen. Such contact residues and neighboring residues are candidates for substitution according to the techniques elaborated herein. Once such variants are generated, the panel of variants is subjected to screening as described herein and antibodies with superior properties in one or more relevant assays may be selected for further development.

Another type of amino acid variant of the antibody alters the original glycosylation pattern of the antibody. By altering is meant deleting one of more carbohydrate moieties found in the antibody, and/or adding one or more glycosylation sites that are not present in the antibody. Glycosylation of antibodies is typically either N-linked and/or O-linked. N-linked refers to the attachment of the carbohydrate moiety to the side chain of an asparagine residue. The tripeptide sequences asparagine-X-serine and asparagine-X-threonine, where X is any amino acid except proline, are the most common recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used. Addition of glycosylation sites to the antibody can be accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

### VII. Other Modifications

Other modifications of an antibody or antigen binding fragment thereof are contemplated. For example, technology herein also pertains to immunoconjugates comprising an antibody described herein conjugated to a cytotoxic agent such as a toxin (e.g., an enzymatically active toxin of bacterial, fungal, plant or animal origin, or fragments thereof), or a radioactive isotope (for example, a radioconjugate), or a cytotoxic drug. Such conjugates are sometimes referred to as “antibody-drug conjugates” or “ADC.” Conjugates can be made using a variety of bifunctional protein coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutaraldehyde), bis-azido compounds (such as bis-(p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene).

In some of any embodiments, any of the antibodies or antigen binding fragments thereof disclosed herein may be formulated as immunoliposomes. Liposomes containing an antibody are prepared by methods know in the art, such as described in Epstein et al., Proc. Natl. Acad. Sci. USA 82:3688 (1985); Hwang et al., Proc. Natl. Acad. Sci. USA 77:4030 (1980); and U.S. Pat. Nos. 4,485,045 and 4,544,545. Liposomes with enhanced circulation time are disclosed in U.S. Pat. No. 5,013,556. For example, liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. Fab′ fragments of an antibody provided herein can be conjugated to the liposomes as described in Martin et al., J. Biol. Chem. 257:286-288 (1982) via a disulfide interchange reaction. Another active ingredient is optionally contained within the liposome.

Enzymes or other polypeptides can be covalently bound to an antibody by techniques well known in the art such as the use of the heterobifunctional cross-linking reagents discussed above. In some embodiments, fusion proteins comprising at least the antigen binding region of an antibody provided herein linked to at least a functionally active portion of an enzyme can be constructed using recombinant DNA techniques well known in the art (see, e.g., Neuberger et al., Nature 312:604-608 (1984)).

In certain embodiments, it may be desirable to use an antigen binding fragment, rather than an intact antibody, to increase penetration of target tissues and cells, for example. In such instances, it may be desirable to modify the antigen binding fragment in order to increase its serum half-life. This may be achieved, for example, by incorporation of a salvage receptor binding epitope into the antigen binding fragment (e.g., by mutation of the appropriate region in the antigen binding fragment or by incorporating the epitope into a peptide tag that is then fused to the antigen binding fragment at either end or in the middle, e.g., by DNA or peptide synthesis; see, e.g., WO96/32478 published Oct. 17, 1996).

In some embodiments, any of the antibodies or antigen fragments thereof disclosed herein are conjugated or hybridized to an oligonucleotide. In some embodiments, the oligonucleotide includes a sample barcode sequence, a binding site for a primer and an anchor. In some embodiments, the oligonucleotide can be conjugated or hybridized to any of the detectable markers or labels disclosed herein. In some embodiments, the oligonucleotide is a polymeric sequence. In some embodiments, the terms “oligonucleotide” and “polynucleotide” are used interchangeably to refer to a single-stranded multimer of nucleotides from about 2 to about 500 nucleotides in length. In some embodiments, any of the oligonucleotides described herein can be synthetic, made enzymatically (e.g., via polymerization), or using a “split-pool” method. In some embodiments, any of the oligonucleotides described herein can include ribonucleotide monomers (i.e., can be oligoribonucleotides) and/or deoxyribonucleotide monomers (i.e., oligodeoxyribonucleotides). In some embodiments, any of the oligonucleotides described herein can include a combination of both deoxyribonucleotide monomers and ribonucleotide monomers in the oligonucleotide (e.g., random or ordered combination of deoxyribonucleotide monomers and ribonucleotide monomers). In some embodiments, the oligonucleotide can be 4 to 10, 11 to 20, 21 to 30, 31 to 40, 41 to 50, 51 to 60, 61 to 70, 71 to 80, 81 to 100, 101 to 150, 151 to 200, 201 to 250, 251 to 300, 301 to 350, 351 to 400, or 401-500 nucleotides in length. In some embodiments, any of the oligonucleotides described herein can include one or more functional moieties that are attached (e.g., covalently or non-covalently) to another structure. In some embodiments, any of the oligonucleotides described herein can include one or more detectable labels (e.g., a radioisotope or fluorophore).

In some embodiments, the anchor is a defined polymer, e.g., a polynucleotide or oligonucleotide sequence, which is designed to hybridize to a complementary oligonucleotide sequence. In some embodiments the anchor is designed for the purpose of generating a double stranded construct oligonucleotide sequence. In some embodiments, the anchor is positioned at the 3′ end of the construct oligonucleotide sequence. In other embodiments, the anchor is positioned at the 5′ end of the construct oligonucleotide sequence. Each anchor is specific for its intended complementary sequence.

In some embodiments, the sample barcode sequence is a polymer, e.g., a polynucleotide, which when it is a functional element, is specific for a single ligand. In some embodiments, the sample barcode sequence can be used for identifying a particular cell or substrate, e.g., Drop-seq microbead. In some embodiments, the sample barcode sequence can be formed of a defined sequence of DNA, RNA, modified bases or combinations of these bases, as well as any other polymer defined above. In some embodiments, the sample barcode sequence is about 2 to 4 monomeric components, e.g., nucleotide bases, in length. In other embodiments, the barcode is at least about 1 to 100 monomeric components, e.g., nucleotides, in length. Thus in various embodiments, the barcode is formed of a sequence of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 80, 91, 92, 93, 94, 95, 96, 97, 98, 99 or up to 100 monomeric components, e.g., nucleic acids. In some embodiments, the sample barcode sequence is a particular barcode that can be unique relative to other barcodes.

In some of any embodiments, the sample barcode sequences can have a variety of different formats. For example, sample barcode sequences can include polynucleotide barcodes, random nucleic acid and/or amino acid sequences, and synthetic nucleic acid and/or amino acid sequences. A sample barcode sequence can be attached to an analyte or to another moiety or structure in a reversible or irreversible manner. A sample barcode sequences can be added to, for example, a fragment of a deoxyribonucleic acid (DNA) or ribonucleic acid (RNA) sample before or during sequencing of the sample. Sample barcode sequences can allow for identification and/or quantification of individual sequencing-reads (e.g., a barcode can be or can include a unique molecular identifier or “UMI”).

Sample barcode sequences can spatially-resolve molecular components found in biological samples, for example, at single-cell resolution (e.g., a barcode can be or can include a “spatial barcode”). In some embodiments, a barcode includes both a UMI and a spatial barcode. In some embodiments, a barcode includes two or more sub-barcodes that together function as a single barcode. For example, a polynucleotide barcode can include two or more polynucleotide sequences (e.g., sub-barcodes) that are separated by one or more non-barcode sequences.

In some embodiments, the binding site for a primer is a functional component of the oligonucleotide which itself is an oligonucleotide or polynucleotide sequence that provides an annealing site for amplification of the oligonucleotide. The binding site for a primer can be formed of polymers of DNA, RNA, PNA, modified bases or combinations of these bases, or polyamides, etc. In some embodiments, the binding site for a primer is about 10 of such monomeric components, e.g., nucleotide bases, in length. In other embodiments, the binding site for a primer is at least about 5 to 100 monomeric components, e.g., nucleotides, in length. Thus in various embodiments, the binding site for a primer is formed of a sequence of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 80, 91, 92, 93, 94, 95, 96, 97, 98, 99 or up to 100 monomeric components, e.g., nucleic acids. In certain embodiments, the binding site for a primer can be a generic sequence suitable as a annealing site for a variety of amplification technologies. Amplification technologies include, but are not limited to, DNA-polymerase based amplification systems, such as polymerase chain reaction (PCR), real-time PCR, loop mediated isothermal amplification (LAMP, MALBAC), strand displacement amplification (SDA), multiple displacement amplification (MDA), recombinase polymerase amplification (RPA) and polymerization by any number of DNA polymerases (for example, T4 DNA polymerase, Sulfulobus DNA polymerase, Klenow DNA polymerase, Bst polymerase, Phi29 polymerase) and RNA-polymerase based amplification systems (such as T7-, T3-, and SP6-RNA-polymerase amplification), nucleic acid sequence based amplification (NASBA), self-sustained sequence replication (3SR), rolling circle amplification (RCA), ligase chain reaction (LCR), helicase dependent amplification (I), ramification amplification method and RNA-seq. Methods for conjugating or hybridizing an oligonucleotide can be performed in a manner set forth in WO/2018/144813, WO/2016/018960, WO/2018/089438, WO/2014/182528, WO/2018/026873, WO/2021/188838.

In some embodiments, a modification can optionally be introduced into the antibodies (e.g., within the polypeptide chain or at either the N- or C-terminal), e.g., to extend in vivo half-life, such as PEGylation or incorporation of long-chain polyethylene glycol polymers (PEG). Introduction of PEG or long chain polymers of PEG increases the effective molecular weight of the polypeptides, for example, to prevent rapid filtration into the urine. In some embodiments, a lysine residue in the sequence is conjugated to PEG directly or through a linker. Such linker can be, for example, a Glu residue or an acyl residue containing a thiol functional group for linkage to the appropriately modified PEG chain. An alternative method for introducing a PEG chain is to first introduce a Cys residue at the C-terminus or at solvent exposed residues such as replacements for Arg or Lys residues. This Cys residue is then site-specifically attached to a PEG chain containing, for example, a maleimide function. Methods for incorporating PEG or long chain polymers of PEG are known in the art (described, for example, in Veronese, F. M., et al., Drug Disc. Today 10:1451-8 (2005); Greenwald, R. B., et al., Adv. Drug Deliv. Rev. 55:217-50 (2003); Roberts, M. J., et al., Adv. Drug Deliv. Rev., 54:459-76 (2002)), the contents of which are incorporated herein by reference.

Covalent modifications of an antibody are also included within the scope of this technology. For example, modifications may be made by chemical synthesis or by enzymatic or chemical cleavage of an antibody. Other types of covalent modifications of an antibody are introduced into the molecule by reacting targeted amino acid residues of the antibody with an organic derivatizing agent that is capable of reacting with selected side chains or the N- or C-terminal residues. Example covalent modifications of polypeptides are described in U.S. Pat. No. 5,534,615, specifically incorporated herein by reference. A preferred type of covalent modification of the antibody comprises linking the antibody to one of a variety of non-proteinaceous polymers, e.g., polyethylene glycol, polypropylene glycol, or polyoxyalkylenes, in the manner set forth in, e.g., U.S. Pat. Nos. 4,640,835; 4,496,689; 4,301,144; 4,670,417; 4,791,192 or 4,179,337.

### VIII. Nucleic Acids, Vectors, Host Cells, and Recombinant Methods

The disclosure also provides isolated nucleic acids encoding an antibody, vectors and host cells comprising the nucleic acid, and recombinant techniques for the production of the antibody. A nucleic acid herein may include one or more subsequences, each referred to as a polynucleotide.

Provided herein are nucleic acids (e.g., isolated nucleic acids) comprising a nucleotide sequence that encodes an antibody, or fragment thereof. In some embodiments, a nucleic acid encodes an immunoglobulin heavy chain variable domain of an antibody provided herein. In some embodiments, a nucleic acid encodes an immunoglobulin light chain variable domain of an antibody provided herein. In some embodiments, a nucleic acid encodes an immunoglobulin heavy chain variable domain and an immunoglobulin light chain variable domain of an antibody provided herein. In some embodiments, a nucleic acid comprises a nucleotide sequence that encodes an amino acid sequence of any one of SEQ ID NOS: 43-50 or 51-58. In some of any embodiments, the immunoglobulin heavy chain variable domain comprises the sequence of nucleotides set forth in any of SEQ ID NOs: 43-50. In some of any embodiments, the immunoglobulin heavy chain variable domain comprises the sequence of nucleotides set forth in SEQ ID NO: 43. In some of any embodiments, the immunoglobulin heavy chain variable domain comprises the sequence of nucleotides set forth in SEQ ID NO: 44. In some of any embodiments, the immunoglobulin heavy chain variable domain comprises the sequence of nucleotides set forth in SEQ ID NO: 45. In some of any embodiments, the immunoglobulin heavy chain variable domain comprises the sequence of nucleotides set forth in SEQ ID NO: 46. In some of any embodiments, the immunoglobulin heavy chain variable domain comprises the sequence of nucleotides set forth in SEQ ID NO: 47. In some of any embodiments, the immunoglobulin heavy chain variable domain comprises the sequence of nucleotides set forth in SEQ ID NO: 48. In some of any embodiments, the immunoglobulin heavy chain variable domain comprises the sequence of nucleotides set forth in SEQ ID NO: 49. In some of any embodiments, the immunoglobulin heavy chain variable domain comprises the sequence of nucleotides set forth in SEQ ID NO: 50.

In some of any embodiments, the immunoglobulin light chain variable domain comprises the sequence of nucleotides set forth in any of SEQ ID NOs: 51-58. In some of any embodiments, the immunoglobulin light chain variable domain comprises the sequence of nucleotides set forth in SEQ ID NO: 51. In some of any embodiments, the immunoglobulin light chain variable domain comprises the sequence of nucleotides set forth in SEQ ID NO: 52. In some of any embodiments, the immunoglobulin light chain variable domain comprises the sequence of nucleotides set forth in SEQ ID NO: 53. In some of any embodiments, the immunoglobulin light chain variable domain comprises the sequence of nucleotides set forth in SEQ ID NO: 54. In some of any embodiments, the immunoglobulin light chain variable domain comprises the sequence of nucleotides set forth in SEQ ID NO: 55. In some of any embodiments, the immunoglobulin light chain variable domain comprises the sequence of nucleotides set forth in SEQ ID NO: 56.

In some of any embodiments, the immunoglobulin light chain variable domain comprises the sequence of nucleotides set forth in SEQ ID NO: 57. In some of any embodiments, the immunoglobulin light chain variable domain comprises the sequence of nucleotides set forth in SEQ ID NO: 58.

Provided herein is a nucleotide sequence that encodes the immunoglobulin heavy chain variable domain and the immunoglobulin light chain variable domain of the antibody or antigen binding fragment thereof of any of any of the antibodies or antigen binding fragments provided herein.

In some of any embodiments, the nucleotide sequence that encodes the immunoglobulin heavy chain variable domain comprises the sequence set forth in SEQ ID NO: 43; and the immunoglobulin light chain variable domain comprises the sequence of amino acids set forth in SEQ ID NO: 51. In some of any embodiments, the nucleotide sequence that encodes the immunoglobulin heavy chain variable domain comprises the sequence set forth in SEQ ID NO: 44; and the immunoglobulin light chain variable domain comprises the sequence of amino acids set forth in SEQ ID NO: 52. In some of any embodiments, the nucleotide sequence that encodes the immunoglobulin heavy chain variable domain comprises the sequence set forth in SEQ ID NO: 45; and the immunoglobulin light chain variable domain comprises the sequence of amino acids set forth in SEQ ID NO: 53. In some of any embodiments, the nucleotide sequence that encodes the immunoglobulin heavy chain variable domain comprises the sequence set forth in SEQ ID NO: 46; and the immunoglobulin light chain variable domain comprises the sequence of amino acids set forth in SEQ ID NO: 54. In some of any embodiments, the nucleotide sequence that encodes the immunoglobulin heavy chain variable domain comprises the sequence set forth in SEQ ID NO: 47; and the immunoglobulin light chain variable domain comprises the sequence of amino acids set forth in SEQ ID NO: 55. In some of any embodiments, the nucleotide sequence that encodes the immunoglobulin heavy chain variable domain comprises the sequence set forth in SEQ ID NO: 48; and the immunoglobulin light chain variable domain comprises the sequence of amino acids set forth in SEQ ID NO: 56. In some of any embodiments, the nucleotide sequence that encodes the immunoglobulin heavy chain variable domain comprises the sequence set forth in SEQ ID NO: 49; and the immunoglobulin light chain variable domain comprises the sequence of amino acids set forth in SEQ ID NO: 57. In some of any embodiments, the nucleotide sequence that encodes the immunoglobulin heavy chain variable domain comprises the sequence set forth in SEQ ID NO:50; and the immunoglobulin light chain variable domain comprises the sequence of amino acids set forth in SEQ ID NO: 58.

In some of any embodiments, the nucleotide sequence that encodes the immunoglobulin heavy chain variable domain comprises the sequence set forth in any of SEQ ID NOs: 43-50; and the immunoglobulin light chain variable domain comprises the sequence of amino acids set forth in any of SEQ ID NOs: 51-58.

In some of any embodiments, the nucleic acid further comprises a signal sequence. In some embodiments, the signal sequence comprises the sequence of amino acids in any of SEQ ID NOs: 59-63 or 64-67.

For recombinant production of an antibody, a nucleic acid encoding the antibody may be isolated and inserted into a replicable vector for further cloning (amplification of the DNA) or for expression. In certain instances, an antibody may be produced by homologous recombination. DNA encoding an antibody can be readily isolated and sequenced using conventional procedures (e.g., by using oligonucleotide probes that are capable of binding specifically to genes encoding the heavy and light chains of the antibody). Many vectors are available. The vector components generally include, but are not limited to, one or more of the following: a signal sequence, and origin of replication, one or more marker genes, an enhancer element, a promoter, and a transcription termination sequence.

Suitable host cells for cloning or expressing DNA in vectors herein can be prokaryote, yeast, or higher eukaryote cells. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis, Pseudomonas such as P. aeruginosa, and Streptomyces. One preferred E. coli cloning host is E. coli 294 (ATCC 31,446), although other strains such as E. coli B, E. coli X1776 (ATCC 31,537), and E. coli W3110 (ATCC 27,325) can also be suitable. These examples are illustrative rather than limiting.

In addition to prokaryotes, eukaryotic microbes such as filamentous fungi or yeast are suitable cloning or expression hosts for antibody-encoding vectors. Saccharomyces cerevisiae, or common baker's yeast, is the most commonly used among lower eukaryotic host microorganisms. A number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of antibodies can also be derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori (silk moth) have been identified. A variety of viral strains for transfection are publicly available, e.g., the L-1 variant of Autographa californica NPV and the Bm-5 strain of Bombyx mori NPV, and such viruses may be used as the virus herein according to the present technology, particularly for transfection of Spodoptera frugiperda cells. Plant cell cultures of cotton, corn, potato, soybean, petunia, tomato, and tobacco can also be utilized as hosts.

Suitable host cells for the expression of antibodies also may include vertebrate cells (e.g., mammalian cells). Vertebrate cells may be propagated in culture (tissue culture). Examples of useful mammalian host cell lines include monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse Sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2).

Host cells may be transformed with the above-described expression or cloning vectors for antibody production and cultured in conventional nutrient media modified as appropriate for inducing promoters, selecting transformants, or amplifying the genes encoding the desired sequences. Host cells used to produce antibodies provided herein may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. No. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

When using recombinant techniques, antibodies can be produced intracellularly, in the periplasmic space, or directly secreted into the medium. If the antibody is produced intracellularly, as a first step, the particulate debris, either host cells or lysed fragments, is removed, for example, by centrifugation or ultrafiltration. Carter et al., Bio/Technology 10:163-167 (1992) describe a procedure for isolating antibodies that are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The antibody composition prepared from the cells can be purified using, for example, hydroxylapatite chromatography, gel electrophoresis, dialysis, and affinity chromatography, with affinity chromatography being the preferred purification technique. The suitability of protein A as an affinity ligand depends on the species and isotype of any immunoglobulin Fc domain that is present in the antibody. Protein A can be used to purify antibodies that are based on human heavy chains (Lindmark et al., J. Immunol. Meth. 62:1-13 (1983)). Protein G is recommended for all mouse isotypes and for human γ3 (Guss et al., EMBO J. 5:15671575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a CH3 domain, Bakerbond ABX™ resin (J. T. Baker, Phillipsburg, N.J.) is useful for purification. Other techniques for protein purification, such as fractionation on an ion-exchange column, ethanol precipitation, Reverse Phase HPLC, chromatography on silica, chromatography on heparin SEPHAROSE™, chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between, e.g., about 2.5-4.5, and may be performed at low salt concentrations (e.g., from about 0-0.2 5M salt).

### IX. Pharmaceutical Formulations, Dosing, and Routes of Administration

The present disclosure provides antibodies and related compositions, which may be useful for elimination of CD28-expressing pathogens from the body, for example, and for identification and quantification of the number of expressing pathogens in biological samples, for example.

In some embodiments, any of the antibodies or antigen binding fragments thereof may be formulated in a pharmaceutical composition that is useful for a variety of purposes, including the treatment of diseases or disorders. Pharmaceutical compositions comprising one or more antibodies may be administered using a pharmaceutical device to a patient in need thereof, and according to one embodiment of the technology, kits are provided that include such devices. Such devices and kits may be designed for routine administration, including self-administration, of the pharmaceutical compositions herein.

Therapeutic formulations of an antibody may be prepared for storage by mixing the agent or antibody having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™, or polyethylene glycol (PEG).

In some embodiments, the disease or disorder is associated with CD28 expression. In some embodiments, the disease or disorder is associated with aberrant CD28 expression. In some embodiments, the disease or disorder is associated with Natural Killer (NK), alpha beta T cells, gamma delta T cells, CD8+ T cells, monocytes, or dendritic cells. In some embodiments, the disease or disorder is associated with Natural Killer (NK) cells. In some embodiments, the disease or disorder is associated with alpha beta T cells. In some embodiments, the disease or disorder is associated with gamma delta T cells. In some embodiments, the disease or disorder is associated with CD8+ T cells. In some embodiments, the disease or disorder is associated with monocytes. In some embodiments, the disease or disorder is associated with dendritic cells.

In some embodiments, the disease or disorder is a cancer, an infectious disease, or an autoimmune disorder.

In some embodiments, the disease or disorder is a cancer. In some embodiments, the cancer is metastatic melanoma, a solid tumor, bladder cancer, head and neck squamous cell carcinoma, hepatocellular carcinoma, hepatic metastasis of colonic origin, papillary thyroid carcinoma, acute myeloid leukemia, or asymptomatic myeloma.

In some embodiments, the disease or disorder is an infectious disease. In some embodiments, the infectious disease is human immunodeficiency virus (HIV), chronic hepatitis C, cytomegalovirus, or hantavirus.

In some embodiments, the disease or disorder is an autoimmune disorder. In some embodiments, the autoimmune disorder is Crohn's disease, multiple sclerosis, systemic sclerosis, ocular myasthenia gravis, psoriasis or rheumatoid arthritis.

Formulations herein may also contain more than one active compound as necessary for the particular indication being treated, preferably those with complementary activities that do not adversely affect each other. Such molecules are suitably present in combination in amounts that are effective for the purpose intended.

Formulations for in vivo administration generally are sterile. This may be accomplished for instance by filtration through sterile filtration membranes, for example.

Sustained-release preparations may be prepared. Suitable examples of sustained-release preparations include semipermeable matrices of solid hydrophobic polymers containing the agent/antibody, which matrices are in the form of shaped articles, e.g., films, or microcapsule. Examples of sustained-release matrices include polyesters, hydrogels (for example, poly(2-hydroxyethyl-methacrylate), or poly(vinyl alcohol)), polylactides (U.S. Pat. No. 3,773,919), copolymers of L-glutamic acid and gamma ethyl-L-glutamate, non-degradable ethylene-vinyl acetate, degradable lactic acid-glycolic acid copolymers such as the Lupron Depot® (injectable microspheres composed of lactic acid-glycolic acid copolymer and leuprolide acetate), and poly-D-(−)-3-hydroxybutyric acid. While polymers such as such as ethylene-vinyl acetate and lactic acid-glycolic acid enable release of molecules for over 100 days, certain hydrogels release proteins for shorter time periods. When encapsulated agents/antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thiol-disulfide interchange, stabilization may be achieved by modifying sulfhydryl residues, lyophilizing from acidic solutions, controlling moisture content, using appropriate additives, and developing specific polymer matrix compositions.

For therapeutic applications, antibodies provided herein are administered to a mammal, e.g., a human, in a pharmaceutically acceptable dosage form such as those discussed above, including those that may be administered to a human intravenously as a bolus or by continuous infusion over a period of time, or by intramuscular, intraperitoneal, intra-cerebrospinal, subcutaneous, intra-articular, intrasynovial, intrathecal, oral, topical, or inhalation routes. For the prevention or treatment of disease, the appropriate dosage of agent or antibody will depend on the type of disease to be treated, as defined above, the severity and course of the disease, whether the antibody is administered for preventative or therapeutic purposes, previous therapy, the patient's clinical history and response to the antibody, and the discretion of the attending physician. The antibody is suitably administered to the patient at one time or over a series of treatments.

Depending on the type and severity of the disease, about 1 μg/kg to about 50 mg/kg (e.g., 0.1-20 mg/kg) of antibody may be an initial candidate dosage for administration to the patient, whether, for example, by one or more separate administrations, or by continuous infusion. A typical daily or weekly dosage might range from about 1 μg/kg to about 20 mg/kg or more, depending on the factors mentioned above. For repeated administrations over several days or longer, depending on the condition, the treatment is repeated until a desired suppression of disease symptoms occurs. However, other dosage regimens may be useful. The progress of this therapy is easily monitored by conventional techniques and assays, including, for example, radiographic imaging. Detection methods using the antibody to determine CD28 levels in bodily fluids or tissues may be used in order to optimize patient exposure to the therapeutic antibody.

In some embodiments, a composition comprising an antibody herein can be administered as a monotherapy, and in some embodiments, the composition comprising the antibody can be administered as part of a combination therapy. In some cases, the effectiveness of the antibody in preventing or treating diseases may be improved by administering the antibody serially or in combination with another drug that is effective for those purposes, such as a chemotherapeutic drug for treatment of cancer or a microbial infection. In other cases, the antibody may serve to enhance or sensitize cells to chemotherapeutic treatment, thus permitting efficacy at lower doses and with lower toxicity. Certain combination therapies include, in addition to administration of the composition comprising an antibody that reduces the number of expressing cells, delivering a second therapeutic regimen selected from the group consisting of a chemotherapeutic agent, radiation therapy, surgery, and a combination of any of the foregoing. Such other agents may be present in the composition being administered or may be administered separately. Also, the antibody may be suitably administered serially or in combination with the other agent or modality, e.g., chemotherapeutic drug or radiation for treatment of cancer, infection, and the like, or an immunosuppressive drug.

### X. Research and Diagnostic

Also provided herein are diagnostic reagents comprising an antibody described herein. For example, antibodies provided herein may be used to detect and/or purify CD28 from bodily fluid(s) or tissues. Also provided herein are methods for detecting CD28. For example, a method may comprise contacting a sample (e.g., a biological sample known or suspected to contain) with an antibody provided herein, and, if the sample contains CD28, detecting CD28: antibody complexes. Also provided herein are reagents comprising an antibody described herein and methods for detecting for research purposes.

In some embodiments, any of the antibodies or antigen binding fragments thereof are capable of stimulating T cells. In some embodiments, any of the antibodies or antigen binding fragments thereof are capable of stimulating T cells in the presence of a co-stimulatory agent. In some embodiments, any of the antibodies or antigen binding fragments thereof are capable of stimulating T cells in the presence of a co-stimulatory anti-CD3 antibody. In some embodiments, any of the antibodies or antigen binding fragments thereof are capable of stimulating T cells in the absence of a co-stimulatory agent. In some embodiments, any of the antibodies or antigen binding fragments thereof are capable of stimulating T cells in the absence of a co-stimulatory anti-CD3 antibody. In some embodiments, any of the antibodies or antigen binding fragments thereof can be used in a method of stimulating T cells. In some embodiments, the method involves the use of a co-stimulatory agent or antibody. In some embodiments, the method involves the use of an anti-CD3 co-stimulatory agent or antibody. In some embodiments, the method does not involve the use of a co-stimulatory agent or antibody. In some embodiments, the method does not involve the use of an anti-CD3 co-stimulatory agent or antibody.

Any of the antibodies or antigen binding fragments disclosed herein can be useful in diagnostic assays for detecting its presence in specific cells, tissues, or bodily fluids. Such diagnostic methods may be useful in diagnosis, e.g., of a hyperproliferative disease or disorder. Thus, clinical diagnostic uses as well as research uses are comprehended herein. In some embodiments, an antibody comprises a detectable marker or label. In some embodiments, an antibody is conjugated to a detectable marker or label. For example, for research and diagnostic applications, an antibody may be labeled with a detectable moiety. Numerous labels are available which are generally grouped into the following categories:
(a) Radioisotopes, such as 35S, 14C, 125I, 3H, and 131I. The antibody can be labeled with the radioisotope using the techniques described in Current Protocols in Immunology, Volumes 1 and 2, Coligen et al., Ed. Wiley-Interscience, New York, N. Y., Pubs. (1991), for example, and radioactivity can be measured using scintillation counting.
(b) Fluorescent labels such as rare earth chelates (europium chelates) or fluorescein and its derivatives, rhodamine and its derivatives, dansyl, Lissamine, phycoerythrin, Texas Red and Brilliant Violet™ are available. The fluorescent labels can be conjugated to the antibody using the techniques disclosed in Current Protocols in Immunology, supra, for example. Fluorescence can be quantified using a flow cytometer, imaging microscope or fluorimeter.
(c) Various enzyme-substrate labels are available. The enzyme generally catalyzes a chemical alteration of the chromogenic substrate that can be measured using various techniques. For example, the enzyme may catalyze a color change in a substrate, which can be measured spectrophotometrically. Alternatively, the enzyme may alter the fluorescence or chemiluminescence of the substrate. Techniques for quantifying a change in fluorescence are described above. The chemiluminescent substrate becomes electronically excited by a chemical reaction and may then emit light that can be measured (using a chemilluminometer, for example) or donates energy to a fluorescent acceptor. Examples of enzymatic labels include luciferases (e.g., firefly luciferase and bacterial luciferase), luciferin, 2,3-dihydrophthalazinediones, malate dehydrogenase, urease, peroxidase such as horseradish peroxidase (HRPO), alkaline phosphatase, beta-galactosidase, glucoamylase, lysozyme, saccharide oxidases (e.g., glucose oxidase, galactose oxidase, and glucose-6-phosphate dehydrogenase), heterocyclicoxidases (such as uricase and xanthine oxidase), lactoperoxidase, microperoxidase, and the like. Techniques for conjugating enzymes to antibodies are described in O'Sullivan et al., Methods for the Preparation of Enzyme-Antibody Conjugates for use in Enzyme Immunoassay, in Methods in Enzym. (ed J. Langone & H. Van Vunakis), Academic press, New York, 73:147-166 (1981).

Examples of enzyme-substrate combinations include, for example:
(i) Horseradish peroxidase (HRP) with hydrogen peroxidase as a substrate, where the hydrogen peroxidase oxidizes a dye precursor (e.g., orthophenylene diamine (OPD) or 3,3′,5,5′-tetramethyl benzidine hydrochloride (TMB));
(ii) alkaline phosphatase (AP) with para-Nitrophenyl phosphate as chromogenic substrate; and
(iii) β-D-galactosidase (β-D-Gal) with a chromogenic substrate (e.g., p-nitrophenyl-β-D-galactosidase) or fluorogenic substrate 4-methylumbelliferyl-β-D-galactosidase.

In certain instances, the label is indirectly conjugated with the agent or antibody. The skilled artisan will be aware of various techniques for achieving this. For example, an antibody can be conjugated with biotin and any of the three broad categories of labels mentioned above can be conjugated with avidin, or vice versa. Biotin binds selectively to avidin and thus, the label can be conjugated with the antibody in this indirect manner. Alternatively, to achieve indirect conjugation of the label with the antibody, the antibody is conjugated with a small hapten (e.g., digoxin) and one of the different types of labels mentioned above is conjugated with an anti-hapten antibody (e.g., anti-digoxin antibody). Thus, indirect conjugation of the label with the antibody can be achieved.

In some embodiments, and antibody or antigen binding fragments thereof need not be labeled, and the presence thereof can be detected, e.g., using a labeled antibody which binds to an antibody.

In some embodiments, an antibody herein is immobilized on a solid support or substrate. In some embodiments, an antibody herein is non-diffusively immobilized on a solid support (e.g., the antibody does not detach from the solid support). A solid support or substrate can be any physically separable solid to which an antibody can be directly or indirectly attached including, but not limited to, surfaces provided by microarrays and wells, and particles such as beads (e.g., paramagnetic beads, magnetic beads, microbeads, nanobeads), microparticles, and nanoparticles. Solid supports also can include, for example, chips, columns, optical fibers, wipes, filters (e.g., flat surface filters), one or more capillaries, glass and modified or functionalized glass (e.g., controlled-pore glass (CPG)), quartz, mica, diazotized membranes (paper or nylon), polyformaldehyde, cellulose, cellulose acetate, paper, ceramics, metals, metalloids, semiconductive materials, quantum dots, coated beads or particles, other chromatographic materials, magnetic particles; plastics (including acrylics, polystyrene, copolymers of styrene or other materials, polybutylene, polyurethanes, TEFLON™, polyethylene, polypropylene, polyamide, polyester, polyvinylidenedifluoride (PVDF), and the like), polysaccharides, nylon or nitrocellulose, resins, silica or silica-based materials including silicon, silica gel, and modified silicon, Sephadex®, Sepharose®, carbon, metals (e.g., steel, gold, silver, aluminum, silicon and copper), inorganic glasses, conducting polymers (including polymers such as polypyrole and polyindole); micro or nanostructured surfaces such as nucleic acid tiling arrays, nanotube, nanowire, or nanoparticulate decorated surfaces; or porous surfaces or gels such as methacrylates, acrylamides, sugar polymers, cellulose, silicates, or other fibrous or stranded polymers. In some embodiments, the solid support or substrate may be coated using passive or chemically-derivatized coatings with any number of materials, including polymers, such as dextrans, acrylamides, gelatins or agarose. Beads and/or particles may be free or in connection with one another (e.g., sintered). In some embodiments, a solid support or substrate can be a collection of particles. In some embodiments, the particles can comprise silica, and the silica may comprise silica dioxide. In some embodiments the silica can be porous, and in certain embodiments the silica can be non-porous. In some embodiments, the particles further comprise an agent that confers a paramagnetic property to the particles. In certain embodiments, the agent comprises a metal, and in certain embodiments the agent is a metal oxide, (e.g., iron or iron oxides, where the iron oxide contains a mixture of Fe2+ and Fe3+). An antibody may be linked to a solid support by covalent bonds or by non-covalent interactions and may be linked to a solid support directly or indirectly (e.g., via an intermediary agent such as a spacer molecule or biotin).

Antibodies and antigen binding fragments thereof provided herein may be employed in any known assay method, such as flow cytometry, immunohistochemistry, immunofluorescence, mass cytometry (e.g., Cytof instrument), competitive binding assays, direct and indirect sandwich assays, and immunoprecipitation assays. Zola, Monoclonal Antibodies: A Manual of Techniques, pp. 147-158 (CRC Press, Inc. 1987). Flow cytometry and mass cytometry assays generally involve the use of a single primary antibody to specifically identify the presence of the target molecule expressed on the surface of a dispersed suspension of individual cells. The dispersed cells are typically obtained from a biological fluid sample, e.g., blood, but may also be obtained from a dispersion of single cells prepared from a solid tissue sample such as spleen or tumor biopsy. The primary antibody may be directly conjugated with a detectable moiety, e.g., a fluorophore such as phycoerythrin for flow cytometry or a heavy metal chelate for mass cytometry. Alternatively, the primary antibody may be unlabeled or labeled with an undetectable tag such as biotin, and the primary antibody is then detected by a detectably labeled secondary antibody that specifically recognizes the primary antibody itself or the tag on the primary antibody. The labeled cells are then analyzed in an instrument capable of single cell detection, e.g., flow cytometer, mass cytometer, fluorescence microscope or brightfield light microscope, to identify those individual cells in the dispersed population or tissue sample that express the target recognized by the primary antibody. Detailed description of the technological basis and practical application of flow cytometry principles may be found in, e.g., Shapiro, Practical Flow Cytometry, 4th Edition, Wiley, 2003.

Sandwich assays involve the use of two antibodies, each capable of binding to a different immunogenic portion, or epitope, of the protein that is detected. In a sandwich assay, the test sample analyte is bound by a first antibody that is immobilized on a solid support, and thereafter a second antibody binds to the analyte, thus forming an insoluble three-part complex. See, e.g., U.S. Pat. No. 4,376,110. The second antibody may itself be labeled with a detectable moiety (direct sandwich assays) or may be measured using an anti-immunoglobulin antibody that is labeled with a detectable moiety (indirect sandwich assay). For example, one type of sandwich assay is an ELISA assay, in which case the detectable moiety is an enzyme. In a cell ELISA, the target cell population may be attached to the solid support using antibodies first attached to the support and that recognize different cell surface proteins. These first antibodies capture the cells to the support. CD28 on the surface of the cells can then be detected by adding any of the anti-CD28 antibodies or antigen-binding fragments thereof described herein to the captured cells and detecting the amount of the anti-CD28 antibody or antigen binding fragment thereof attached to the cells. In certain instances, fixed and permeabilized cells may be used, and in such instances, both surface CD28 and intracellular CD28 may be detected.

In some embodiments, any of the antibodies or antigen binding fragments thereof provided herein are formulated for immunohistochemical analysis. In some embodiments, immunohistochemical analysis includes the use of samples. In some embodiments, immunohistochemical analysis includes the use of blood and/or tissue samples. In some embodiments, the sample may be fresh or frozen or may be embedded in paraffin and fixed with a preservative such as formalin. In some embodiments, the sample is a formalin-fixed paraffin-embedded (FFPE) sample. In some embodiments, the FFPE sample is saturated with formalin (i.e. formaldehyde) and then embedded in a block of paraffin wax. In some embodiments, the FFPE sample is stable at room temperature. In some embodiments, all of the structures in the FFPE sample are preserved. In some embodiments, the intracellular and surface proteins in the FFPE sample are preserved. In some embodiments, the mRNA in the FFPE sample is preserved. In some embodiments, the mRNA, intracellular and surface proteins in the FFPE sample are preserved. In some embodiments, the surface proteins in the FFPE sample are denatured.

In some embodiments, any of the anti-CD28 antibodies or antigen binding fragments thereof provided herein are capable of detecting CD28 in a formalin-fixed paraffin-embedded sample. In some embodiments, any of the anti-CD28 antibodies or antigen binding fragments thereof provided herein are capable of detecting CD28 on the surface of a in a formalin-fixed paraffin-embedded sample. In some embodiments, any of the anti-CD28 antibodies or antigen binding fragments thereof provided herein are capable of detecting intracellular CD28 in a formalin-fixed paraffin-embedded sample. In some embodiments, any of the anti-CD28 antibodies or antigen binding fragments thereof provided herein are capable of detecting intracellular CD28, and CD28 on the surface of a formalin-fixed paraffin-embedded sample.

In some embodiments, the sample is a fresh sample that has been frozen. In some embodiments, the sample is a fresh sample that has been cryogenically frozen. In some embodiments, the sample is flash frozen. In some embodiments, the sample if flash frozen and stored at 80° C. In some embodiments, all of the structures in the flash frozen sample are preserved. In some embodiments, the intracellular and surface proteins in the flash frozen sample are preserved. In some embodiments, the mRNA in the flash frozen sample is preserved. In some embodiments, the mRNA, intracellular and surface proteins in the flash frozen sample are preserved. In some embodiments, the surface proteins in the flash frozen sample are denatured.

In some embodiments, any of the antibodies or antigen binding fragments thereof provided herein are capable of detecting CD28 in a frozen sample. In some embodiments, any of the antibodies or antigen binding fragments thereof provided herein are capable of detecting CD28 on the surface of a frozen sample. In some embodiments, any of the antibodies or antigen binding fragments thereof provided herein are capable of detecting intracellular CD28 in a frozen sample. In some embodiments, any of the antibodies or antigen binding fragments thereof provided herein are capable of detecting intracellular CD28, and CD28 on the surface of a frozen sample.

The antibodies herein also may be used for in vivo diagnostic assays. Generally, the antibody is labeled with a radionuclide (such as 111 In, 99Tc, 14C, 131I, 125I, 3H, 32P, or 35S) so that the bound target molecule can be localized using immunoscintillography.

### XI. Detection of CD28

Provided herein are antibodies and methods for detecting CD28. In some embodiments, antibodies and methods are provided for detecting CD28 in a biological sample. In some embodiments, the CD28 is detected on the surface of the cell. In some embodiments, the CD28 is detected intracellularly. In some embodiments, the detection of CD28 is in vitro. In some embodiments, the detection of CD28 is in vivo.

In some embodiments, the biological sample is a solid tissue, fluid, or cell. The solid tissue may comprise solid tissue from one or more of adipose tissue, bladder, bone, brain breast cervix, endothelium, gallbladder, kidney, liver, lung, lymph, ovary, prostate, salivary gland, stomach, testis, thyroid, urethra, uterus, vagina, and vulva. In some embodiments, the fluid comprises one or more of amniotic fluid, bile, blood, breast milk, breast fluid, cerebrospinal fluid, lavage fluid, lymphatic fluid, mucous, plasma, saliva, semen, serum, spinal fluid, sputum, tears, umbilical cord blood, urine, and vaginal fluid.

In some embodiments, the sample comprises immune cells. In some embodiments, the sample comprises a heterogeneous population of immune cells. In some embodiments, the immune cell is selected from B cells, plasmacytoid dendritic cells (pDCs), lymphocytes, leukocytes, T cells, monocytes, macrophages, neutrophils, myeloid dendritic cells (mDCs), innate lymphoid cells, mast cells, eosinophils, basophils, natural killer cells, and peripheral blood mononuclear cells (PBMCs).

In some of any embodiments, any of the antibodies or antigen binding fragments thereof provided herein can be used in the characterization of single cells by measurement of gene-expression levels and cellular proteins. Among such known single cell sequencing platforms suitable for integration with the antibodies or antigen binding fragments thereof described herein is the Drop-seq method, including, but not limited to, microfluidic, plate-based, or microwell, Seq-Well™ method and adaptations of the basic protocol, and InDrop™ method. In another embodiment, a single cell sequencing platform suitable for integration with the antibodies or antigen binding fragments thereof described herein is 10× genomics single cell 3′ solution or single cell V (D) J solution, either run on Chromium controller, or dedicated Chromium single cell controller. Other suitable sequencing methods include Wafergen iCell8™ method, Microwell-seq method, Fluidigm CI™ method and equivalent single cell products. Still other known sequencing protocols useful with the antibodies or antigen binding fragments thereof described herein include BD Resolve™ single cell analysis platform and ddSeq (from Illumina® Bio-Rad® SureCell™ WTA 3′ Library Prep Kit for the ddSEQ™ System, 2017, Pub. No. 1070-2016-014-B, Illumina Inc., Bio-Rad Laboratories, Inc.). In still other embodiment, the antibodies or antigen binding fragments thereof described herein are useful with combinatorial indexing based approaches (sci-RNA-seq™ method or SPLIT-seq™ method) and Spatial Transcriptomics, or comparable spatially resolved sequencing approaches. The methods and compositions described herein can also be used as an added layer of information on standard index sorting (FACS) and mRNA-sequencing-based approaches.

In some of any embodiments, any of the antibodies or antigen binding fragments thereof described herein can be used to detect the presence, absence or amount of the various nucleic acids, proteins, targets, oligonucleotides, amplification products and barcodes described herein.

In some embodiments, the biological sample is from a healthy subject. In some embodiments, the sample is from a subject with a disease or condition. In some embodiments, the detection of CD28 indicates the presence or absence of a disease or disorder. In some embodiments, the disease or disorder is a cancer, an autoimmune disorder, an inflammatory disorder, a neurologic disorder, or an infection. In some embodiments, the cancer is the cancer is acute myeloid leukemia, acute lymphoblastic leukemia, colorectal, ovarian, gynecologic, liver, glioblastoma, Hodgkin lymphoma, chronic lymphocytic leukemia, esophagus, gastric, pancreas, colon, kidney, head and neck, lung and melanoma.

In some embodiments, the disease or disorder is associated with CD28 expression, In some embodiments, the disease or disorder is associated with aberrant CD28 expression. In some embodiments, the disease or disorder is associated with Natural Killer (NK), alpha beta T cells, gamma delta T cells, CD8+ T cells, monocytes, or dendritic cells. In some embodiments, the disease or disorder is associated with Natural Killer (NK) cells. In some embodiments, the disease or disorder is associated with alpha beta T cells. In some embodiments, the disease or disorder is associated with gamma delta T cells. In some embodiments, the disease or disorder is associated with CD8+ T cells. In some embodiments, the disease or disorder is associated with monocytes. In some embodiments, the disease or disorder is associated with dendritic cells. In some of any embodiments, the disease or disorder is chosen from non-viral cancers, virus-associated cancers, cancers associated with HBV infection, cancers associated with Epstein-Barr virus (EBV) infection, cancers associated with polyomavirus infection, erythema nodosum leprosum (ENL), autoimmune diseases, autoimmune inflammation, autoimmune thyroid diseases, B-cell lymphoma, T-cell lymphoma, acute myeloid leukemia, Hodgkin's Disease, acute myelogenous leukemia, acute myelomonocytic leukemia, acute lymphoblastic leukemia, chronic myelogenous leukemia, chronic lymphocytic leukemia, B cell large cell lymphoma, malignant lymphoma, acute leukemia, lymphosarcoma cell leukemia, B-cell leukemias, myelodysplastic syndromes, solid phase cancer, herpes viral infections, and/or rejection of transplanted tissues or organs.

In some embodiments, the disease or disorder is a cancer. In some embodiments, the cancer is metastatic melanoma, a solid tumor, bladder cancer, head and neck squamous cell carcinoma, hepatocellular carcinoma, hepatic metastasis of colonic origin, papillary thyroid carcinoma, acute myeloid leukemia, or asymptomatic myeloma.

In some embodiments, the disease or disorder is an infectious disease. In some embodiments, the infectious disease is human immunodeficiency virus (HIV), chronic hepatitis C, cytomegalovirus, or hantavirus.

In some embodiments, the disease or disorder is an autoimmune disorder. In some embodiments, the autoimmune disorder is Crohn's disease, multiple sclerosis, systemic sclerosis, ocular myasthenia gravis, psoriasis or rheumatoid arthritis.

In some embodiments, the antibody or antigen binding fragment thereof binds to a CD28 expressing cell, and activates a T cell. In some embodiments, the T cell activation comprises a first signal induced via a TCR and a second signal induced by binding of the CD28 to B7-1 (also known as CD80) or B7-2 (also known as CD86). In some embodiments, the T cell activation does not comprise a first signal induced via a TCR. In some embodiments, the antibody or antigen binding fragment thereof bound to the CD28 expressing cell enhances the production of cytokines. In some embodiments, the antibody or antigen binding fragment thereof bound to the CD28 expressing cell initiates differentiation of the cell into a memory phenotype.

In some embodiments, any of the antibodies or antigen binding fragments thereof can be used in generating a nucleic acid molecule comprising all or a portion of the sequence of the oligonucleotide or a complement thereof. In some of any embodiments, the antibody or antigen binding fragment thereof can be used in a method of associating presence or abundance of CD28 with a location of interest of a tissue sample.

In some embodiments, any of the antibodies or antigen binding fragments thereof can be used in the construction of a protein library. In some of any embodiments, the construction of a protein library comprises sequencing. In some of any embodiments, the construction of a protein library comprises the use of flow cytometry.

In some of any embodiments, provided herein is a method of detecting CD28, comprising: a) contacting a sample with the antibody or antigen binding fragment thereof of any of the antibodies or antigen binding fragments thereof under conditions to bind said antibody or antigen binding fragment thereof to a CD28 receptor on said sample, wherein the binding generates the production of a receptor/antibody or antigen binding fragment thereof of complex; b) detecting the presence of the receptor/antibody or antigen binding fragment thereof of complexes; c) wherein the detecting comprises the presence or absence of the CD28 receptor on said sample.

In some of any embodiments, provided herein is a method of treating or preventing a disease or disorder associated with CD28 in a subject, comprising: a) contacting a sample known or suspected to contain CD28 with the antibody or antigen binding fragment thereof any of the antibodies or antigen binding fragments thereof, b) detecting the presence of complexes comprising CD28 and the antibody or antigen binding fragment thereof; wherein the presence of the complexes indicates the presence of a disease or disorder; and c) administering to the subject the antibody or antigen binding fragment thereof of any of the antibodies or antigen binding fragments thereof

In some of any embodiments, provided herein is a method of diagnosing a disease or disorder, comprising: a) isolating a sample from a subject, b) incubating the sample with the antibody or antigen binding fragment thereof of any of any of the antibodies or antigen binding fragments thereof, for a period of time sufficient to generate CD28: anti-CD28 complexes; c) detecting the presence or absence of the CD28: anti-CD28 complexes from the isolated tissue, and d) associating presence or abundance of CD28 with a location of interest of a tissue sample.

In some of any embodiments, the increase of CD28 over a control level in the location of interest of the tissue sample is indicative of a disease or disorder in a subject.

In some of any embodiments, the detection comprises hybridization of a detectable moiety to the antibody or antigen binding fragment thereof. In some of any embodiments, the sample is contacted with a second antibody. In some of any embodiments, the second antibody is an antibody comprising a detectable moiety. In some of any embodiments, the detectable moiety comprises an oligonucleotide. In some of any embodiments, the detectable moiety comprises a fluorescent label. In some of any embodiments, the measurement comprises sequencing. In some of any embodiments, the detectable moiety comprises immunofluorescence. In some of any embodiments, the sample is a formalin-fixed paraffin-embedded sample. In some of any embodiments, the sample comprises a cell. In some of any embodiments, the sample comprises a tissue sample.

### XII. Kits

In some embodiments, any of the antibodies or antigen binding fragments thereof herein may be provided in a kit, for example, a packaged combination of reagents in predetermined amounts with instructions for use (e.g., instructions for performing a diagnostic assay; instructions for performing a laboratory assay). In some embodiments, the kit is a diagnostic kit configured to detect in a sample (e.g., a biological sample). Where the antibody is labeled with a fluorophore, the kit may include an identical isotype negative control irrelevant antibody to control for non-specific binding of the antibody. Where the antibody is labeled with an enzyme, the kit may include substrates and cofactors required by the enzyme (e.g., substrate precursor which provides the detectable chromophore or fluorophore). Additional additives may be included such as stabilizers, buffers (e.g., a block buffer or lysis buffer), and the like. The relative amounts of the various reagents may be varied widely to provide for concentrations in solution of the reagents that substantially optimize the sensitivity of the assay. In certain instances, reagents may be provided as dry powders (e.g., lyophilized powder), including excipients that on dissolution will provide a reagent solution having the appropriate concentration.

### XIII. Exemplary Embodiments

1. An isolated antibody or antigen binding fragment thereof that binds CD28 or a portion thereof, comprising:
a) an immunoglobulin heavy chain variable domain comprising:
   (i) a heavy chain complementary determining region 1 (CDRH1) comprising the sequence X₁YX₃X₄X₅ (SEQ ID NO: 68), wherein X₁ is S or D; X₃ is T, W, I or N; X₄ is M or I and X₅ is H, N or S;
   (ii) a heavy chain complementary determining region 2 (CDRH2) comprising the sequence X₁X₂X₃X₄X₅X₆GX₈X₉X₁₀X₁₁X₁₂X₁₃X₁₄X₁₅KX₁₇ (SEQ ID NO: 69), wherein X₁ is Y, N, W or I; X₂ is I or F; X₃ is D, Y or S; X₄ is P or N, X₅ is G or Y; X₆ is S, Y or G; X₈ is Y, R, S, A or T; X₉ is S, T or I; X₁₀ is E, I, N, T or Y; X₁₁ is F or Y; X₁₂ is N, D or P; X₁₃ is H, E, Q or D; X₁₄ is K, R or T; X₁₅ is F or V; and X₁₇ is D, S or G; and
   (iii) a heavy chain complementary determining region 3 (CDRH3) comprising the sequence X₁X₂X₃X₄X₅X₆X₇X₈X₉X₁₀DY (SEQ ID NO: 70), wherein X₁ is R, S, F or H; X₂ is Q, H, E, G or R; X₃ is D, Y, For P; X₄ is N, G, R, Y or T; X₅ is S, G or D; X₆ is G, S, or no amino acid; X₇ is Y, F, or no amino acid; X₅ is D or no amino acid; X₉ is S, A or V; and X₁₀ is F, M or L; and
b) an immunoglobulin light chain variable domain comprising:
   (i) a light chain complementary determining region 1 (CDRL1) comprising the sequence X₁X₂SX₄X₅X₆X₇X₈X₉X₁₀X₁₁X₁₂X₁₃X₁₄X₁₅X₁₆ (SEQ ID NO: 71), wherein X₁ is R, H or K; X₂ is T, A or S; X₄ is E or Q; X₅ is N, S or I; X₆ is I, L or V; X₇ is Y, A or G; X₈ is S, V, N or T; X₉ is N, W, S or D; X₁₀ is L, For V; X₁₁ is A, N or G; X₁₂ is N or no amino acid; X₁₃ is T or no amino acid; X₁₄ is Y or no amino acid; X₁₅ is L or no amino acid; and X₁₆ is S or no amino acid;
   (ii) a light chain complementary determining region 2 (CDRL2) comprising the sequence X₁X₂X₃X₄X₅X₆X₇ (SEQ ID NO: 72), wherein X₁ is A, K, G or S; X₂ is A or I; X₃ is T or S; X₄ is N or K; X₅ is L or R; X₆ is A, H, For Y; and X₇ is D, T or S; and
   (iii) a light chain complementary determining region 3 (CDRL3) comprising the sequence X₁X₂X₃X₄X₅X₆PX₈T (SEQ ID NO: 73), wherein X₁ is Q, L or H; X₂ is H or Q; X₃ is F, G or Y; X₄ is W, Q, T or S; X₅ is G, S, H or R; X₆ is I, Y, Tor Q; and X₈ is W, For Y.

2. The antibody or antigen binding fragment thereof of embodiment 1, wherein the immunoglobulin heavy chain variable domain comprises: a CDRH1 comprising the sequence of amino acids set forth in SEQ ID NO: 13, 14, 15, 16 or 17, or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 13, 14, 15, 16 or 17; a CDRH2 comprising the sequence of amino acids set forth in SEQ ID NO: 18, 19, 20, 21 or 22, or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 18, 19, 20, 21 or 22; and a CDRH3 comprising the sequence of amino acids set forth in SEQ ID NO: 23, 24, 25, 26 or 27, or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 23, 24, 25, 26 or 27.

3. The antibody or antigen binding fragment thereof of embodiment 1 or embodiment 2, wherein the immunoglobulin heavy chain variable domain comprises: a CDRH1 comprising the sequence of amino acids set forth in SEQ ID NO: 13, 14, 15, 16 or 17; a CDRH2 comprising the sequence of amino acids set forth in SEQ ID NO: 18, 19, 20, 21 or 22; and a CDRH3 comprising the sequence of amino acids set forth in SEQ ID NO: 23, 24, 25, 26 or 27.

4. The antibody or antigen binding fragment thereof of any of embodiments 1-3, wherein the immunoglobulin light chain variable domain comprises: a CDRL1 comprising the sequence of amino acids set forth in SEQ ID NO: 28, 29, 30, 31 or 32, or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 28, 29, 30, 31 or 32; a CDRL2 comprising the sequence of amino acids set forth in SEQ ID NO: 33, 34, 35, 36 or 37, or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 33, 34, 35, 36 or 37; and a CDRL3 comprising the sequence of amino acids set forth in SEQ ID NO: 38, 39, 40, 41 or 42, or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 38, 39, 40, 41 or 42.

5. The antibody or antigen binding fragment thereof of any of embodiments 1-4, wherein the immunoglobulin light chain variable domain comprises: a CDRL1 comprising a sequence of amino acids set forth in SEQ ID NO: 28, 29, 30, 31 or 32; a CDRL2 comprising a sequence of amino acids set forth in SEQ ID NO: 33, 34, 35, 36 or 37; and a CDRL3 comprising a sequence of amino acids set forth in SEQ ID NO: 38, 39, 40, 41 or 42.

6. The antibody or antigen binding fragment thereof of any of embodiments 1-5, wherein the CDRH1 comprises the sequence set forth in SEQ ID NO: 13, or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 13, the CDRH2 comprises the sequence set forth in SEQ ID NO: 18, or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 18; and the CDRH3 comprises the sequence set forth in SEQ ID NO: 23, or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 23.

7. The antibody or antigen binding fragment thereof of any of embodiments 1-6, wherein the CDRH1 comprises the sequence set forth in SEQ ID NO: 13, the CDRH2 comprises the sequence set forth in SEQ ID NO: 18; and the CDRH3 comprises the sequence set forth in SEQ ID NO: 23.

8. The antibody or antigen binding fragment thereof of any of embodiments 1-5, wherein the CDRH1 comprises the sequence set forth in SEQ ID NO: 14, or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 14; the CDRH2 comprises the sequence set forth in SEQ ID NO: 19 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 19; and the CDRH3 comprises the sequence set forth in SEQ ID NO: 24 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 24.

9. The antibody or antigen binding fragment thereof of any of embodiments 1-5 and 8, wherein the CDRH1 comprises the sequence set forth in SEQ ID NO: 14, the CDRH2 comprises the sequence set forth in SEQ ID NO: 19; and the CDRH3 comprises the sequence set forth in SEQ ID NO: 24.

10. The antibody or antigen binding fragment thereof of any of embodiments 1-5, wherein the CDRH1 comprises the sequence set forth in SEQ ID NO: 15, or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 15; the CDRH2 comprises the sequence set forth in SEQ ID NO: 20 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 20; and the CDRH3 comprises the sequence set forth in SEQ ID NO: 25 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:25.

11. The antibody or antigen binding fragment thereof of any of embodiments 1-5 and 10, wherein the CDRH1 comprises the sequence set forth in SEQ ID NO: 15, the CDRH2 comprises the sequence set forth in SEQ ID NO: 20; and the CDRH3 comprises the sequence set forth in SEQ ID NO: 25.

12. The antibody or antigen binding fragment thereof of any of embodiments 1-5, wherein the CDRH1 comprises the sequence set forth in SEQ ID NO: 16, or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 16; the CDRH2 comprises the sequence set forth in SEQ ID NO: 21 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 21; and the CDRH3 comprises the sequence set forth in SEQ ID NO: 26 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:26.

13. The antibody or antigen binding fragment thereof of any of embodiments 1-5 and 12, wherein the CDRH1 comprises the sequence set forth in SEQ ID NO: 16, the CDRH2 comprises the sequence set forth in SEQ ID NO: 21; and the CDRH3 comprises the sequence set forth in SEQ ID NO: 26.

14. The antibody or antigen binding fragment thereof of any of embodiments 1-5, wherein the CDRH1 comprises the sequence set forth in SEQ ID NO: 17, or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 17; the CDRH2 comprises the sequence set forth in SEQ ID NO: 22 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 22; and the CDRH3 comprises the sequence set forth in SEQ ID NO: 27 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:27.

15. The antibody or antigen binding fragment thereof of any of embodiments 1-5 and 14, wherein the CDRH1 comprises the sequence set forth in SEQ ID NO: 17, the CDRH2 comprises the sequence set forth in SEQ ID NO: 22; and the CDRH3 comprises the sequence set forth in SEQ ID NO: 27.

16. The antibody or antigen binding fragment thereof of any of embodiments 1-15, wherein the CDRL1 comprises the sequence set forth in SEQ ID NO: 28, or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 28; the CDRL2 comprises the sequence set forth in SEQ ID NO: 33 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 33; and the CDRL3 comprises the sequence set forth in SEQ ID NO: 38 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 38.

17. The antibody or antigen binding fragment thereof of any of embodiments 1-16, wherein the CDRL1 comprises the sequence set forth in SEQ ID NO: 28, the CDRL2 comprises the sequence set forth in SEQ ID NO: 33; and the CDRL3 comprises the sequence set forth in SEQ ID NO: 38.

18. The antibody or antigen binding fragment thereof of any of embodiments 1-15, wherein the CDRL1 comprises the sequence set forth in SEQ ID NO: 29 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:29, the CDRL2 comprises the sequence set forth in SEQ ID NO: 34 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:34; and the CDRL3 comprises the sequence set forth in SEQ ID NO: 39 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 39.

19. The antibody or antigen binding fragment thereof of any of embodiments 1-15 and 18, wherein the CDRL1 comprises the sequence set forth in SEQ ID NO: 29, the CDRL2 comprises the sequence set forth in SEQ ID NO: 34; and the CDRL3 comprises the sequence set forth in SEQ ID NO: 39.

20. The antibody or antigen binding fragment thereof of any of embodiments 1-15, wherein the CDRL1 comprises the sequence set forth in SEQ ID NO: 30 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:30, the CDRL2 comprises the sequence set forth in SEQ ID NO: 35 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:35; and the CDRL3 comprises the sequence set forth in SEQ ID NO: 40 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 40.

21. The antibody or antigen binding fragment thereof of any of embodiments 1-15 and 20, wherein the CDRL1 comprises the sequence set forth in SEQ ID NO: 30, the CDRL2 comprises the sequence set forth in SEQ ID NO: 35; and the CDRL3 comprises the sequence set forth in SEQ ID NO: 40.

22. The antibody or antigen binding fragment thereof of any of embodiments 1-15, wherein the CDRL1 comprises the sequence set forth in SEQ ID NO: 31 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:31, the CDRL2 comprises the sequence set forth in SEQ ID NO: 36 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:36; and the CDRL3 comprises the sequence set forth in SEQ ID NO: 41 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 41.

23. The antibody or antigen binding fragment thereof of any of embodiments 1-15 and 22, wherein the CDRL1 comprises the sequence set forth in SEQ ID NO: 31, the CDRL2 comprises the sequence set forth in SEQ ID NO: 36; and the CDRL3 comprises the sequence set forth in SEQ ID NO: 41.

24. The antibody or antigen binding fragment thereof of any of embodiments 1-15, wherein the CDRL1 comprises the sequence set forth in SEQ ID NO: 28 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:28, the CDRL2 comprises the sequence set forth in SEQ ID NO: 35 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:35; and the CDRL3 comprises the sequence set forth in SEQ ID NO: 40 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 40.

25. The antibody or antigen binding fragment thereof of any of embodiments 1-15 and 24, wherein the CDRL1 comprises the sequence set forth in SEQ ID NO: 28, the CDRL2 comprises the sequence set forth in SEQ ID NO: 35; and the CDRL3 comprises the sequence set forth in SEQ ID NO: 40.

26. The antibody or antigen binding fragment thereof of any of embodiments 1-15, wherein the CDRL1 comprises the sequence set forth in SEQ ID NO: 32 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:32, the CDRL2 comprises the sequence set forth in SEQ ID NO: 37 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:37; and the CDRL3 comprises the sequence set forth in SEQ ID NO: 42 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 42.

27. The antibody or antigen binding fragment thereof of any of embodiments 1-15 and 26, wherein the CDRL1 comprises the sequence set forth in SEQ ID NO: 32, the CDRL2 comprises the sequence set forth in SEQ ID NO: 37; and the CDRL3 comprises the sequence set forth in SEQ ID NO: 42.

28. The antibody or antigen binding fragment thereof of any of embodiments 1-27, wherein the CDRH1 comprises the sequence of amino acids set forth in SEQ ID NO: 13, 14, 15, 16 or 17, or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 13, 14, 15, 16 or 17; the CDRH2 comprises a sequence of amino acids set forth in SEQ ID NO: 18, 19, 20, 21 or 22, or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 18, 19, 20, 21 or 22; the CDRH3 comprises a sequence of amino acids set forth in SEQ ID NO: 23, 24, 25, 26 or 27, or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:23, 24, 25, 26 or 27; the CDRL1 comprises a sequence of amino acids set forth in SEQ ID NO: 28, 29, 30, 31 or 32, or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 28, 29, 30, 31 or 32; the CDRL2 comprises a sequence of amino acids set forth in SEQ ID NO: 33, 34, 35, 36 or 37, or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 33, 34, 35, 36 or 37; the CDRL3 comprises a sequence of amino acids set forth in SEQ ID NO: 38, 39, 40, 41 or 42, or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 38, 39, 40, 41 or 42.

29. The antibody or antigen binding fragment thereof of any of embodiments 1-28, wherein the CDRH1 comprises the sequence of amino acids set forth in SEQ ID NO: 13, 14, 15, 16 or 17; the CDRH2 comprises the sequence of amino acids set forth in SEQ ID NO: 18, 19, 20, 21 or 22; the CDRH3 comprises the sequence of amino acids set forth in SEQ ID NO: 23, 24, 25, 26 or 27; the CDRL1 comprises the sequence of amino acids set forth in SEQ ID NO: 28, 29, 30, 31 or 32; the CDRL2 comprises the sequence of amino acids set forth in SEQ ID NO: 33, 34, 35, 36 or 37; and the CDRL3 comprises the sequence of amino acids set forth in SEQ ID NO: 38, 39, 40, 41 or 42.

30. The antibody or antigen binding fragment thereof of any of embodiments 1-29, wherein the CDRH1 comprises the sequence set forth in SEQ ID NO: 13 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 13, the CDRH2 comprises the sequence set forth in SEQ ID NO: 18 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:18; the CDRH3 comprises the sequence set forth in SEQ ID NO: 23 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:23; the CDRL1 comprises the sequence set forth in SEQ ID NO: 28 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:28, the CDRL2 comprises the sequence set forth in SEQ ID NO: 33 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:33; and the CDRL3 comprises the sequence set forth in SEQ ID NO: 38 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 38.

31. The antibody or antigen binding fragment thereof of any of embodiments 1-30, wherein the CDRH1 comprises the sequence set forth in SEQ ID NO: 13, the CDRH2 comprises the sequence set forth in SEQ ID NO: 18; the CDRH3 comprises the sequence set forth in SEQ ID NO: 23; the CDRL1 comprises the sequence set forth in SEQ ID NO: 28, the CDRL2 comprises the sequence set forth in SEQ ID NO: 33; and the CDRL3 comprises the sequence set forth in SEQ ID NO: 38.

32. The antibody or antigen binding fragment thereof of any of embodiments 1-29, wherein the CDRH1 comprises the sequence set forth in SEQ ID NO: 14 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 14; the CDRH2 comprises the sequence set forth in SEQ ID NO: 19 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 19; the CDRH3 comprises the sequence set forth in SEQ ID NO: 24 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:24; the CDRL1 comprises the sequence set forth in SEQ ID NO: 29 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:29, the CDRL2 comprises the sequence set forth in SEQ ID NO: 34 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:34; and the CDRL3 comprises the sequence set forth in SEQ ID NO: 39 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 39.

33. The antibody or antigen binding fragment thereof of any of embodiments 1-29 and 32, wherein the CDRH1 comprises the sequence set forth in SEQ ID NO: 14, the CDRH2 comprises the sequence set forth in SEQ ID NO: 119; the CDRH3 comprises the sequence set forth in SEQ ID NO: 24; the CDRL1 comprises the sequence set forth in SEQ ID NO: 29, the CDRL2 comprises the sequence set forth in SEQ ID NO: 34; and the CDRL3 comprises the sequence set forth in SEQ ID NO: 39.

34. The antibody or antigen binding fragment thereof of any of embodiments 1-29, wherein the CDRH1 comprises the sequence set forth in SEQ ID NO: 15 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 15, the CDRH2 comprises the sequence set forth in SEQ ID NO: 20 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:20; the CDRH3 comprises the sequence set forth in SEQ ID NO: 25 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:25; the CDRL1 comprises the sequence set forth in SEQ ID NO: 30 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:30, the CDRL2 comprises the sequence set forth in SEQ ID NO: 35 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:35; and the CDRL3 comprises the sequence set forth in SEQ ID NO: 40 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 40.

35. The antibody or antigen binding fragment thereof of any of embodiments 1-29 and 34, wherein the CDRH1 comprises the sequence set forth in SEQ ID NO: 15, the CDRH2 comprises the sequence set forth in SEQ ID NO: 20; the CDRH3 comprises the sequence set forth in SEQ ID NO: 25; the CDRL1 comprises the sequence set forth in SEQ ID NO: 30, the CDRL2 comprises the sequence set forth in SEQ ID NO: 35; and the CDRL3 comprises the sequence set forth in SEQ ID NO: 40.

36. The antibody or antigen binding fragment thereof of any of embodiments 1-29, wherein the CDRH1 comprises the sequence set forth in SEQ ID NO: 16 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 16, the CDRH2 comprises the sequence set forth in SEQ ID NO: 21 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:21; the CDRH3 comprises the sequence set forth in SEQ ID NO: 26 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:26; the CDRL1 comprises the sequence set forth in SEQ ID NO: 30 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:30, the CDRL2 comprises the sequence set forth in SEQ ID NO: 35 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:35; and the CDRL3 comprises the sequence set forth in SEQ ID NO: 40 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 40.

37. The antibody or antigen binding fragment thereof of any of embodiments 1-29 and 36, wherein the CDRH1 comprises the sequence set forth in SEQ ID NO: 16, the CDRH2 comprises the sequence set forth in SEQ ID NO: 21; the CDRH3 comprises the sequence set forth in SEQ ID NO: 26; the CDRL1 comprises the sequence set forth in SEQ ID NO: 30, the CDRL2 comprises the sequence set forth in SEQ ID NO: 35; and the CDRL3 comprises the sequence set forth in SEQ ID NO: 40.

38. The antibody or antigen binding fragment thereof of any of embodiments 1-29, wherein the CDRH1 comprises the sequence set forth in SEQ ID NO: 16 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 16, the CDRH2 comprises the sequence set forth in SEQ ID NO: 21 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:21; the CDRH3 comprises the sequence set forth in SEQ ID NO: 26 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:26; the CDRL1 comprises the sequence set forth in SEQ ID NO: 31 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:31, the CDRL2 comprises the sequence set forth in SEQ ID NO: 36 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:36; and the CDRL3 comprises the sequence set forth in SEQ ID NO: 41 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 41.

39. The antibody or antigen binding fragment thereof of any of embodiments 1-29 and 38, wherein the CDRH1 comprises the sequence set forth in SEQ ID NO: 16, the CDRH2 comprises the sequence set forth in SEQ ID NO: 21; the CDRH3 comprises the sequence set forth in SEQ ID NO: 26; the CDRL1 comprises the sequence set forth in SEQ ID NO: 31, the CDRL2 comprises the sequence set forth in SEQ ID NO: 36; and the CDRL3 comprises the sequence set forth in SEQ ID NO: 41.

40. The antibody or antigen binding fragment thereof of any of embodiments 1-29, wherein the CDRH1 comprises the sequence set forth in SEQ ID NO: 15 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 15, the CDRH2 comprises the sequence set forth in SEQ ID NO: 20 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:20; the CDRH3 comprises the sequence set forth in SEQ ID NO: 25 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:25; the CDRL1 comprises the sequence set forth in SEQ ID NO: 28 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:28, the CDRL2 comprises the sequence set forth in SEQ ID NO: 35 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:35; and the CDRL3 comprises the sequence set forth in SEQ ID NO: 40 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 40.

41. The antibody or antigen binding fragment thereof of any of embodiments 1-29 and 40, wherein the CDRH1 comprises the sequence set forth in SEQ ID NO: 15, the CDRH2 comprises the sequence set forth in SEQ ID NO: 20; the CDRH3 comprises the sequence set forth in SEQ ID NO: 25; the CDRL1 comprises the sequence set forth in SEQ ID NO: 28, the CDRL2 comprises the sequence set forth in SEQ ID NO: 35; and the CDRL3 comprises the sequence set forth in SEQ ID NO: 40.

42. The antibody or antigen binding fragment thereof of any of embodiments 1-29, wherein the CDRH1 comprises the sequence set forth in SEQ ID NO: 17 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 17, the CDRH2 comprises the sequence set forth in SEQ ID NO: 22 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:22; the CDRH3 comprises the sequence set forth in SEQ ID NO: 27 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:27; the CDRL1 comprises the sequence set forth in SEQ ID NO: 32 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:32, the CDRL2 comprises the sequence set forth in SEQ ID NO: 37 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO:37; and the CDRL3 comprises the sequence set forth in SEQ ID NO: 42 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 42.

43. The antibody or antigen binding fragment thereof of any of embodiments 1-29 and 42, wherein the CDRH1 comprises the sequence set forth in SEQ ID NO: 17, the CDRH2 comprises the sequence set forth in SEQ ID NO: 22; the CDRH3 comprises the sequence set forth in SEQ ID NO: 27; the CDRL1 comprises the sequence set forth in SEQ ID NO: 32, the CDRL2 comprises the sequence set forth in SEQ ID NO: 37; and the CDRL3 comprises the sequence set forth in SEQ ID NO: 42.

44. The antibody or antigen binding fragment thereof of any of embodiments 1-43, wherein the immunoglobulin heavy chain variable domain comprises the amino acid sequence set forth in any of SEQ ID NOS: 1, 2, 3, 4, 5 or 6, or a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity to any of SEQ ID NOS: 1, 2, 3, 4, 5 or 6.

45. The antibody or antigen binding fragment thereof of any of embodiments 1-44, wherein the immunoglobulin heavy chain variable domain comprises the amino acid sequence set forth in any of SEQ ID NOS: 1, 2, 3, 4, 5 or 6.

46. The antibody or antigen binding fragment thereof of any of embodiments 1-45, wherein the immunoglobulin heavy chain variable domain comprises the amino acid sequence set forth in SEQ ID NO: 1 or a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity to SEQ ID NO:1.

47. The antibody or antigen binding fragment thereof of any of embodiments 1-46, wherein the immunoglobulin heavy chain variable domain comprises the amino acid sequence set forth in SEQ ID NO: 1.

48. The antibody or antigen binding fragment thereof of any of embodiments 1-45, wherein the immunoglobulin heavy chain variable domain comprises the amino acid sequence set forth in SEQ ID NO: 2, or a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity to SEQ ID NO:2.

49. The antibody or antigen binding fragment thereof of any of embodiments 1-45 and 48, wherein the immunoglobulin heavy chain variable domain comprises the amino acid sequence set forth in SEQ ID NO: 2.

50. The antibody or antigen binding fragment thereof of any of embodiments 1-45, wherein the immunoglobulin heavy chain variable domain comprises the amino acid sequence set forth in SEQ ID NO: 3, or a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity to SEQ ID NO:3.

51. The antibody or antigen binding fragment thereof of any of embodiments 1-45 and 50, wherein the immunoglobulin heavy chain variable domain comprises the amino acid sequence set forth in SEQ ID NO: 3.

52. The antibody or antigen binding fragment thereof of any of embodiments 1-45, wherein the immunoglobulin heavy chain variable domain comprises the amino acid sequence set forth in SEQ ID NO: 4, or a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity to SEQ ID NO:4.

53. The antibody or antigen binding fragment thereof of any of embodiments 1-45 and 52, wherein the immunoglobulin heavy chain variable domain comprises the amino acid sequence set forth in SEQ ID NO: 4.

54. The antibody or antigen binding fragment thereof of any of embodiments 1-45, wherein the immunoglobulin heavy chain variable domain comprises the amino acid sequence set forth in SEQ ID NO: 5, or a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity to SEQ ID NO:5.

55. The antibody or antigen binding fragment thereof of any of embodiments 1-45 and 54, wherein the immunoglobulin heavy chain variable domain comprises the amino acid sequence set forth in SEQ ID NO: 5.

56. The antibody or antigen binding fragment thereof of any of embodiments 1-45, wherein the immunoglobulin heavy chain variable domain comprises the amino acid sequence set forth in SEQ ID NO: 6, or a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity to SEQ ID NO:6.

57. The antibody or antigen binding fragment thereof of any of embodiments 1-45 and 56, wherein the immunoglobulin heavy chain variable domain comprises the amino acid sequence set forth in SEQ ID NO: 6.

58. The antibody or antigen binding fragment thereof of any of embodiments 1-57, wherein the immunoglobulin light chain variable domain comprises the amino acid sequence set forth in any of SEQ ID NOS: 7, 8, 9, 10, 11 or 12, or a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity to any of SEQ ID NOS: 7, 8, 9, 10, 11 or 12.

59. The antibody or antigen binding fragment thereof of any of embodiments 1-58, wherein the immunoglobulin light chain variable domain comprises the amino acid sequence set forth in any of SEQ ID NOS: 7, 8, 9, 10, 11 or 12.

60. The antibody or antigen binding fragment thereof of any of embodiments 1-59, wherein the immunoglobulin light chain variable domain comprises the amino acid sequence set forth in SEQ ID NO: 7, or a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity to SEQ ID NO:7.

61. The antibody or antigen binding fragment thereof of any of embodiments 1-60, wherein the immunoglobulin light chain variable domain comprises the amino acid sequence set forth in SEQ ID NO: 7.

62. The antibody or antigen binding fragment thereof of any of embodiments 1-59, wherein the immunoglobulin light chain variable domain comprises the amino acid sequence set forth in SEQ ID NO: 8, or a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity to SEQ ID NO: 8.

63. The antibody or antigen binding fragment thereof of any of embodiments 1-59 and 62, wherein the immunoglobulin light chain variable domain comprises the amino acid sequence set forth in SEQ ID NO: 8.

64. The antibody or antigen binding fragment thereof of any of embodiments 1-59, wherein the immunoglobulin light chain variable domain comprises the amino acid sequence set forth in SEQ ID NO: 9, or a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity to SEQ ID NO: 9.

65. The antibody or antigen binding fragment thereof of any of embodiments 1-59 and 64, wherein the immunoglobulin light chain variable domain comprises the amino acid sequence set forth in SEQ ID NO: 9.

66. The antibody or antigen binding fragment thereof of any of embodiments 1-59, wherein the immunoglobulin light chain variable domain comprises the amino acid sequence set forth in SEQ ID NO: 10, or a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity to SEQ ID NO: 10.

67. The antibody or antigen binding fragment thereof of any of embodiments 1-59 and 66, wherein the immunoglobulin light chain variable domain comprises the amino acid sequence set forth in SEQ ID NO: 10.

68. The antibody or antigen binding fragment thereof of any of embodiments 1-59, wherein the immunoglobulin light chain variable domain comprises the amino acid sequence set forth in SEQ ID NO: 11, or a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity to SEQ ID NO: 11.

69. The antibody or antigen binding fragment thereof of any of embodiments 1-59 and 68, wherein the immunoglobulin light chain variable domain comprises the amino acid sequence set forth in SEQ ID NO: 11.

70. The antibody or antigen binding fragment thereof of any of embodiments 1-59, wherein the immunoglobulin light chain variable domain comprises the amino acid sequence set forth in SEQ ID NO: 12, or a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity to SEQ ID NO: 12.

71. The antibody or antigen binding fragment thereof of any of embodiments 1-59 and 70, wherein the immunoglobulin light chain variable domain comprises the amino acid sequence set forth in SEQ ID NO: 12.

72. The antibody or antigen binding fragment thereof of any of embodiments 1-71, wherein the immunoglobulin heavy chain variable domain comprises the amino acid sequence set forth in any of SEQ ID NOS: 1, 2, 3, 4, 5 or 6 or a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity to any of SEQ ID NOS: 1, 2, 3, 4, 5 or 6, and the immunoglobulin light chain variable domain comprises the amino acid sequence set forth in any of SEQ ID NOS: 7, 8, 9, 10, 11 or 12, or a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity to any of SEQ ID NOS: 7, 8, 9, 10, 11 or 12.

73. The antibody or antigen binding fragment thereof of any of embodiments 1-72, wherein the immunoglobulin heavy chain variable domain comprises the amino acid sequence set forth in any of SEQ ID NOS: 1, 2, 3, 4, 5 or 6, and the immunoglobulin light chain variable domain comprises the amino acid sequence set forth in any of SEQ ID NOS: 7, 8, 9, 10, 11 or 12.

74. The antibody or antigen binding fragment thereof of any of embodiments 1-73, wherein the immunoglobulin heavy chain variable domain comprises the amino acid sequence set forth in SEQ ID NO: 1, or a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity to SEQ ID NO: 1; and the immunoglobulin light chain variable domain comprises the amino acid sequence set forth in SEQ ID NO: 7, or a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity to SEQ ID NO:7.

75. The antibody or antigen binding fragment thereof of any of embodiments 1-74, wherein the immunoglobulin heavy chain variable domain comprises the amino acid sequence set forth in SEQ ID NO: 1, and the immunoglobulin light chain variable domain comprises the amino acid sequence set forth in SEQ ID NO: 7.

76. The antibody or antigen binding fragment thereof of any of embodiments 1-73, wherein the immunoglobulin heavy chain variable domain comprises the amino acid sequence set forth in SEQ ID NO: 2, or a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity to SEQ ID NO: 2; and the immunoglobulin light chain variable domain comprises the amino acid sequence set forth in SEQ ID NOS: 8, or a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity to SEQ ID NO:8.

77. The antibody or antigen binding fragment thereof of any of embodiments 1-73 and 76, wherein the immunoglobulin heavy chain variable domain comprises the amino acid sequence set forth in SEQ ID NO: 2, and the immunoglobulin light chain variable domain comprises the amino acid sequence set forth in SEQ ID NO: 8.

78. The antibody or antigen binding fragment thereof of any of embodiments 1-73, wherein the immunoglobulin heavy chain variable domain comprises the amino acid sequence set forth in SEQ ID NO: 3, or a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity to SEQ ID NO: 3; and the immunoglobulin light chain variable domain comprises the amino acid sequence set forth in SEQ ID NO: 9, or a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity to SEQ ID NO:9.

79. The antibody or antigen binding fragment thereof of any of embodiments 1-73 and 78, wherein the immunoglobulin heavy chain variable domain comprises the amino acid sequence set forth in SEQ ID NO: 3, and the immunoglobulin light chain variable domain comprises the amino acid sequence set forth in SEQ ID NO: 9.

80. The antibody or antigen binding fragment thereof of any of embodiments 1-73, wherein the immunoglobulin heavy chain variable domain comprises the amino acid sequence set forth in SEQ ID NO: 4, or a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity to SEQ ID NO: 4; and the immunoglobulin light chain variable domain comprises the amino acid sequence set forth in SEQ ID NO: 9, or a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity to SEQ ID NO:9.

81. The antibody or antigen binding fragment thereof of any of embodiments 1-73 and 80, wherein the immunoglobulin heavy chain variable domain comprises the amino acid sequence set forth in SEQ ID NO: 4, and the immunoglobulin light chain variable domain comprises the amino acid sequence set forth in SEQ ID NO: 9.

82. The antibody or antigen binding fragment thereof of any of embodiments 1-73, wherein the immunoglobulin heavy chain variable domain comprises the amino acid sequence set forth in SEQ ID NO: 4, or a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity to SEQ ID NO: 4; and the immunoglobulin light chain variable domain comprises the amino acid sequence set forth in SEQ ID NO: 10, or a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity to SEQ ID NO:10.

83. The antibody or antigen binding fragment thereof of any of embodiments 1-73 and 82, wherein the immunoglobulin heavy chain variable domain comprises the amino acid sequence set forth in SEQ ID NO: 4, and the immunoglobulin light chain variable domain comprises the amino acid sequence set forth in SEQ ID NO: 10.

84. The antibody or antigen binding fragment thereof of any of embodiments 1-73, wherein the immunoglobulin heavy chain variable domain comprises the amino acid sequence set forth in SEQ ID NO: 5, or a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity to SEQ ID NO: 5; and the immunoglobulin light chain variable domain comprises the amino acid sequence set forth in SEQ ID NO: 11, or a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity to SEQ ID NO:11.

85. The antibody or antigen binding fragment thereof of any of embodiments 1-73 and 84, wherein the immunoglobulin heavy chain variable domain comprises the amino acid sequence set forth in SEQ ID NO: 5, and the immunoglobulin light chain variable domain comprises the amino acid sequence set forth in SEQ ID NO:11.

86. The antibody or antigen binding fragment thereof of any of embodiments 1-73, wherein the immunoglobulin heavy chain variable domain comprises the amino acid sequence set forth in SEQ ID NO: 6, or a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity to SEQ ID NO: 6; and the immunoglobulin light chain variable domain comprises the amino acid sequence set forth in SEQ ID NO: 12, or a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity to SEQ ID NO:12.

87. The antibody or antigen binding fragment thereof of any of embodiments 1-73 and 86, wherein the immunoglobulin heavy chain variable domain comprises the amino acid sequence set forth in SEQ ID NO: 6, and the immunoglobulin light chain variable domain comprises the amino acid sequence set forth in SEQ ID NO: 12.

88. The antibody or antigen binding fragment thereof of any of embodiments 1-87, comprising one immunoglobulin heavy chain variable domain and one immunoglobulin light chain variable domain.

89. The antibody or antigen binding fragment thereof of any of embodiments 1-88, comprising two immunoglobulin heavy chain variable domains and two immunoglobulin light chain variable domains.

90. The antibody or antigen binding fragment thereof of any of embodiments 1-89, wherein the antibody or antigen binding fragment thereof is isolated.

91. The antibody or antigen binding fragment thereof of any of embodiments 1-90, wherein the antibody or antigen binding fragment thereof is humanized.

92. The antibody or antigen binding fragment thereof of any of embodiments 1-91, wherein the antibody or antigen binding fragment thereof is conjugated.

93. The antibody or antigen binding fragment thereof of any of embodiments 1-92, further comprising an oligonucleotide.

94. The antibody or antigen binding fragment thereof of embodiment 93, wherein the oligonucleotide comprises a sample barcode sequence.

95. The antibody or antigen binding fragment thereof of embodiment 93 or embodiment 94, wherein the oligonucleotide comprises a binding site for a primer and an anchor.

96. The antibody or antigen binding fragment thereof of any of embodiments 1-95, wherein the antibody or antigen binding fragment thereof is conjugated to a detectable marker or label.

97. The antibody or antigen binding fragment thereof of embodiment 96, wherein the detectable marker or label is conjugated directly to the antigen or antigen binding fragment thereof.

98. The antibody or antigen binding fragment thereof of embodiment 96, wherein the detectable marker or label is conjugated to the oligonucleotide.

99. The antibody or antigen binding fragment thereof of any of embodiments 96-98, wherein the detectable marker or label comprises a detectable moiety.

100. The antibody or antigen binding fragment thereof of embodiment 99, wherein the detectable moiety is a radioisotope, fluorescent label or enzyme-substrate label.

101. The antibody or antigen binding fragment thereof of any of embodiments 1-100, wherein the antibody or antigen binding fragment thereof is non-diffusively immobilized on a solid support.

102. The antibody or antigen binding fragment thereof of any of embodiments 1-101, that is a single chain fragment.

103. The antibody or antigen binding fragment thereof of embodiment 102, wherein the single chain fragment is a single chain variable fragment (scFv).

104. The antibody or antigen binding fragment thereof of any of embodiments 1-103, for use in the detection of CD28 in a sample.

105. The antibody or antigen binding fragment thereof of any of embodiments 1-104, wherein the antibody or antigen binding fragment thereof binds to a cell expressing CD28 in a sample.

106. The antibody or antigen binding fragment thereof of embodiment 104 or embodiment 105, wherein the sample comprises immune cells.

107. The antibody or antigen binding fragment thereof of any of embodiments 104-106, wherein the sample comprises a heterogenous population of immune cells.

108. The antibody or antigen binding fragment thereof of embodiment 106 or embodiment 107, wherein the immune cell is selected from B cells, plasmacytoid dendritic cells (pDCs), lymphocytes, leukocytes, T cells, monocytes, macrophages, neutrophils, myeloid dendritic cells (mDCs), innate lymphoid cells, mast cells, eosinophils, basophils, natural killer cells, and peripheral blood mononuclear cells (PBMCs).

109. The antibody or antigen binding fragment thereof of any of embodiments 104-108, wherein the sample comprises a cell with a disease or disorder.

110. The antibody or antigen binding fragment thereof of embodiment 109, wherein the disease or disorder is a cancer, an autoimmune disorder, an inflammatory disorder, a neurologic disorder, or an infection.

111. The antibody or antigen binding fragment thereof of embodiment 110, wherein the cancer is acute myeloid leukemia, acute lymphoblastic leukemia, colorectal, ovarian, gynecologic, liver, glioblastoma, Hodgkin lymphoma, chronic lymphocytic leukemia, esophagus, gastric, pancreas, colon, kidney, head and neck, lung and melanoma.

112. The antibody or antigen binding fragment thereof of any of embodiments 104-111, wherein the detection comprises the use of a single antibody or antigen binding fragment thereof to bind a portion of CD28

113. The antibody or antigen binding fragment thereof of any of embodiments 104-112, wherein the detection comprises the use of two antibody or antigen binding fragments thereof, each capable of binding to a different portion of CD28.

114. The antibody or antigen binding fragment thereof of any of embodiments 104-113, wherein the detection of CD28 is on the surface of a cell.

115. The antibody or antigen binding fragment thereof of any of embodiments 104-114, wherein the detection of CD28 is intracellular.

116. The antibody or antigen binding fragment thereof of any of embodiments 104-115, wherein the detection of CD28 indicates the presence or absence of a disease or disorder.

117. The antibody or antigen binding fragment thereof of any of embodiments 104-116, wherein the detection is performed in vitro.

118. The antibody or antigen binding fragment thereof of any of embodiments 104-116, wherein the detection is performed in vivo.

119. The antibody or antigen binding fragment thereof of any of embodiments 1-118, wherein the antibody or antigen binding fragment thereof binds to a CD28 expressing cell, and activates a T cell.

120. The antibody or antigen binding fragment thereof of embodiment 119, wherein the T cell activation comprises a first signal induced via a TCR and a second signal induced by binding of the CD28 to B7-1 (also known as CD80) or B7-2 (also known as CD86).

121. The antibody or antigen binding fragment thereof of embodiment 119, wherein the T cell activation does not comprise a first signal induced via a TCR.

122. The antibody or antigen binding fragment thereof of any of embodiments 104-121, wherein the antibody or antigen binding fragment thereof bound to the CD28 expressing cell enhances the production of cytokines.

123. The antibody or antigen binding fragment thereof of any of embodiments 104-122, wherein the antibody or antigen binding fragment thereof bound to the CD28 expressing cell initiates differentiation of the cell into a memory phenotype

124. A diagnostic antibody or antigen binding fragment thereof comprising the antibody or antigen binding fragment thereof of any of embodiments 1-123.

125. A kit comprising the antibody or antigen binding fragment thereof of any one of embodiments 1-123 or the diagnostic antibody or antigen binding fragment thereof of embodiment 124.

126. The kit of embodiment 125, comprising a diagnostics kit configured to detect CD28 in a biological sample.

127. A composition comprising the antibody or antigen binding fragment thereof of any of embodiments 1-123, and a pharmaceutically acceptable excipient.

128. The composition of embodiment 127, wherein the antibody or antigen binding fragment thereof of is used as an adjuvant or in conjunction with an adjuvant.

129. An isolated nucleic acid comprising a nucleotide sequence that encodes the immunoglobulin heavy chain variable domain of the agent of any of embodiments 1-123.

130. The isolated nucleic acid of embodiment 129, wherein the immunoglobulin heavy chain variable domain comprises the sequence of nucleotides set forth in any of SEQ ID NOs: 43-50.

131. The isolated nucleic acid of embodiment 129 or embodiment 130, wherein the immunoglobulin heavy chain variable domain comprises the sequence of nucleotides set forth in SEQ ID NO: 43.

132. The isolated nucleic acid of embodiment 129 or embodiment 130, wherein the immunoglobulin heavy chain variable domain comprises the sequence of nucleotides set forth in SEQ ID NO: 44.

133. The isolated nucleic acid of embodiment 129 or embodiment 130, wherein the immunoglobulin heavy chain variable domain comprises the sequence of nucleotides set forth in SEQ ID NO: 45.

134. The isolated nucleic acid of embodiment 129 or embodiment 130, wherein the immunoglobulin heavy chain variable domain comprises the sequence of nucleotides set forth in SEQ ID NO: 46.

135. The isolated nucleic acid of embodiment 129 or embodiment 130, wherein the immunoglobulin heavy chain variable domain comprises the sequence of nucleotides set forth in SEQ ID NO: 47.

136. The isolated nucleic acid of embodiment 129 or embodiment 130, wherein the immunoglobulin heavy chain variable domain comprises the sequence of nucleotides set forth in SEQ ID NO: 48.

137. The isolated nucleic acid of embodiment 129 or embodiment 130, wherein the immunoglobulin heavy chain variable domain comprises the sequence of nucleotides set forth in SEQ ID NO: 49.

138. The isolated nucleic acid of embodiment 129 or embodiment 130, wherein the immunoglobulin heavy chain variable domain comprises the sequence of nucleotides set forth in SEQ ID NO: 50.

139. An isolated nucleic acid comprising a nucleotide sequence that encodes the immunoglobulin light chain variable domain of the agent of any of embodiments 1-123.

140. The isolated nucleic acid molecule of embodiment 139, wherein the immunoglobulin light chain variable domain comprises the sequence of nucleotides set forth in any of SEQ ID NOs: 51-58.

141. The isolated nucleic acid of embodiment 139 or embodiment 140, wherein the immunoglobulin light chain variable domain comprises the sequence of nucleotides set forth in SEQ ID NO: 51.

142. The isolated nucleic acid of embodiment 139 or embodiment 140, wherein the immunoglobulin light chain variable domain comprises the sequence of nucleotides set forth in SEQ ID NO: 52.

143. The isolated nucleic acid of embodiment 139 or embodiment 140, wherein the immunoglobulin light chain variable domain comprises the sequence of nucleotides set forth in SEQ ID NO: 53.

144. The isolated nucleic acid of embodiment 139 or embodiment 140, wherein the immunoglobulin light chain variable domain comprises the sequence of nucleotides set forth in SEQ ID NO: 54.

145. The isolated nucleic acid of embodiment 139 or embodiment 140, wherein the immunoglobulin light chain variable domain comprises the sequence of nucleotides set forth in SEQ ID NO: 55.

146. The isolated nucleic acid of embodiment 139 or embodiment 140, wherein the immunoglobulin light chain variable domain comprises the sequence of nucleotides set forth in SEQ ID NO: 56.

147. The isolated nucleic acid of embodiment 139 or embodiment 140, wherein the immunoglobulin light chain variable domain comprises the sequence of nucleotides set forth in SEQ ID NO: 57.

148. The isolated nucleic acid of embodiment 139 or embodiment 140, wherein the immunoglobulin light chain variable domain comprises the sequence of nucleotides set forth in SEQ ID NO: 58.

149. An isolated nucleic acid comprising a nucleotide sequence that encodes the immunoglobulin heavy chain variable domain and the immunoglobulin light chain variable domain of the antibody or antigen binding fragment thereof of any of embodiments 1-123.

150. The isolated nucleic acid of any of embodiments 129-149, wherein the nucleotide sequence that encodes the immunoglobulin heavy chain variable domain comprises the sequence set forth in any of SEQ ID NOs: 43-50; and the immunoglobulin light chain variable domain comprises the sequence of amino acids set forth in any of SEQ ID NOs: 51-58.

151. The isolated nucleic acid of embodiment 150, wherein the nucleotide sequence that encodes the immunoglobulin heavy chain variable domain comprises the sequence set forth in SEQ ID NO: 43; and the immunoglobulin light chain variable domain comprises the sequence of amino acids set forth in SEQ ID NO: 51.

152. The isolated nucleic acid of embodiment 150, wherein the nucleotide sequence that encodes the immunoglobulin heavy chain variable domain comprises the sequence set forth in SEQ ID NO: 44; and the immunoglobulin light chain variable domain comprises the sequence of amino acids set forth in SEQ ID NO: 52.

153. The isolated nucleic acid of embodiment 150, wherein the nucleotide sequence that encodes the immunoglobulin heavy chain variable domain comprises the sequence set forth in SEQ ID NO: 45; and the immunoglobulin light chain variable domain comprises the sequence of amino acids set forth in SEQ ID NO: 53.

154. The isolated nucleic acid of embodiment 150, wherein the nucleotide sequence that encodes the immunoglobulin heavy chain variable domain comprises the sequence set forth in SEQ ID NO: 46; and the immunoglobulin light chain variable domain comprises the sequence of amino acids set forth in SEQ ID NO: 54.

155. The isolated nucleic acid of embodiment 150, wherein the nucleotide sequence that encodes the immunoglobulin heavy chain variable domain comprises the sequence set forth in SEQ ID NO: 47; and the immunoglobulin light chain variable domain comprises the sequence of amino acids set forth in SEQ ID NO: 55.

156. The isolated nucleic acid of embodiment 150, wherein the nucleotide sequence that encodes the immunoglobulin heavy chain variable domain comprises the sequence set forth in SEQ ID NO: 48; and the immunoglobulin light chain variable domain comprises the sequence of amino acids set forth in SEQ ID NO: 56.

157. The isolated nucleic acid of embodiment 150, wherein the nucleotide sequence that encodes the immunoglobulin heavy chain variable domain comprises the sequence set forth in SEQ ID NO: 49; and the immunoglobulin light chain variable domain comprises the sequence of amino acids set forth in SEQ ID NO: 57.

158. The isolated nucleic acid of embodiment 150, wherein the nucleotide sequence that encodes the immunoglobulin heavy chain variable domain comprises the sequence set forth in SEQ ID NO:50; and the immunoglobulin light chain variable domain comprises the sequence of amino acids set forth in SEQ ID NO: 58.

159. A recombinant expression vector comprising the isolated nucleic acid of any of embodiments 129-158.

160. A recombinant expression vector comprising a first expression cassette and a second expression cassette, wherein the first expression cassette comprises a nucleic acid molecule comprising a nucleotide sequence that encodes the immunoglobulin heavy chain variable domain of any one of embodiments 1-123, and the second expression cassette comprises a nucleic acid molecule comprising a nucleotide sequence that encodes an immunoglobulin light chain variable domain of the antibody or antigen binding fragment thereof of any one of embodiments 1-123.

161. A recombinant expression vector comprising a first expression cassette and a second expression cassette, wherein the first expression cassette comprises a nucleic acid molecule comprising the nucleotide sequence of any of embodiments 123-158, and the second expression cassette comprises a nucleic acid molecule comprising the nucleotide sequence of any of embodiments 123-158.

162. The recombinant expression vector of any of embodiments 159-161, wherein the first and second expression cassettes comprise a promoter.

163. A host cell transfected with the recombinant expression vector of any of embodiments 159-162.

164. An agent-drug conjugate comprising antibody or antigen binding fragment thereof of any of embodiments 1-123.

165. A composition comprising the antibody-drug conjugate of embodiment 164, and a pharmaceutically acceptable carrier.

166. A method of detecting CD28, comprising:
a) contacting a sample with the antibody or antigen binding fragment thereof of any of embodiments 1-123, under conditions to bind said antibody or antigen binding fragment thereof to a CD28 receptor on said sample, wherein the binding generates the production of a receptor/antibody or antigen binding fragment thereof of complex;
b) detecting the presence of the receptor/antibody or antigen binding fragment thereof of complexes;
c) wherein the detecting comprises the presence or absence of the CD28 receptor on said sample.

167. A method of treating or preventing a disease or disorder associated with CD28 in a subject, comprising:
a) contacting a sample known or suspected to contain CD28 with the antibody or antigen binding fragment thereof of any of embodiments 1-123,
b) detecting the presence of complexes comprising CD28 and the antibody or antigen binding fragment thereof; wherein the presence of the complexes indicates the presence of a disease or disorder; and
c) administering to the subject the antibody or antigen binding fragment thereof of any of embodiments 1-123.

168. A method of diagnosing a disease or disorder, comprising:
a) isolating a sample from a subject
b) incubating the sample with the antibody or antigen binding fragment thereof of any of embodiments 1-123, for a period of time sufficient to generate CD28: anti-CD28 complexes;
c) detecting the presence or absence of the CD28: anti-CD28 complexes from the isolated tissue, and
d) associating presence or abundance of CD28 with a location of interest of a tissue sample.

169. The method of embodiment 168, wherein the increase of CD28 over a control level in the location of interest of the tissue sample is indicative of a disease or disorder in a subject.

170. The method of any of embodiments 166-169, wherein the method is performed in vitro.

171. The method of any of embodiments 166-169, wherein the method is performed in vivo.

172. The method of any of embodiments 166-171, wherein the detection comprises intracellular detection.

173. The method of any of embodiments 166-172, wherein the detection comprises detection on the surface of a cell.

174. The method of any of embodiments 166-173, wherein the detection comprises hybridization of a detectable moiety to the antibody or antigen binding fragment thereof.

175. The method of any of embodiments 166-174, wherein the sample is contacted with a second antibody.

176. The method of any of embodiments 166-175, wherein the second antibody is an antibody comprising a detectable moiety.

177. The method of any of embodiments 166-176, wherein the detectable moiety comprises an oligonucleotide.

178. The method of any of embodiments 166-177, wherein the detectable moiety comprises a fluorescent label.

179. The method of any of embodiments 166-178, wherein the measurement comprises sequencing.

180. The method of any of embodiments 166-179, wherein the detectable moiety comprises immunofluorescence.

181. The method of any of embodiments 166-180, wherein sample is a formalin-fixed paraffin-embedded sample.

182. The method of any of embodiments 166-181, wherein the sample comprises a cell.

183. The method of any of embodiments 166-182, wherein the sample comprises a tissue sample.

184. The method of any of embodiments 166-183, wherein the sample comprises immune cells.

185. The method of embodiment 184, wherein the immune cell is selected from B cells, plasmacytoid dendritic cells (pDCs), lymphocytes, leukocytes, T cells, monocytes, macrophages, neutrophils, myeloid dendritic cells (mDCs), innate lymphoid cells, mast cells, eosinophils, basophils, natural killer cells, and peripheral blood mononuclear cells (PBMCs).

186. The method of any of embodiments 166-185, wherein the sample comprises a tissue or cells associated with a disease or disorder.

187. The method of any of embodiments 166-186, wherein the disease or disorder is a cancer, an autoimmune disorder, an inflammatory disorder, or an infection.

188. The method of embodiment 186 or embodiment 187, wherein the disease or disorder is chosen from non-viral cancers, virus-associated cancers, cancers associated with HBV infection, cancers associated with Epstein-Barr virus (EBV) infection, cancers associated with polyomavirus infection, erythema nodosum leprosum (ENL), autoimmune diseases, autoimmune inflammation, autoimmune thyroid diseases, B-cell lymphoma, T-cell lymphoma, acute myeloid leukemia, Hodgkin's Disease, acute myelogenous leukemia, acute myelomonocytic leukemia, acute lymphoblastic leukemia, chronic myelogenous leukemia, chronic lymphocytic leukemia, B cell large cell lymphoma, malignant lymphoma, acute leukemia, lymphosarcoma cell leukemia, B-cell leukemias, myelodysplastic syndromes, solid phase cancer, herpes viral infections, and/or rejection of transplanted tissues or organs.

189. The antibody or antigen binding fragment thereof of any of embodiments 1-123, for use in a method of associating presence or abundance of CD28 with a location of interest of a tissue sample.

190. The antibody or antigen binding fragment thereof of any of embodiments 1-123, for use in a method of detecting CD28 in a tissue sample

191. The antibody or antigen binding fragment thereof of embodiments 189 or embodiment 190, wherein the method comprises generating a nucleic acid molecule comprising all or a portion of the sequence of the oligonucleotide or a complement thereof.

192. The antibody or antigen binding fragment thereof of any of embodiments 1-123, for use in the construction of a protein library.

193. The antibody or antigen binding fragment thereof of embodiment 192, wherein the construction of a protein library comprises sequencing.

194. The antibody or antigen binding fragment thereof of embodiment 192, wherein the construction of a protein library comprises the use of flow cytometry.

### EXAMPLES

### Example 1. Generation Anti-CD28 Antibody Expressing Hybridomas

This Example describes the generation and characterization of hybridomas that secrete monoclonal antibodies that react with CD28 using murine models.

Briefly, mice were immunized with a CD28 immunogen, and hybridomas were formed using standard protocols to fuse myeloma cells with spleens, and lymph node cells were drained and harvested. Successful fusions were selected into HAT medium, and cloned into approximately one cell per well in microtiter plates, after which culture supernatants were tested against CD28-expressing cell transfectants by flow cytometry. Wells were selected by assessment of staining profiles and then sub-cultured into larger vessels and subcloned. Hybridoma sub-clones were further characterized by flow cytometry using CD28-transfected cells. Candidate clones expressing exemplary anti-CD28 antibodies were selected and screened using various methods, including by flow cytometry against human blood cells divided into distinct subsets (e.g., lymphocytes, monocytes, and the like), and against one or more cell lines generated from diseased and/or infected human cells. The percentage of positive cells in each blood cell subset was quantified as compared to isotype control.

### Example 2. Sequencing of Exemplary Anti-CD28 Antibody Variable Regions

In This Example describes the sequencing of exemplary anti-CD28 antibodies generated in Example 1 above.

In Cells from anti-CD28 hybridoma cell lines described in Example 1 above were grown in standard mammalian tissue culture media. Total RNA was isolated from hybridoma cells from various clones expressing anti-CD28 monoclonal antibodies using a procedure based on the RNeasy Mini Kit (Qiagen). Briefly, RNA was used to generate a first strand of cDNA of the light chain and heavy chain variable domains. Both light chain and heavy chain variable domain cDNAs were amplified by a 5′-RACE technique, and positive clones were prepared by PCR and then subjected to DNA sequencing.

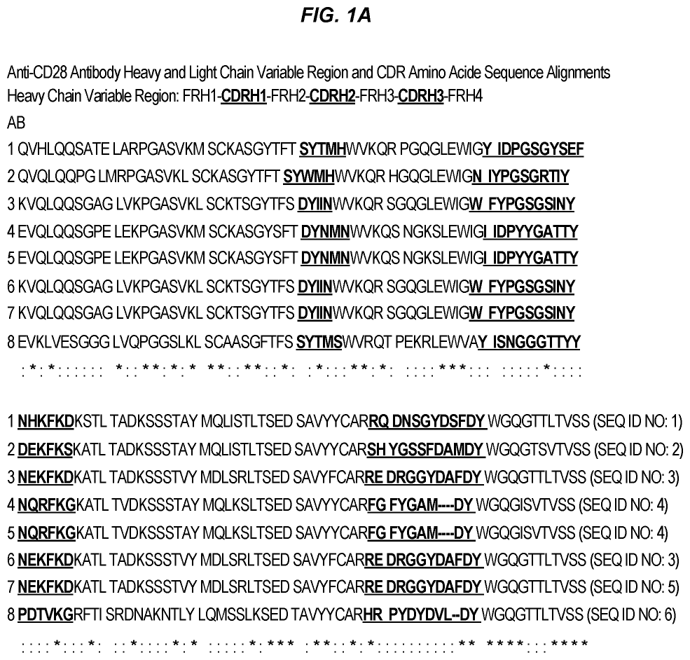

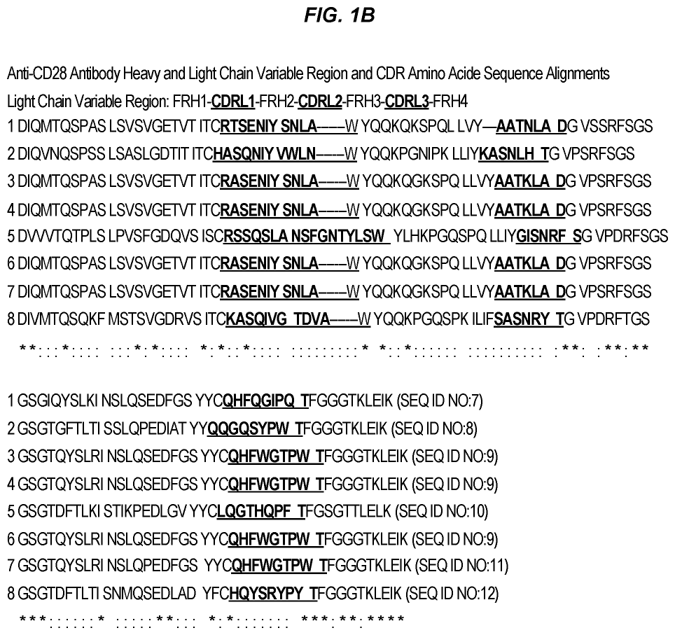

In Amino acid sequences of the individual variable domains (CDRs and Framework regions), including the CDR1, CDR2, and CDR3 regions, for both the heavy and light chains for eight different antibodies (clones), designated AB 1-8 (also referred to herein as antibodies 1-8, and clones 1-8), are shown in FIGS. 1A and 1B. The various heavy and light chain CDR sequences are shown in Table E1, below.

TABLE E1
CDR Sequences and SEQ ID NOs. of VH and VL
domains of exemplary anti-CD28 antibodies.

| SEQ ID | CDR | AB (clone) | Amino Acid |
|---|---|---|---|
| NO | Type | Number(s) | Sequence |
| 13 | CDRH1 | 1 | SYTMH |
| 14 | CDRH1 | 2 | SYWMH |
| 15 | CDRH1 | 3, 6, 7 | DYIIN |
| 16 | CDRH1 | 4, 5 | DYNMN |
| 17 | CDRH1 | 8 | SYTMS |
| 18 | CDRH2 | 1 | YIDPGSGYSEFNHKFKD |
| 19 | CDRH2 | 2 | NIYPGSGRTIYDEKFKS |
| 20 | CDRH2 | 3, 6, 7 | WFYPGSGSINYNEKFKD |
| 21 | CDRH2 | 4, 5 | IIDPYYGATTYNQRFKG |
| 22 | CDRH2 | 8 | YISNGGGTTYYPDTVKG |
| 23 | CDRH3 | 1 | RQDNSGYDSFDY |
| 24 | CDRH3 | 2 | SHYGSSFDAMDY |
| 25 | CDRH3 | 3, 6, 7 | REDRGGYDAFDY |
| 26 | CDRH3 | 4, 5 | FGFYGAMDY |
| 27 | CDRH3 | 8 | HRPYDYDVLDY |
| 28 | CDRL1 | 1, 7 | RTSENIYSNLA |
| 29 | CDRL1 | 2 | HASQNIYVWLN |
| 30 | CDRL1 | 3, 4, 6 | RASENIYSNLA |
| 31 | CDRL1 | 5 | RSSQSLANSFGNTYLS |
| 32 | CDRL1 | 8 | KASQIVGTDVA |
| 33 | CDRL2 | 1 | AATNLAD |
| 34 | CDRL2 | 2 | KASNLHT |
| 35 | CDRL2 | 3, 4, 6, 7 | AATKLAD |
| 36 | CDRL2 | 5 | GISNRFS |
| 37 | CDRL2 | 8 | SASNRYT |
| 38 | CDRL3 | 1 | QHFWGIPWT |
| 39 | CDRL3 | 2 | QQGQSYPWT |
| 40 | CDRL3 | 3, 4, 6, 7 | QHFWGTPWT |
| 41 | CDRL3 | 5 | LQGTHQPFT |
| 42 | CDRL3 | 8 | HQYSRYPYT |

### Example 3. Detection of CD28 Expressing Cells Using Exemplary Anti-CD28 Antibodies

This Example describes the ability of exemplary generated anti-CD28 antibodies to detect fresh or fixed cells expressing CD28, assessed by flow cytometry.

### A) Surface Staining of Fresh Cells

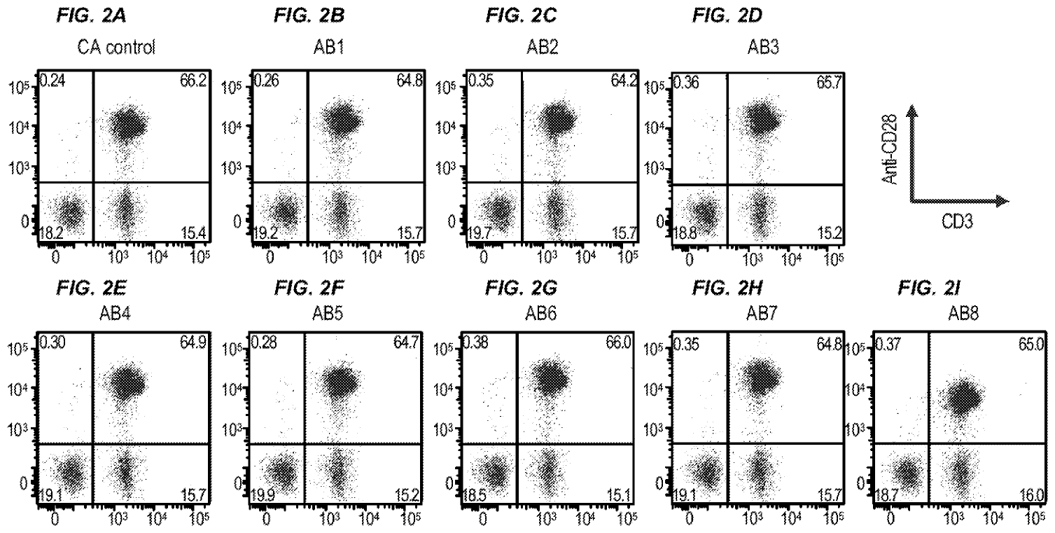

Fresh human peripheral blood cells were obtained by venous puncture, and cells were incubated with FITC labeled anti-human CD3 antibody (UCHT1; Cat. No: 319001) and exemplary anti-CD28 antibodies conjugated to PE in the dark for 15 minutes. A commercially available anti-CD28 antibody (CA control) conjugated to PE was used as control. Cells were lysed with RBC lysis buffer, washed, and resuspended in Fluorofix solution (containing 1% PFA). Data were acquired on a BD Canto II instrument, and analyzed by FlowJo. T cells were gated based on CD28 and CD3 staining patterns (FIGS. 2A-2I). A histogram was generated of each cell population to quantify median fluorescence intensity (MFI), and the MFI of each exemplary antibody and respective control was used to calculate the signal to noise (S:N) ratio (Table E2).

As shown in FIGS. 2A-2I, all exemplary tested anti-CD28 antibodies were capable of detecting CD28 expressing T cells, as measured by number of cells positive for CD28 and CD3. In addition, exemplary antibodies AB1-AB7 demonstrated increased signal to noise ratios over the commercially available antibody used as control, as shown in Table E2.

All reagents used were form BioLegend unless noted otherwise.

TABLE E2
MFI and Signal to Noise rations for exemplary antibodies

| | % CD28+ | % CD28− | MFI CD28+ | MFI CD28− | S/N |
|---|---|---|---|---|---|
| | population | population | population | population | Ratio |
| CA | 66.20% | 15.40% | 9748 | 77 | 173.7 |
| Control | | | | | |
| AB1 | 64.80% | 15.70% | 12615 | 70.5 | 212 |
| AB2 | 64.20% | 15.70% | 13114 | 77.4 | 206.8 |
| AB3 | 65.70% | 15.20% | 15573 | 78 | 259.6 |
| AB4 | 64.90% | 15.70% | 11947 | 73.7 | 192.7 |
| AB5 | 64.70% | 15.20% | 12457 | 78.1 | 194.9 |
| AB6 | 66.00% | 15.10% | 15467 | 80.9 | 248.7 |
| AB7 | 64.80% | 15.70% | 16178 | 76.6 | 248.9 |
| AB8 | 65.00% | 16.00% | 5210 | 77.5 | 83.2 |

### B) Surface Staining of Fixed Cells

In a similar experiment, fresh human peripheral blood cells were obtained by venous puncture, treated with RBC lysis buffer and washed with PBS. Cells were left either un-fixed or were fixed with 4% PFA for 15 minutes and washed with PBS to remove PFA. Cells were then counted and adjusted in Cell Staining Buffer and stained with exemplary anti-CD28 antibodies conjugated to PE for 30 minutes in the dark. A commercially available (CA control) anti-CD28 antibody conjugated to PE was used as control. Data were acquired on a BD Canto II instrument, and analyzed by FlowJo.

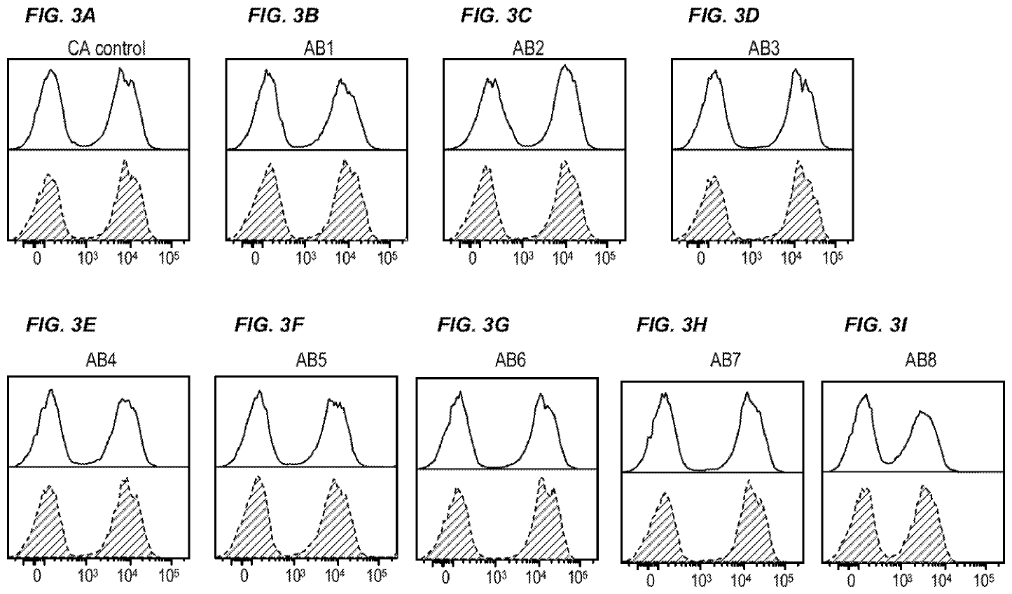

In FIGS. 3A-3I depict histogram overlays for fixed (top) and fresh (bottom) cells labeled with control or exemplary anti-CD28 antibodies AB1-AB8. As shown in FIGS. 3A-31, staining performance was not affected by fixation. In addition, as shown in Table E3, exemplary antibodies AB1-AB7 demonstrated increased signal to noise ratios in both fixed and un-fixed cells over the commercially available antibody used as control.

TABLE E3
Signal to Noise rations for fixed and un-fixed
cells using exemplary antibodies

| | S/N on | S/N on |
|---|---|---|
| | un-fixed | fixed |
| | cells | cells |
| CA control | 79.3 | 52.7 |
| AB1 | 89.2 | 61.6 |
| AB2 | 89 | 50.1 |
| AB3 | 132.8 | 100.8 |
| AB4 | 75.4 | 57.7 |
| AB5 | 94.5 | 63.3 |
| AB6 | 128.4 | 101 |
| AB7 | 130.3 | 107.6 |
| AB8 | 30.9 | 24.6 |

These results demonstrate the ability of exemplary anti-CD28 antibodies to recognize and detect fresh and fixed CD28 expressing cells at high signal to noise ratios.

### Example 4. Assessment of Functional Activity of Exemplary Anti-CD28 Antibodies

This Example describes assessment of the functional activity of exemplary anti-CD28 antibodies by evaluation of activation and expansion of peripheral blood mononuclear cells (PBMCs), with and without anti-CD3 co-stimulation.

To isolate human peripheral blood mononuclear cells (PBMCs), whole blood from healthy volunteers was collected in heparinized cell preparation tubes. The blood was diluted with phosphate buffered saline (PBS) in 2-4 times of volume. The diluted blood suspension (35 mL) was layered carefully over 15 mL of Lymphopure™ in a 50 mL conical tube, and centrifuged at 400×g for 30 to 40 min in a swinging bucket rotor without brake. The mononuclear cell layer was transferred to a fresh 50 mL conical tube, mixed with PBS and centrifuged at 300×g for 5 minutes. The supernatant was removed and the pellet was washed twice with PBS, and centrifuged. The resulting pellet was resuspended in fluorescence activated cell sorting (FACS) buffer. Total T cells were isolated using Mojo Sort human T cell isolation kit with negative selection according to manufacturer's protocol (BioLegend). Cells were then labeled with intracellular fluorescent label carboxyfluorescin succinimidyl ester (CFSE) to monitor cell division, and assessed as described below with (A) or without (B) anti-CD3 co-stimulation.

### A) With Anti-CD3 Co-Stimulation

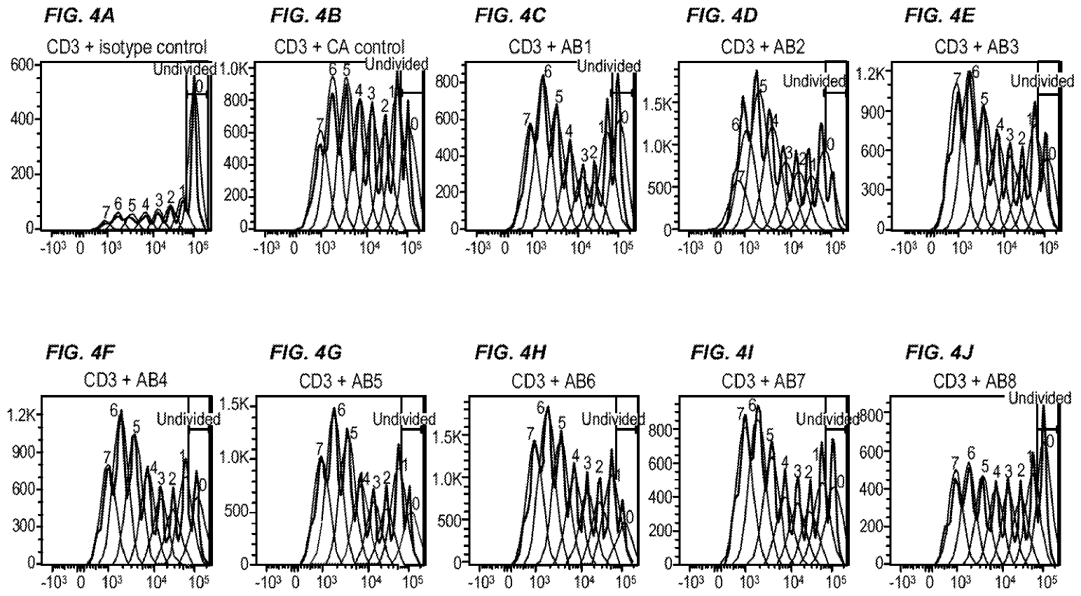

Exemplary anti-CD28 antibodies were assessed for their ability to activate and expand T cells in the presence of the co-stimulating T cell antibody UCHT1 (anti-CD3). CFSE labeled T cells were incubated were plated onto anti-CD3 plus isotype control or anti-CD3 plus anti-CD28 coated 12-well plates and incubated for 4 days. After incubation, cells were acquired on a BD Canto II and corresponding data was analyzed by FlowJo. Results are shown in FIGS. 4A-4J, and quantification of results shown in Table E4.

As shown in FIGS. 4A-4J, whereas anti-CD3 alone (FIG. 4A) produced low rates of cell division, all exemplary antibodies tested were capable to enhancing T cell division, with exemplary antibody AB6 demonstrating the highest division index. These results demonstrate the ability of exemplary tested antibodies to enhance CD3-associated T cell activation and proliferation.

TABLE E4
Analysis of co-stimulation dependent T cell division
using exemplary antibodies

| | Percent | Division | Expansion |
|---|---|---|---|
| | Divided | Index | Index |
| CD3 + Isotype control | 16.9 | 0.29 | 1.7 |
| CD3 + CA control | 54.3 | 1.04 | 4.37 |
| CD3 + AB1 | 42 | 0.8 | 4.03 |
| CD3 + AB2 | 44 | 0.99 | 4.56 |
| CD3 + AB3 | 55.7 | 1.08 | 5.11 |
| CD3 + AB4 | 53.8 | 1.08 | 5.07 |
| CD3 + AB5 | 60.5 | 1.17 | 5.39 |
| CD3 + AB6 | 65.8 | 1.32 | 6.04 |
| CD3 + AB7 | 48 | 0.96 | 4.82 |
| CD3 + AB8 | 40 | 0.76 | 3.51 |

In a similar experiment, PBMC's isolated and subjected to CD3+ isolation (MojoSort Human CD3 selection kit) as described above were plated at 1 million/mL in IMDM media (Thermo Fisher) combined with 200 IU/mL of recombinant human IL-2 (791904) and 5% human AB serum (Gemini), and incubated with 2 μg/mL of anti-human CD3 antibody (OKT3, BioLegend catalog #317326) and 2.5 ug/mL of exemplary anti-human CD28 antibodies. A commercially available clone (CA control) was used as positive control; and the absence of a CD28 antibody was used as a negative control). Cells were incubated for 3-4 days, after which the cells were counted using a ViCell Blu system (Beckman Coulter) and diluted to 0.5 million cells/mL every two-three days, for a period of 10-11 days.

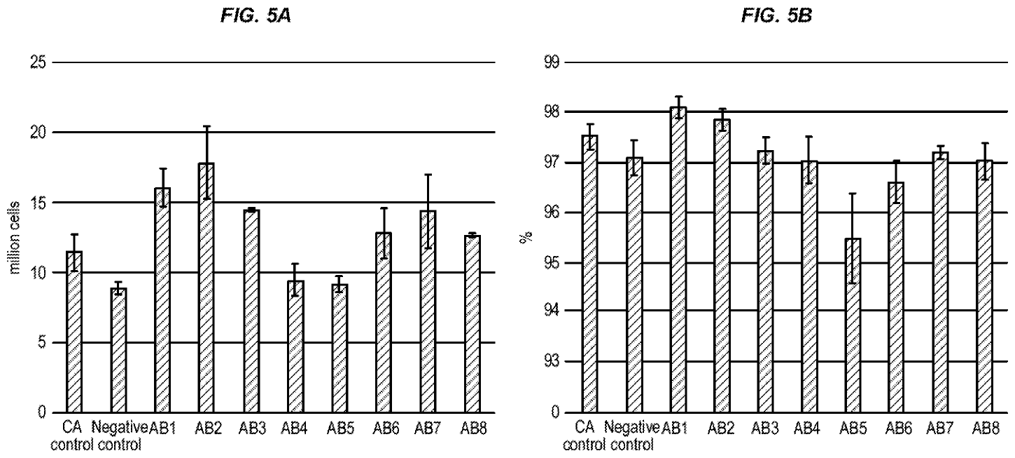

Growth and viability of T cells was evaluated over the course of 11 days. As shown in FIG. 5A, exemplary antibodies AB1, AB2, AB3 and AB6, AB7 and AB8 demonstrated increased T cell at day 11 compared to positive control, with almost all exemplary antibodies demonstrating comparable viability at day 11 compared to control (FIG. 5B). Data are representative of 3 independent experiments.

These results demonstrate the ability of exemplary tested antibodies to enhance T cell co-stimulation with different anti-CD3 antibodies.

### B) Without Anti-CD3 Co-Stimulation

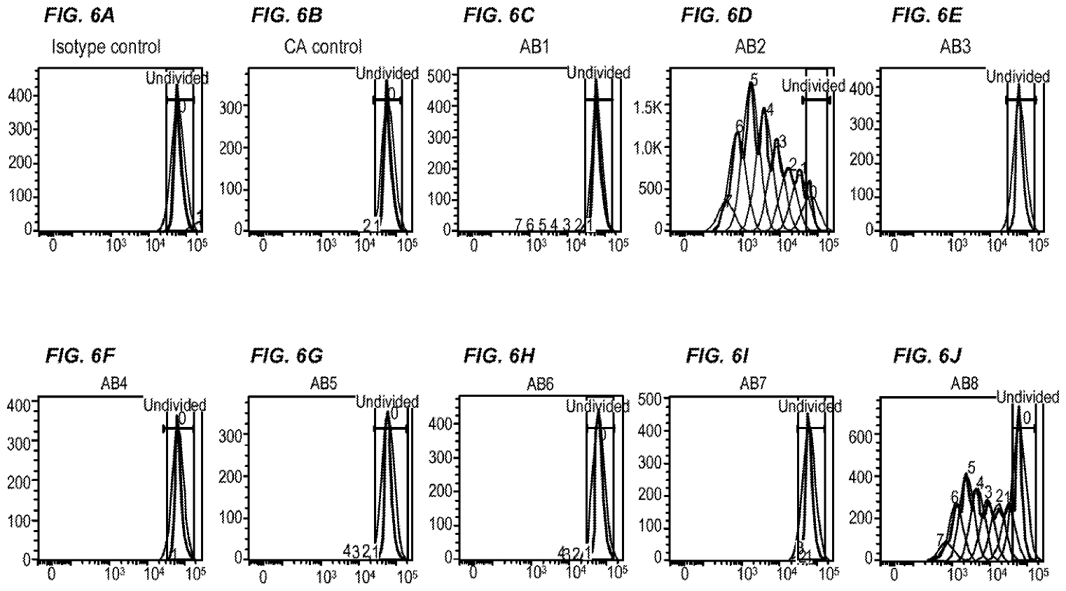

Exemplary anti-CD28 antibodies were assessed for their ability to activate T cells in the absence of a co-stimulatory signal (super-agonistic activity). CFSE labeled T cells were plated on isotype control or exemplary anti-CD28 only coated 12-well plates. After incubation, cells were acquired on a BD Canto II and corresponding data was analyzed by FlowJo. Results are shown in FIGS. 6A-6J, and quantification of results shown in Table E5.

As shown in FIGS. 6A-6J, whereas anti-CD3 alone (FIG. 6A) and the commercially available antibody (FIG. 6B) were not capable of inducing T cell division, exemplary antibodies AB2 and AB8 were capable to enhancing T cell division in the absence of a co-stimulatory anti-CD3 antibody. These results demonstrate the ability of exemplary tested antibodies AB2 and AB8 to enhance CD3-associated T cell activation and proliferation in the absence of co-stimulation.

TABLE E5
Analysis of co-stimulation independent T cell
division using exemplary antibodies

| | Percent | Division | Expansion |
|---|---|---|---|
| | Divided | Index | Index |
| Isotype Control | 3.89 | 0.039 | 1.04 |
| CA control | 0.6 | 0.007 | 1.01 |
| AB1 | 0.58 | 0.0072 | 1.01 |
| AB2 | 65.4 | 1.49 | 6.13 |
| AB3 | 0.32 | 0.0056 | 1.01 |
| AB4 | 0.0091 | 0.0002 | 1 |
| AB5 | 0.64 | 0.0082 | 1.01 |
| AB6 | 0.33 | 0.0047 | 1.01 |
| AB7 | 0.47 | 0.014 | 1.03 |
| AB8 | 30.7 | 0.62 | 2.85 |

### Example 5. Assessment of Binding Specificity of Exemplary Anti-CD28 Antibodies

This Example describes assessment of the ability of exemplary generated anti-CD28 antibodies to compete with CD86 for binding to CD28 on a target cell.

Ba/F3 cells were transfected with human CD28 using standard retroviral transduction protocols, and incubated with or without 70 ug of either purified AB1-AB8 or commercially available control (CA control) or 15 minutes in the dark, after which 1 μg biotinylated recombinant CD86 was added. After 15 minutes of incubation and wash, cells were incubated with PE-conjugated streptavidin. Data were acquired on a BD Canto II, and analyzed using FlowJo software.

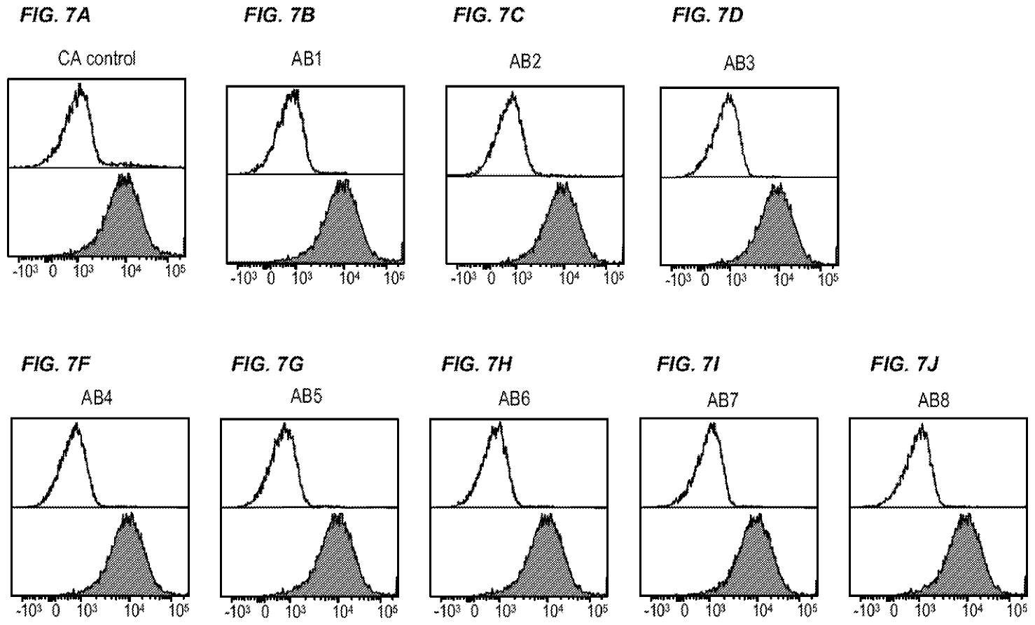

FIGS. 7A-7J depict histogram overlays for cells pre-incubated with exemplary anti-CD28 antibodies AB1-AB8 (top) and without pre-incubation with anti-CD28 antibodies (bottom). Percentage reduction of binding was calculated by dividing the mean fluorescence intensity (MFI) of samples incubated with AB1-AB8 and CA control by the MFI of samples incubated with recombinant CD86 only. This value was subtracted from 100 to get a reduction percentage. The formula is shown below, and results of the calculations are shown in Table E6.

$$\% Reduction = 100 - ( \frac{[ M F I blocking ]}{[ M F I no blocking ]} * 100 )$$

TABLE E6
Competitive binding of exemplary antibodies
for CD28 ligand

| | MFI | % Reduction |
|---|---|---|
| CD86 binding | 8287 | 0 |
| Blocked by CA control | 1066 | 87.1 |
| Blocked by AB1 | 736 | 91.1 |
| Blocked by AB2 | 714 | 91.4 |
| Blocked by AB3 | 796 | 90.4 |
| Blocked by AB4 | 657 | 92.1 |
| Blocked by AB5 | 668 | 91.9 |
| Blocked by AB6 | 741 | 91.1 |
| Blocked by AB7 | 955 | 88.5 |
| Blocked by AB8 | 925 | 88.8 |

As shown in FIGS. 7A-7J and Table E3, exemplary antibodies AB1-AB8 were capable of competing with CD86 for binding to CD28 on target cells.

### Example 6. Assessment of Exemplary Anti-CD28 Antibodies Activity on Non-Anti-CD3 Mediated T Cell Activation Markers

This Example describes assessment of the ability of exemplary generated anti-CD28 antibodies to induce T-cell activation markers without anti-CD3 co-stimulation.

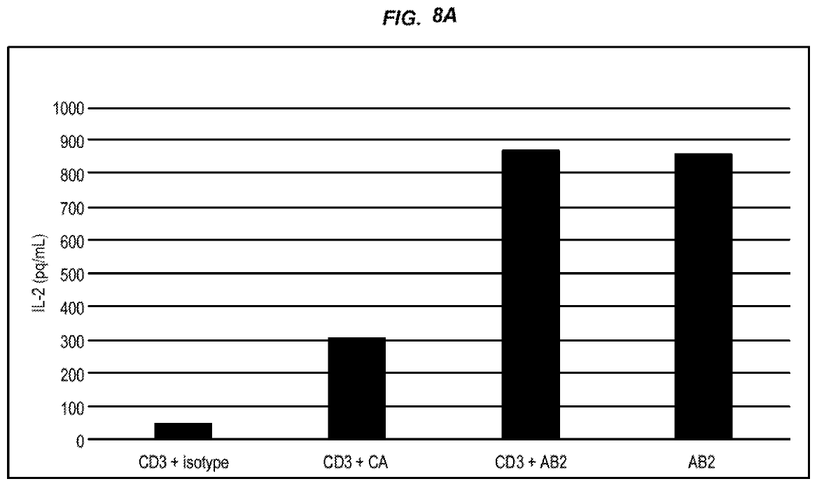

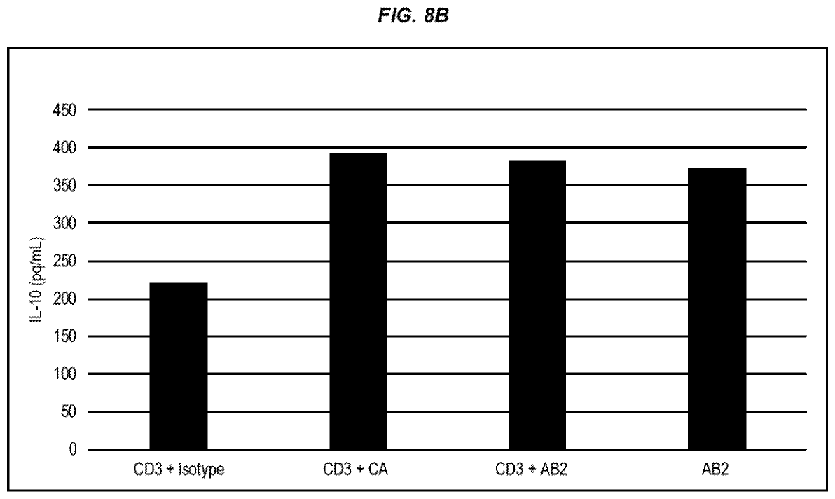

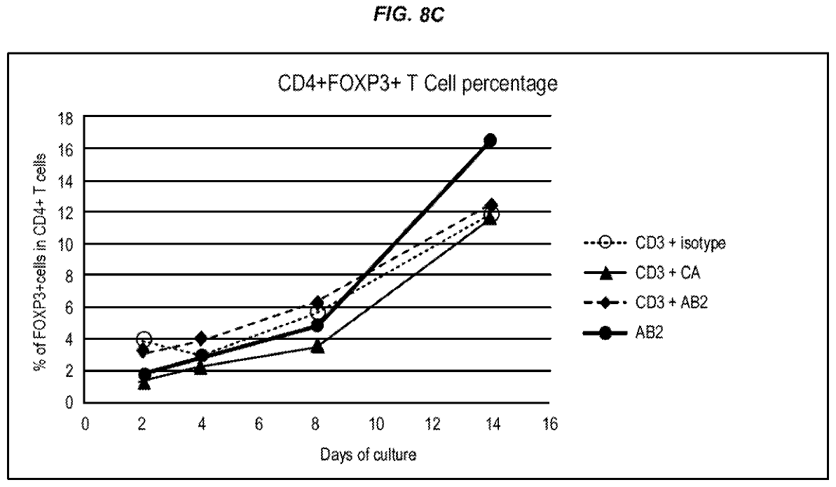

Briefly, total T cells were isolated from freshly collected peripheral blood. Cell were cultured in 6-well plates coated with either an anti-CD3 antibody plus isotype control, a commercially available anti-CD28 antibody (CA), or exemplary antibody AB2, or with exemplary antibody AB2 without anti-CD3. Cell culture supernatants were collected at various time points for analysis as follows: after 2 days of culture, cell culture supernatants were collected; IL-2 concentration was determined by ELISA (FIG. 8A). After 4 days of culture, cell culture supernatants were collected; IL-10 concentration was determined by ELISA (FIG. 8B). At 2, 4, 8, or 14 days of cultures, cells were collected and counted, and flow cytometry analysis was performed to determine percentage of CD4+FOXP3+ T cells (FIG. 8C).

As shown in FIG. 8A and FIG. 8B, exemplary antibody AB2 is capable of inducing T-cell mediated IL-2 and IL-10 production in the absence of anti-CD3 co-stimulation, respectively. In addition, as shown in FIG. 8C, exemplary antibody AB2 is capable of supporting FOXP3+CD4+ T cell growth in the absence of anti-CD3 co-stimulation.

The above examples are provided to illustrate the disclosure but not to limit its scope. Other variants of the disclosure will be readily apparent to one of ordinary skill in the art and are encompassed by the appended claims. All publications, databases, internet sources, patents, patent applications, and accession numbers cited herein are hereby incorporated by reference in their entireties for all purposes.

SEQUENCE TABLE

| SEQ ID | | |
|---|---|---|
| NO | SEQUENCE | DESCRIPTION |
| 1 | QVHLQQSATELARPGASVKMSCKASGYTFTSYTMHWVKQ | Heavy chain |
| | RPGQGLEWIGYIDPGSGYSEFNHKFKDKSTLTADKSSSTAY | amino acid |
| | MQLISLTSEDSAVYYCARRQDNSGYDSFDYWGQGTTLTVS | |
| | S | |
| 2 | QVQLQQPGSELMRPGASVKLSCKASGYTFTSYWMHWVKQ | Heavy chain |
| | RHGQGLEWIGNIYPGSGRTIYDEKFKSKATLTVDTSSSTAY | amino acid |
| | MHLSSLTSEDSAVFYCTRSHYGSSFDAMDYWGQGTSVTVS | |
| | S | |
| 3 | KVQLQQSGAGLVKPGASVKLSCKTSGYTFSDYIINWVKQR | Heavy chain |
| | SGQGLEWIGWFYPGSGSINYNEKFKDKATLTADKSSSTVY | amino acid |
| | MDLSRLTSEDSAVYFCARREDRGGYDAFDYWGQGTTLTV | |
| | SS | |
| 4 | EVQLQQSGPELEKPGASVKMSCKASGYSFTDYNMNWVKQ | Heavy chain |
| | SNGKSLEWIGIIDPYYGATTYNQRFKGKATLTVDKSSSTAY | amino acid |
| | MQLKSLTSEDSAVYYCARFGFYGAMDYWGQGISVTVSS | |
| 5 | KVQLQQSGAGLVKPGASVKLSCKASGYTFSDYIINWVKQR | Heavy chain |
| | SGQGLEWIGWFYPGSGSINYNEKFKDKATLTADKSSSTVY | amino acid |
| | MDLSRLTSEDSAVYFCARREDRGGYDAFDYWGQGTTLTV | |
| | SS | |
| 6 | EVKLVESGGGLVQPGGSLKLSCAASGFTFSSYTMSWVRQT | Heavy chain |
| | PEKRLEWVAYISNGGGTTYYPDTVKGRFTISRDNAKNTLY | amino acid |
| | LQMSSLKSEDTAVYYCARHRPYDYDVLDYWGQGTTLTVS | |
| | S | |
| 7 | DIQMTQSPASLSVSVGETVTITCRTSENIYSNLAWYQQKQG | Light chain |
| | KSPQLLVYAATNLADGVSSRFSGSGSGIQYSLKINSLQSEDF | amino acid |
| | GSYYCQHFWGIPWTFGGGTKLEIK | |
| 8 | DIQVNQSPSSLSASLGDTITITCHASQNIYVWLNWYQQKPG | Light chain |
| | NIPKLLIYKASNLHTGVPSRFSGSGSGTGFTLTISSLQPEDIA | amino acid |
| | TYYCQQGQSYPWTFGGGTKLEIK | |
| 9 | DIQMTQSPASLSVSVGETVTITCRASENIYSNLAWYQQKQG | Light chain |
| | KSPQLLVYAATKLADGVPSRFSGSGSGTQYSLRINSLQSED | amino acid |
| | FGSYYCQHFWGTPWTFGGGTKLEIK | |
| 10 | DVVVTQTPLSLPVSFGDQVSISCRSSQSLANSFGNTYLSWY | Light chain |
| | LHKPGQSPQLLIYGISNRFSGVPDRFSGSGSGTDFTLKISTIK | amino acid |
| | PEDLGVYYCLQGTHQPFTFGSGTTLELK | |
| 11 | DIQMTQSPASLSVSVGETVTITCRTSENIYSNLAWYQQKQG | Light chain |
| | KSPQLLVYAATKLADGVPSRFSGSGSGTQYSLRINSLQPED | amino acid |
| | FGSYYCQHFWGTPWTFGGGTKLEIK | |
| 12 | DIVMTQSQKFMSTSVGDRVSITCKASQIVGTDVAWYQQKP | Light chain |
| | GQSPKILIFSASNRYTGVPDRFTGSGSGTDFTLTISNMQSED | amino acid |
| | LADYFCHQYSRYPYTFGGGTKLEIK | |
| 13 | SYTMH | CDRH1 |
| 14 | SYWMH | CDRH1 |
| 15 | DYIIN | CDRH1 |
| 16 | DYNMN | CDRH1 |
| 17 | SYTMS | CDRH1 |
| 18 | YIDPGSGYSEFNHKFKD | CDRH2 |
| 19 | NIYPGSGRTIYDEKFKS | CDRH2 |
| 20 | WFYPGSGSINYNEKFKD | CDRH2 |
| 21 | IIDPYYGATTYNQRFKG | CDRH2 |
| 22 | YISNGGGTTYYPDTVKG | CDRH2 |
| 23 | RQDNSGYDSFDY | CDRH3 |
| 24 | SHYGSSFDAMDY | CDRH3 |
| 25 | REDRGGYDAFDY | CDRH3 |
| 26 | FGFYGAMDY | CDRH3 |
| 27 | HRPYDYDVLDY | CDRH3 |
| 28 | RTSENIYSNLA | CDRL1 |
| 29 | HASQNIYVWLN | CDRL1 |
| 30 | RASENIYSNLA | CDRL1 |
| 31 | RSSQSLANSFGNTYLS | CDRL1 |
| 32 | KASQIVGTDVA | CDRL1 |
| 33 | AATNLAD | CDRL2 |
| 34 | KASNLHT | CDRL2 |
| 35 | AATKLAD | CDRL2 |
| 36 | GISNRFS | CDRL2 |
| 37 | SASNRYT | CDRL2 |
| 38 | QHFWGIPWT | CDRL3 |
| 39 | QQGQSYPWT | CDRL3 |
| 40 | QHFWGTPWT | CDRL3 |
| 41 | LQGTHQPFT | CDRL3 |
| 42 | HQYSRYPYT | CDRL3 |
| 43 | CAGGTCCACCTGCAGCAGTCTGCAACTGAACTGGCAAGA | Heavy chain |
| | CCTGGGGCCTCAGTGAAGATGTCCTGCAAGGCTTCTGGC | nucleic acid |
| | TACACCTTTACTAGCTACACGATGCACTGGGTAAAACAG | |
| | AGGCCTGGACAGGGTCTGGAATGGATTGGATACATTGAT | |
| | CCTGGCAGTGGATATTCTGAGTTCAATCACAAGTTCAAG | |
| | GACAAGAGCACATTGACTGCAGACAAATCCTCCAGCAC | |
| | AGCCTACATGCAACTGATCAGCCTGACATCTGAGGACTC | |
| | TGCGGTCTATTACTGTGCAAGAAGGCAGGACAACTCGGG | |
| | CTACGACTCCTTTGACTACTGGGGCCAAGGCACTACTCT | |
| | CACAGTCTCCTCA | |
| 44 | CAGGTCCAACTGCAGCAACCTGGGTCTGAGCTGATGAGG | Heavy chain |
| | CCTGGAGCTTCAGTGAAGCTGTCCTGCAAGGCTTCTGGC | nucleic acid |
| | TACACATTCACCAGCTACTGGATGCACTGGGTGAAGCAG | |
| | AGGCATGGACAAGGCCTTGAGTGGATTGGAAATATTTAT | |
| | CCTGGTAGTGGTAGAACTATCTACGATGAGAAGTTCAAG | |
| | AGCAAGGCCACACTGACTGTAGACACATCCTCCAGCACA | |
| | GCCTACATGCACCTCAGCAGCCTGACATCTGAGGACTCT | |
| | GCGGTCTTTTACTGTACAAGATCCCACTACGGTAGTAGC | |
| | TTCGATGCTATGGACTACTGGGGTCAAGGAACCTCAGTC | |
| | ACCGTCTCCTCA | |
| 45 | AAGGTCCAGCTGCAGCAGTCTGGAGCTGGGCTGGTGAA | Heavy chain |
| | ACCCGGGGCATCAGTGAAGCTGTCCTGCAAGACTTCTGG | nucleic acid |
| | CTACACCTTCAGTGACTATATTATAAATTGGGTAAAGCA | |
| | GAGGTCTGGACAGGGTCTTGAGTGGATTGGGTGGTTTTA | |
| | CCCTGGAAGTGGTAGTATAAATTACAATGAGAAATTCAA | |
| | GGACAAGGCCACATTGACTGCGGACAAATCCTCCAGCA | |
| | CGGTCTATATGGACCTTAGTAGATTGACGTCTGAAGACT | |
| | CTGCGGTCTATTTCTGTGCAAGACGCGAAGATCGGGGAG | |
| | GTTACGACGCCTTTGACTACTGGGGCCAAGGCACCACTC | |
| | TCACAGTCTCCTCA | |
| 46 | GAGGTCCAGCTGCAGCAGTCTGGACCTGAGCTGGAGAA | Heavy chain |
| | GCCTGGCGCTTCAGTGAAGATGTCCTGCAAGGCTTCTGG | nucleic acid |
| | TTACTCATTCACTGACTACAACATGAACTGGGTGAAGCA | |
| | GAGCAATGGAAAGAGCCTTGAGTGGATTGGAATTATTG | |
| | ATCCTTACTATGGTGCTACTACCTACAACCAGAGGTTCA | |
| | AGGGCAAGGCCACATTGACTGTAGACAAATCCTCCAGC | |
| | ACAGCCTACATGCAGCTCAAGAGCCTGACATCTGAGGAC | |
| | TCTGCAGTCTATTACTGTGCAAGATTTGGTTTCTACGGGG | |
| | CTATGGACTACTGGGGTCAAGGAATTTCAGTCACCGTCT | |
| | CCTCA | |
| 47 | GAGGTCCAGCTGCAGCAGTCTGGACCTGAGCTGGAGAA | Heavy chain |
| | GCCTGGCGCTTCAGTGAAGATGTCCTGCAAGGCTTCTGG | nucleic acid |
| | TTACTCATTCACTGACTACAACATGAACTGGGTGAAGCA | |
| | GAGCAATGGAAAGAGCCTTGAGTGGATTGGAATTATTG | |
| | ATCCTTACTATGGTGCTACTACCTACAACCAGAGGTTCA | |
| | AGGGCAAGGCCACATTGACTGTAGACAAATCCTCCAGC | |
| | ACAGCCTACATGCAGCTCAAGAGCCTGACATCTGAGGAC | |
| | TCTGCAGTCTATTACTGTGCAAGATTTGGTTTCTACGGGG | |
| | CTATGGACTACTGGGGTCAAGGAATTTCAGTCACCGTCT | |
| | CCTCA | |
| 48 | AAGGTCCAGCTGCAGCAGTCTGGAGCTGGGCTGGTGAA | Heavy chain |
| | ACCCGGGGCATCAGTGAAGCTGTCCTGCAAGACTTCTGG | nucleic acid |
| | CTACACCTTCAGTGACTATATTATAAATTGGGTAAAGCA | |
| | GAGGTCTGGACAGGGTCTTGAGTGGATTGGGTGGTTTTA | |
| | CCCTGGAAGTGGTAGTATAAATTACAATGAGAAATTCAA | |
| | GGACAAGGCCACATTGACTGCGGACAAATCCTCCAGCA | |
| | CGGTCTATATGGACCTTAGTAGATTGACGTCTGAAGACT | |
| | CTGCGGTCTATTTCTGTGCAAGACGCGAAGATCGGGGAG | |
| | GTTACGACGCCTTTGACTACTGGGGCCAAGGCACCACTC | |
| | TCACAGTCTCCTCA | |
| 49 | AAGGTCCAGCTGCAGCAGTCTGGAGCTGGGCTGGTGAA | Heavy chain |
| | ACCCGGGGCATCAGTGAAGCTGTCCTGCAAGGCTTCTGG | nucleic acid |
| | CTACACCTTCAGTGACTATATTATAAATTGGGTAAAGCA | |
| | GAGGTCTGGACAGGGTCTTGAGTGGATTGGGTGGTTTTA | |
| | CCCTGGAAGTGGTAGTATAAATTACAATGAGAAATTCAA | |
| | GGACAAGGCCACATTGACTGCGGACAAATCCTCCAGCA | |
| | CAGTCTATATGGACCTTAGTAGATTGACATCTGAAGACT | |
| | CTGCGGTCTATTTCTGTGCAAGACGCGAAGATCGGGGAG | |
| | GTTACGACGCCTTTGACTACTGGGGCCAAGGCACCACTC | |
| | TCACAGTCTCCTCA | |
| 50 | GAAGTGAAGCTGGTGGAGTCTGGGGGAGGTTTAGTGCA | Heavy chain |
| | GCCTGGAGGGTCCCTGAAACTCTCCTGTGCAGCCTCTGG | nucleic acid |
| | ATTCACTTTCAGTAGCTATACCATGTCTTGGGTTCGCCAG | |
| | ACTCCAGAGAAGAGGCTGGAGTGGGTCGCATACATTAG | |
| | TAATGGTGGTGGTACTACCTACTATCCAGACACTGTAAA | |
| | GGGCCGATTCACCATCTCCAGAGACAATGCCAAGAACA | |
| | CCCTGTACCTGCAAATGAGCAGTCTGAAGTCTGAGGACA | |
| | CGGCCGTGTATTACTGTGCAAGGCATAGGCCCTATGATT | |
| | ACGACGTCCTTGACTACTGGGGCCAAGGCACCACTCTCA | |
| | CAGTCTCCTCA | |
| 51 | GACATCCAGATGACTCAGTCTCCAGCCTCCCTATCTGTA | Light chain |
| | TCTGTGGGAGAAACTGTCACCATCACATGTCGAACAAGT | nucleic acid |
| | GAGAATATTTACAGTAATTTAGCATGGTATCAGCAGAAA | |
| | CAGGGAAAATCTCCTCAGCTCCTGGTCTATGCTGCAACA | |
| | AACTTAGCAGATGGTGTGTCATCAAGGTTCAGTGGCAGT | |
| | GGATCAGGCATACAGTATTCCCTCAAGATCAACAGCCTG | |
| | CAGTCTGAAGATTTTGGGAGTTATTACTGTCAACATTTTT | |
| | GGGGTATTCCGTGGACGTTCGGAGGAGGCACCAAGCTG | |
| | GAAATCAAA | |
| 52 | GACATCCAGGTGAACCAGTCTCCATCCAGTCTGTCTGCA | Light chain |
| | TCCCTTGGAGACACAATTACCATCACTTGCCATGCCAGT | nucleic acid |
| | CAGAACATTTATGTTTGGTTAAACTGGTACCAGCAGAAA | |
| | CCAGGAAATATTCCTAAACTATTGATCTATAAGGCTTCC | |
| | AACTTGCACACAGGCGTCCCATCAAGGTTTAGTGGCAGT | |
| | GGATCTGGGACAGGTTTCACATTAACCATCAGCAGCCTG | |
| | CAGCCTGAAGACATTGCCACTTACTACTGTCAACAGGGT | |
| | CAAAGTTATCCGTGGACGTTCGGTGGAGGCACCAAGCTG | |
| | GAAATCAAA | |
| 53 | GACATCCAGATGACTCAGTCTCCAGCCTCCCTATCTGTTT | Light chain |
| | CTGTGGGAGAAACTGTCACCATCACATGTCGAGCAAGTG | nucleic acid |
| | AGAATATTTACAGTAATTTAGCATGGTATCAGCAGAAAC | |
| | AGGGAAAATCTCCTCAGCTCCTGGTCTATGCTGCAACAA | |
| | AATTAGCAGATGGTGTGCCATCAAGGTTCAGTGGCAGTG | |
| | GATCAGGCACACAGTATTCCCTCAGGATCAACAGCCTGC | |
| | AGTCTGAAGATTTTGGGAGTTATTACTGTCAACATTTTTG | |
| | GGGTACTCCGTGGACGTTCGGTGGAGGCACCAAGCTGG | |
| | AAATCAAA | |
| 54 | GACATCCAGATGACTCAGTCTCCAGCCTCCCTATCTGTTT | Light chain |
| | CTGTGGGAGAAACTGTCACCATCACATGTCGAGCAAGTG | nucleic acid 2 |
| | AGAATATTTACAGTAATTTAGCATGGTATCAGCAGAAAC | |
| | AGGGAAAATCTCCTCAGCTCCTGGTCTATGCTGCAACAA | |
| | AATTAGCAGATGGTGTGCCATCAAGGTTCAGTGGCAGTG | |
| | GATCAGGCACACAGTATTCCCTCAGGATCAACAGCCTGC | |
| | AGTCTGAAGATTTTGGGAGTTATTACTGTCAACATTTTTG | |
| | GGGTACTCCGTGGACGTTCGGTGGAGGCACCAAGCTGG | |
| | AAATCAAA | |
| 55 | GATGTTGTGGTGACTCAAACTCCACTCTCCCTGCCTGTCA | Light chain |
| | GCTTTGGAGATCAAGTTTCTATCTCTTGCAGGTCTAGTCA | nucleic acid |
| | GAGTCTTGCAAACAGTTTTGGGAACACCTATTTGTCTTG | |
| | GTACCTGCACAAGCCTGGCCAGTCTCCACAGCTCCTCAT | |
| | CTATGGGATTTCCAACAGATTTTCTGGGGTGCCAGACAG | |
| | GTTCAGTGGCAGTGGTTCAGGGACAGATTTCACACTCAA | |
| | GATCAGCACAATAAAGCCTGAGGACTTGGGAGTGTATTA | |
| | CTGCTTACAAGGTACACATCAACCATTCACGTTCGGCTC | |
| | GGGGACAACGTTGGAACTAAAA | |
| 56 | GACATCCAGATGACTCAGTCTCCAGCCTCCCTATCTGTTT | Light chain |
| | CTGTGGGAGAAACTGTCACCATCACATGTCGAGCAAGTG | nucleic acid |
| | AGAATATTTACAGTAATTTAGCATGGTATCAGCAGAAAC | |
| | AGGGAAAATCTCCTCAGCTCCTGGTCTATGCTGCAACAA | |
| | AATTAGCAGATGGTGTGCCATCAAGGTTCAGTGGCAGTG | |
| | GATCAGGCACACAGTATTCCCTCAGGATCAACAGCCTGC | |
| | AGTCTGAAGATTTTGGGAGTTATTACTGTCAACATTTTTG | |
| | GGGTACTCCGTGGACGTTCGGTGGAGGCACCAAGCTGG | |
| | AAATCAAA | |
| 57 | GACATCCAGATGACTCAGTCTCCAGCCTCCCTATCTGTA | Light chain |
| | TCTGTGGGAGAAACTGTCACCATCACATGTCGAACAAGT | nucleic acid |
| | GAGAATATTTACAGTAATTTAGCATGGTATCAGCAGAAA | |
| | CAGGGAAAATCTCCTCAGCTCCTGGTCTATGCTGCAACA | |
| | AAGTTAGCAGATGGTGTGCCATCAAGGTTCAGTGGCAGT | |
| | GGATCAGGCACACAGTATTCCCTCAGGATCAACAGCCTG | |
| | CAGCCTGAAGATTTTGGGAGTTATTACTGTCAACATTTTT | |
| | GGGGTACTCCGTGGACGTTCGGTGGAGGCACCAAGCTG | |
| | GAAATCAAA | |
| 58 | GACATTGTGATGACCCAGTCTCAAAAATTCATGTCCACA | Light chain |
| | TCAGTCGGAGACAGGGTCAGCATCACCTGTAAGGCCAGT | nucleic acid |
| | CAGATTGTGGGTACTGATGTAGCCTGGTATCAACAGAAA | |
| | CCAGGACAGTCTCCTAAAATATTGATTTTTTCGGCATCC | |
| | AATCGGTACACTGGAGTCCCTGATCGCTTCACAGGCAGT | |
| | GGATCTGGGACAGATTTCACTCTCACCATCAGCAATATG | |
| | CAGTCTGAAGACCTGGCAGATTATTTCTGCCATCAATAT | |
| | AGCAGGTATCCGTACACGTTCGGAGGGGGGACCAAGCT | |
| | GGAAATAAAA | |
| 59 | ATGGAAAGGCACTGGATCTTTCTCTTCCTGTTGTCAGTA | Heavy chain |
| | ACTGCAGGTGTCCACTCC | signal sequence |
| 60 | ATGGGATGGAGCTGTATCATCCTCTTCTTGGTAGCAACA | Heavy chain |
| | GCTACAGGTGTCCACTCC | signal sequence |
| 61 | ATGGAATGGTGCTGGGTCTTTCTCTTCCTCCTGTCAGTAA | Heavy chain |
| | CTGCAGGTGTCCACTCC | signal sequence |
| 62 | ATGGGATGGACCTGGATCTTTATTTTAATCCTGTCAGTA | Heavy chain |
| | ACTACAGGTGTCCACTCT | signal sequence |
| 63 | ATGAATTTCGGGCTCAGCTTGATTTTCCTTGTCCTTGTTT | Heavy chain |
| | TAAAAGGTGTCCTGTGT | signal sequence |
| 64 | ATGAGTGTGCCCACTCAGGTCCTGGGGTTGCTGCTGCTG | Light chain signal |
| | TGGCTTACAGATGCCAGATGT | sequence |
| 65 | ATGAGGGTCCTTGCTGAGCTCCTGGGGCTGCTGCTGTTC | Light chain signal |
| | TGCTTTTTAGGTGTGAGATGT | sequence |
| 66 | ATGAATTTGCCTGTTCATCTCTTGGTGCTTCTGTTGTTCT | Light chain signal |
| | GGATTCCCGCTTCCAGAGGT | sequence |
| 67 | ATGGAGTCTCATACTCAGGCCTTTGTATTCGCGTTTCTCT | Light chain signal |
| | GGTTGTCTGGTGTTGATGGA | sequence |
| 68 | X1YX3X4X5 | CDRH1 |
| | | X1 is S or D; |
| | | X3 is T, W, I or |
| | | N; |
| | | X4 Is M or I; |
| | | X5 is H, N or S |
| 69 | X1X2X3X4X5X6GX8X9X10X11X12X13X14X15KX17 | CDRH2 |
| | | X1 is Y, N, W or I; |
| | | X2 is I or F; X3 is |
| | | D, Y or S; X4 is |
| | | P or N, X5 is G |
| | | or Y; X6 is S, Y |
| | | or G; X8 is Y, R, |
| | | S, A or T; X9 is |
| | | S, T or I; X10 is |
| | | E, I, N, T or Y; |
| | | X11 is F or Y; |
| | | X12 is N, D or P; |
| | | X13 is H, E, Q or |
| | | D; X14 is K, R or |
| | | T; X15 is F or V; |
| | | X17 is D, S or G |
| 70 | X1X2X3X4X5X6X7X8X9X10DY | CDRH3 |
| | | X1 is R, S, F or H; |
| | | X2 is Q, H, E, G |
| | | or R; X3 is D, Y |
| | | F or P; X4 is N, |
| | | G, R, Y or T; X5 |
| | | is S, G or D; X6 |
| | | is G, S or no |
| | | amino acid; X7 is |
| | | Y, F or no amino |
| | | acid; X8 is D or |
| | | no amino acid; |
| | | X9 is S, A or V; |
| | | X10 is F, M or L |
| 71 | X1X2SX4X5X6X7X8X9X10X11X12X13X14X15X16 | CDRL1 |
| | | X1 is R, H or K; |
| | | X2 is T, A or S; |
| | | X4 is E or Q; X5 |
| | | is N, S or I; X6 is |
| | | I, L or V; X7 is |
| | | Y, A or G; X8 is |
| | | S, V, N or T; X9 |
| | | is N, W, S or D; |
| | | X10 is L, F or V; |
| | | X11 is A, N or G; |
| | | X12 is N or no |
| | | amino acid; X13 |
| | | is T or no amino |
| | | acid; X14 is Y or |
| | | no amino acid; |
| | | X15 is L or no |
| | | amino acid; X16 |
| | | is S or no amino |
| | | acid |
| 72 | X1X2X3X4X5X6X7 | CDRL2 |
| | | X1 is A, K, G or |
| | | S; X2 is A or I; |
| | | X3 is T or S; X4 |
| | | is N or K; X5 is L |
| | | or R; X6 is A, H, |
| | | F or Y; X7 is D, |
| | | T or S |
| 73 | X1X2X3X4X5X6PX8T | CDRL3 |
| | | X1 is Q, L or H; |
| | | X2 is H or Q; X3 |
| | | is F, G or Y; X4 |
| | | is W, Q, T or S; |
| | | X5 is G, S H or |
| | | R; X6 is I, Y, T |
| | | or Q; X8 is W, F |
| | | or Y |
| 74 | NKILVKQSPMLVAYDNAVNLSCKYSYNLFSREFRASLHKG | CD28 isoform 1 |
| | LDSAVEVCVVYGNYSQQLQVYSKTGFNCDGKLGNESVTF | |
| | YLQNLYVNQTDIYFCKIEVMYPP | |
| 75 | KHLCPSPLFPGPSKPFWVLVVVGGVLACYSLLVTVAFIIFW | CD28 isoform 2 |
| | V | |
| 76 | RSKRSRLLHSDYMNMTPRRPGPTRKHYQPYAPPRDFAA | CD28 isoform 3 |
| 77 | NKILVKQSPMLVAYDNAVNLSWKHLCPSPLFPGPSKPFWV | CD28 isoform 4 |
| | LVVVGGVLACYSLLVTVAFIIFWVRSKRSRLLHSDYMNMT | |
| | PRRPGPTRKHYQPYAPPRDFAAYRS | |
| 78 | NKILVKQSPMLVAYDNAVNLSYNEKSNGTIIHVKGEE | CD28 isoform 5 |
| 79 | NKILVKQSPMLVAYDNAVNLSYNEKSNGTIIHVKGKHLCP | CD28 isoform 6 |
| | SPLFPGPSKPYAPPRDFAAYRS | |
| 80 | NKILVKQSPMLVAYDNAVNLSCKYSYNL | CD28 isoform 7 |
| | FSREFRASLHKGLDSAVEVCVVYGNYSQQLQVYSKTGFNC | |
| | DGKLGNESVTFYLQNLYVNQ | |
| | TDIYFCKIEVMYPPPYLDNEKSNGTIIHVKGKHLCPSPLFPG | |
| | PSKPFWVLVVVGGVLACY | |
| | SLLVTVAFIIFWVRSKRSRLLHSDYMNMTPRRPGPTRKHYQ | |
| | PYAPPRDFAAYRS | |
| 81 | NKILVKQSPMLVAYDNAVNLSCKYSYNLFSREFRASLHKG | CD28 |
| | LDSAVEVCVVYGNYSQQLQV | extracellular |
| | YSKTGFNCDGKLGNESVTFYLQNLYVNQTDIYFCKIEVMY | |
| | PPPYLDNEKSNGTIIHVKGK | |
| | HLCPSPLFPGPSKP | |
| 82 | FWVLVVVGGVLACYSLLVTVAFIIFWV | CD28 |
| | | transmembrane |
| 83 | RSKRSRLLHSDYMNMTPRRPGPTRKHYQPYAPPRDFAAYR | CD28 |
| | S | cytoplasmic |
| 84 | MLRLLLALNLFPSIQVTG | CD28 signal |
| | | peptide |
| 85 | APELLGGPSVFLFPPKPKDTLMISRTPEVTCVVVDVSHEDPE | Fc |
| | VKFNWYVDGVEVHNAKTKPREEQYNSTYRVVSVLTVLHQ | |
| | DWLNGKEYKCKVSNKALPAPIEKTISKAKGQPREPQVYTL | |
| | PPSRDELTKNQVSLTCLVKGFYPSDIAVEWESNGQPENNYK | |
| | TTPPVLDSDGSFFLYSKLTVDKSRWQQGNVFSCSVMHEAL | |
| | HNHYTQKSLSLSPGK | |

1-31. (canceled)

32. An isolated antibody or antigen-binding fragment thereof that specifically binds to CD28, wherein the isolated antibody or antigen-binding fragment thereof comprises: (i) an immunoglobulin heavy chain variable domain comprising heavy chain complementarity determining region (CDR) amino acid sequences, CDRH1, CDRH2, and CDRH3; and, (ii) an immunoglobulin light chain variable domain comprising light chain CDR amino acid sequences, CDRL1, CDRL2, and CDRL3, wherein the heavy chain CDRs and light chain CDRs are each associated with one of antibody AB2, AB3 or AB8 as set forth herein:

| Antibody | CDRH1 | CDRH2 | CDRH3) |
|---|---|---|---|
| AB2 | SEQ ID NO: 14 | SEQ ID NO: 19 | SEQ ID NO: 24 |
| AB3 | SEQ ID NO: 15 | SEQ ID NO: 20 | SEQ ID NO: 25 |
| AB8 | SEQ ID NO: 17 | SEQ ID NO: 22 | SEQ ID NO: 27 |
| Antibody | CDRL1 | CDRL2 | CDRL3 |
| AB2 | SEQ ID NO: 29 | SEQ ID NO: 34 | SEQ ID NO: 39 |
| AB3 | SEQ ID NO: 30 | SEQ ID NO: 35 | SEQ ID NO: 40 |
| AB8 | SEQ ID NO: 32 | SEQ ID NO: 37 | SEQ ID NO: 42 |

33. An isolated nucleic acid encoding the isolated antibody or antigen-binding fragment thereof of claim 32.

34. An expression vector comprising the nucleic acid of claim 33.

35. An isolated host cell comprising the expression vector of claim 34.

36. The isolated antibody or antigen-binding fragment thereof of claim 32, wherein the heavy chain variable domain comprises a polypeptide that is identical to the amino acid sequence set forth in SEQ ID NO: 2, 3, or 6, or at least 80% identical to the amino acid sequence set forth in SEQ ID NO: 2, 3, or 6.

37. An isolated nucleic acid encoding the isolated antibody or antigen-binding fragment thereof of claim 36.

38. An expression vector comprising the nucleic acid of claim 37.

39. An isolated host cell comprising the expression vector of claim 38.

40. The isolated antibody or antigen-binding fragment thereof of claim 32, wherein the light chain variable domain comprises a polypeptide that is identical to the amino acid sequence set forth in SEQ ID NO: 8, 9, or 12, or at least 80% identical to the amino acid sequence set forth in SEQ ID NO:8, 9, or 12.

41. An isolated nucleic acid encoding the isolated antibody or antigen-binding fragment thereof of claim 40.

42. An expression vector comprising the nucleic acid of claim 41.

43. An isolated host cell comprising the expression vector of claim 42.

44. The isolated antibody or antigen-binding fragment thereof of claim 32, further comprising an Fc polypeptide comprising SEQ ID NO:85 or a sequence having at least 90% identity to the sequence of SEQ ID NO:85.

45. A pharmaceutical composition comprising the isolated antibody or antigen-binding fragment thereof of claim 32 and a pharmaceutically acceptable carrier.

46. A kit comprising the isolated antibody or antigen-binding fragment thereof of claim 32.

47. A method of detecting the presence or absence of CD28 in an in vitro sample, the method comprising:
a) contacting the in vitro sample with the isolated antibody or antigen-binding fragment thereof of claim 32, under conditions to bind said antibody or antigen-binding fragment thereof to CD28 in said sample, wherein the binding generates the production of at least one complex comprising CD28 and the antibody or antigen-binding fragment thereof, and
b) detecting the presence or absence of the produced at least one complex comprising CD28 and the antibody or antigen-binding fragment thereof to detect the presence or absence of CD28 in the in vitro sample.

48. An isolated nucleic acid comprising a nucleotide sequence that encodes an immunoglobulin heavy chain variable domain comprising the sequence set forth in SEQ ID NO: 44, 45, or 50.

49. The isolated nucleic acid of claim 48, wherein the nucleic acid further comprises a nucleotide sequence that encodes the immunoglobulin light chain variable domain comprising the sequence set forth in SEQ ID NO: 52, 53, or 58.

50. An expression vector comprising the nucleic acid of claim 49.

51. An isolated host cell comprising the expression vector of claim 50.
